SEQUENCE LISTING (3 sequences)

SEQ ID NO: 1 - DNA, 12959 bp, Homo sapiens
acgcattcatcacatcagtgtttataataaccaggagtaggaggcaatcaaatgtccttcacaagtggaactggtaagttaaagagacttaggttgggtttctctaaaaccaggccacaagacaaagatttgtattccagtggcttattttgatttaggagatgatttaaggaatcaccagtgcgggagtataacagtgaacccaagacaccttgagatcaataaaaaggtgcattgttggcaggctgcctgcaaagaaagggagcataatcccagtagacaacaccaggaggcagttttgtacatgcctaagagtaatcccaccttagcatgtagaacacaaggatatttagtctccagttcccatcatgtgggctgagtggtggtcccaggtgctttaatttgtagtccatctgcccaagcacaggccaaaagaaagccttcccacagagtcccgagttcatgtggcagcatgccagaggtatgtactggaacagtaggtgctaaaggcaacaattacatcatgaaaactgcacatctcactcatatgtggaatctaaaagtaatttagctcatagaagttaagagtagaatagttagagtagtagaaagttaagagttgttactggagagaaaagagggtatggaagctagaaaaaaagctagaaggaaggattttgaatgttctcatgacaaagagatgataaatgtttgaggtgacagacatgctaattaccatgatttgatctttacacaatgtctgcatgtatcagaacatcacacactaccaaataaatatatacaattgttatgtgtcaaaaaacattatatacaatgtttatattttatattaatattaatgtaaataaaaatttacacaaatcttttcattaaatatggtgtggataattatattatctgatgatacccatgccacaggctaggagaaataaaaatttacattaatgttaatgtcaaaaaagcagaaacaaagccatatgatacatgtgacacttataccacatgcatagagttatttcagacacattgtgtgttacctttgggtagagaagtgaatgagagtgaggatgagagatgaaaagaggcagaagaaaaagagaggccttataggaagtgatgaaagtgtgccataaactgagagtatgactaaaacctttcatcttcctcttctgccgcttctaagttattggttctgctgttccttgacctgtttcaagctctcaccttctatatcacacatgggaagtctggcatcagacttccagagagcaagaactaggtgaaatacaagggcacagctctcctagcctgtggcatgggtatcatcagataatataattatccacaccatttttaacaaaaagatttgcgtaagagattcacagaggcaacctgaggccctgcaactacatttcccagagatccctgaggtgatcctaactagactctggtgtcagggtgatacggaattccagtgagatcacttcccttgcagactttggaagggagagcactttattacagaccttggaagcaagaggattgcattcagcctagttcctggttgctggccaaagtaagtagaactttgtaagtaatttgcagtgtactttgaaagtggggtataaattaaaaataaaatcaagtccccctactgactgaatggatcccctcttgtcctaggcgaccccagagaaacctggaaaactaaattccaggccataatggaaagggaggtcagacacgcctcattatacacactcccttttggagttcaggcacaactgaccagcattaacattgaaacagtgatcataagactgtcaaaatggactgtttgtgggaataagataccaaattccaatctgactctggtttagcatcacatgacaatagcagaccctgaaggaaatcaatatatttaatccccaaaatatatttctttgacacatattgaaatggtcttgcaaaaccatcttttatggggaaatttgctggtttctggctctgctctgattgaggagagaataaatgagagtctgacaccttaaaaatctgaaaagagatatttaccatctgttttctctaaggctgctagctggaggtttcatctacatgacataaaccatggttcccataagcccttttaactcaagcatttaattgtactgaccgcaagtctttagacaaaacttaactcttcaacaaattgccaatcacaaaatctttgaatccacctttgtcctgtaagctctcctacttcaatatgtctgattttcaggcagagccaatgtataccttatgtgggggccataggctgttggccccctaaagcttcactaaaagtcactgtcatgtgacaggttgattaataggggaaagggagtacaaatttatttaatgtatatacatggtctattagccaggaccctttagagggccagaactaataggatagatgtatacataaaggggagtttattaaggagtagtgagtcacatgatcacaaggtgacgtcccacagtaggccgtctgcaagctgaggagcaaggaagccagtccgagtcccaaaacctcaaaagtagggaagccgacagtgcagccttcagtctgtggtcaaaggtccaggagtccaaaggctgaagaacttggagtttgatgtttgagggcaggaagcatccagcacaggagaaagatgtaggctgggagactaagccagtctagtctttccacattcttctgcctactttcattctggctgtgctggcagctgattagatggtgcccacccggattgagggtgggcctccctttctcagtccactgattcaaatgttaatttcctttggcaacaccctcacagacacacccaggaacaatactttgaatccttcaatccaatcaagttgacactcaatattaaccaccacacttgggaaacttgagaatgaagacccaacttcccagtaagttacagaaactcatgtattattccgaggccacagtaaagagtggagactcaaagcatggccagaaacaggtaaattagttttagtggcaacacaggttaagagagagaggaaggaagaagctcggctagcaaaggtggccttgttatgtagacgaaccttttctcagaagcaaaagatggtaaatgtttctatccagatctttaaaggtgtcagactctcaatcgctctggatcctgggaaaggcttagaaacaggagagggtatggctgcattaatggacattgtccacagacgcaaattttcctcagttaagacagttttgcaatgccacttctgcttgttggccagtggaaaccatttttaaatatgtcaaagaaatatatttgagggtaaaatattttgattttcttcagtccccactttgaaatttaaaacaagtttcacatactaaaagccaaggtaatagctttggggagatttaagttagaggtgtttagatagagacagtcatagcagtggaaaaacacatttgggataaatataaatgagcaaatttaaatatatcattccatattttctttaggcagtcccttggttttgaggtgagatcagttcagttaaatatctacgtcccattccaggaggtgggactgcaaatggtttaggcctttagacgtaatgtaggcaatcagatttctagtaggaggcatttctgtagaaacagaaaaaaataaataaataaatgttactatctggagtagtctatatatctagaaaagctagtctatagctttttctctgaggcgtcttcatattgcagtggcaatgtcccagatttccctggaagttcaaaccaggtgttccactgaactttctgagaaggcctcacatcagcagatatgcatgaaggttgtttataagttactgttattttctcctgataccttcagtttgcaagactttaagaaaaccccagttttaatttctagtgaattcaagtaagaaaaatgggagaaaacgttgaaaatgttactttgcagacttgtagccagaaaaaaattcaggatttggtctaacttgtaaaaaataataaaaactgaaaaaacaatgggcaaggctagaagatactaacagatacactatagtttttgaaacatagtatttctgcttccagttcttcaattttatgaaagacatagtttcatgcacatccgtgtgaagagaccaccaaacagactctgtgtgagcaacatggctgtttatttcacctgggtgcaggcgggctgagtcctaaaagagagtcagtgaagggagataagggtggggccattttataggatttggatagataaaagaaaagtacagtcaaaggggggttgttctctggcaggcagagtgggggtcacaaggtgctcagtaggggagctattgagccaggatgagccaggagaaggaatttcacaagacagtgtcatcagttaaggcaggaacaggccattttcccatcttttgtggtggaatgtcatcagtaaggcaggaaccggccatctggatgtgtacatgcaggtcacgggatatgatggcttagcttaggctcagaggcctgacacacagtagaactgatttatttgcaaaataagttttgttactatacttggtctgattttttgcataaagggcagcaagaataattatttactatataggctcctttttaaaaaatggctttgctggaacattcttttttgtaaagaatctcatattagacctcttaaaagcatcctgagcccaaccaaggatttatctgtttctacgagatacctgtatgaattaagtgaattctctctcaaggtcccaaaataagttggggttcctgggcctgtcagaatgtgaccttcttttacccattacaggccagaaacctgtaaaggaacctgtaaaggaactgcatagacaagctgtgaggccagttttatccacagggctattatcagctttataaagtcccaatatcaatttcttaaaaatagggcattccagtcaaatccttgatttttgtctccacttgtggcctgttacaaaacaaaacagattctaactgaatttatgcaaataattatattcccataaattagcaataatcactaagagtttccaaatttcagagaaatcaggcagatagagggaaatatgcttcaaattttgctcacaagagtatactttacacaaatattaaaagctgtaaatgtctaaaaagaaaaaaattttgactctgaaaaacaaaagtggcaatgtttcaacaaaacccataaaaagattatttcagtcttctattatttcagtccatgcaattaactcctattctgttagatattgggtgagcaatcttcatgaacatattagttctccatgagagtcctgggagttttttttctctataccagtggcacaatctccaaagttatcataaagcttcattcaaaagcaccccttagagtcctatagctaattgcaaaccacctttttaaaagcaccaaagtagaacaactgtcaatgacagaagtcttaggacagccatggtcaaggacacacttaacaaggaaatttggttatttctgtggcatataacaatttaacataataattataaatactaataacatatataaagacatcagaatgataggaatatcacgcaatgttgaaaaacatactagtaacaggtttaatataaatgtagaacaaataaagttgaacaccatttcatatttgacaatgtttcctatatgattttaacatgccaaatacaccgaatatgtctgtttttggcttcaggggacttaatatcaaaaaagataatgaggtcaacaagactgaatttagaatttgattttggaaagtttgattttcttcagtccccactttgaaacttaaaacaagtttcatatattaaaagccaaggtaatagctttggagagatttgggttacgggtatttagatagagacagatattgtggtggaaaaacaaattacaatcaccaaacaggtcattgtaaataagtcattcatttaggcaaagtgataactcaaagatttcaaaaagtaaaaaccttcactctttgagagagaggactcactttcacaaacaataagacctaataaagacagcatgaggccaactaaatttgtctctcttccccaacccatctttttttttttttttttgcagttttctcaaaaggtaagcaaaaatctttcactatctcttaatattaaaagaaaatcttattcaaaggaaatctaaattttaccattgcatagcttattattaatgctaaagttagcattaataaaatcttacaaacaaatctatttaattagttgtaacacaaggtagagatttttataaatcttttacaacattttataattttccataaaagagcagattagtattctaataaaaccctgatattttgacataagggcctagattctggccctgaattagtatgctttttattttaaggtttaatttatgcaaaaactaaataatccctttcaaattttagccagctggcttatatatagaattccttttacaagatcaatgatcaatctttcataaacctttagttttgtccacatttttaacttaaaacaatctttaaaatctaaaaaatttttttttgttcttttgagaccgaatctcactctgtcagccaggctggagtgcctgcctccagggttcaagtgattctcctgcctcagcctcctgagtagctgggattacagctgtgcatgaccacacccgcctaatttttgtatttttagtagagacggggtttcaccacgttggtcaggatggtctcgaacccctgacctcgtgatctgcccaccttggcttcccaaagtgctgggatgacaggcatgaaccaatgcacccagcctgtaattttttaaataaataagactagcttaatattcgtttcataaaaacagctaaatcctgagctatctgtaaaaaaatacgtttaacattaaggtttatacttgggtgaacacctgatattcacaggctataaaatagttagcaaggaaataactttaaatgtgactagttttgtctaatgtctcagttctcaaagcaatctaggtaaactgctaacaatgaataaattgaacaaatataagtgggatggataaatgcttgttggttaacttttatgtaatttaaaatcttaaacttattttggattaaagaacagctactcattaatagtttggctcatttccaattaagtagagatatggagaaacatgcctaaaaattatagagtgatttcatctataaagtactgatacctgatatgcagtttaggatttcatgtttcctaggtttaaggtcactaaaaataaaaattccacttaatatataattctgtttataaagtctgtcaaagacatgtttttgatgaaaaaagaaaataatataagacaaatagaaaaatgtattctcattaaaaagaaaatcacttttgtctaattcaaaggttatttatgaaacaaagtaaaaaggaaccagtaaataggggaaagagatgtaaagaaagttatagatataaagatgtatttgtgataagagagattaaaaagaaaagagaattattttgtatgataaataattttatatttttttctgaagtaagatgactggtaatctaagaaagagggaaatttaggatacaatggaaagtccaatcatgttggaagtggcatgtgtataatcaaattcataaaagaaattttgtttgtggttatgttggctttaattaaaattgaattatttatagtcgtctttctggaaactgagctttgaccaaaaaaaccacaatacaaaaccaaaacattggttagagcaataagggtttcttaaagtattgatttgctcttcattaaattccaagaagttttaatgtttaatgctatttttttctttttgaaacttctcagatatctcagaagttcaacttctggtgtatcctgctgcttttagccttttcttcatttgagaatagctgagatcttaactctctctttcaactttttgttggctctggcaaattttttttttcttcaggtctaaatctgttgctatagcctgatgctgaagtgtttcgtcttaaagcttcaaaaaaagcaatgttttcttccattatcacttaattctgtgctgttggcttttcctcctatgagtaagggccagaaaccctctgaaggattactgaaaaatcaactcacaaaagatagatcagttgaagagaaggcatgcagatttgtttggagtgtgtatgctggagtcttcagaataaagaccccaacacacaggggaaattgtcctttttatgcttaggtgcgacaaggtatgggcacatataaaaatatgattgaggcaaaaggcataacctaatgccaatggactggggaaacccagcaagacctgtctgtctagattcctcttgacctctctgagcatgtactccttccttctgggtatagggatcttatgacctacaatccaacaaggtaagttggataatttctttatggctaattcatacacagaaagggagagggcaaattagagtaatatttttagactttgtggctggttttgagaaaaaggagttctgatttctaggttctgccttggggaaagagattctagtttgtttggccagctttgagggatattgggactgagagataggaggccaggagaaggccaaagaaaaactgacttctgaagcttttaccttggggcactgtcttctgagctacaacgtgtatttaaagtcttcgacgaaaccatgaaacttcctatgctggtactaagagtcacgtattcccctgctcaagttactagtaaattctttgttagtattgttcttcagtgggttctccccagtagctttgggttgatatcctcccagcttctagccttgcagcataatacatgtgcttagatttcagcaaaaatgcctcaatagagtttcaatgatctacttaggtcacatgccacccatcagccaagctttggtcctagagctgttgtgtttgaagtggtcctacatagtttatgtgacagcccatagagctcaatatgggcagcctattgtccacacagtagctagacagatcactttgattaggcgaacactgttccgcttataacccttctctcatcctgtctcactttgatttctcatctcgtttagaatagaagccaaaatataagcattggctccaaggccactgttagttgtcagatatcatctcaatccattccccttctcactgactgtgctccagtcaccctggcttcctggatgtttcctcaacacttgggacaggttccagctaaggatcctggtactttctgttccactgcatggaacaatcctcctcacacatatccacagaacttattctctagcatccttaaagtcttagtgagcctttctttaaccaccttgtttgaattcagtgctctccctgtgcacccactaacccctctttttgttttcaccaggcacttaccacaatctaacagactgcatgttttatccatttattcagtttcctatttgtgtcccttcaactcccattaaaatataatatttttgagggcaagcaagtactagaacaataggaaacacatcaagagtattctgtaaactatttcttgaatcaatcagtgaatgaatgaattaatcaatatattttttgagtgaggagctttgtgttaggtacagctaaatgggaaatcaagtgggtcatgtaccatgaataccatatactctactgtataattctcctgcttatatcagaaactgtttataagcctattataattgataccaattggaatctcttttttactcatcaccaagaacaccacaaacaagttgtttaccatttggctccttatttaatctggatttccaactcctcatgcttaaaagacggaagatacaataatactttccttacagggttctgagactactaagagaacttatgcatgtaaaagggattcatgcagtagaaatactaacaaaagaattactatgacagatacttataaccattgtgtttttacgtatttaaaatacgttatacctataattagtcacacgaggaaatcaaatgctaaagtatgatatgtttttatgttttgtttttcttgcttaggggatcatggacattgaagcatattttgaaagaattggctataagaactctaggaacaaattggacttggaaacattaactgacattcttgagcaccagatccgggctgttccctttgagaaccttaacatgcattgtgggcaagccatggagttgggcttagaggctatttttgatcacattgtaagaagaaaccggggtgggtggtgtctccaggtcaatcaacttctgtactgggctctgaccacaatcggttttcagaccacaatgttaggagggtatttttacatccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacctggaccaaatcaggagagagcagtatattacaaacaaagaatttcttaattctcatctcctgccaaagaagaaacaccaaaaaatatacttatttacgcttgaacctcgaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctgagaaatatatttaagatttccttggggagaaatctcgtgcccaaacctggtgatggatcccttactatttagaataaggaacaaaataaacccttgtgtatgtatcacccaactcactaattatcaacttatgtgctatcagatatcctctctaccctcacgttattttgaagaaaatcctaaacatcaaatactttcatccataaaaatgtcagcatttattaaaaaacaataactttttaaagaaacataaggacacattttcaaattaataaaaataaaggcattttaaggatggcctgtgattatcttgggaagcagagtgattcatgctagaaaacatttaatattgatttattgttgaattcatagtaaatttttactggtaaatgaataaagaatattgtggaaaaactcactgtctctaaagtttatgaaaacattgttggctaatatattgtgaatcaaagtttttctttagacgacttaggatattatggggctaggcattttttcctcaatagagtcttcctctcatcctctttcttgtctcctagttacattcttttacttccatccatactttgccacaagagaaggaacatgagctttattgtgtagatctgatttgaaatcctgtggacacggggtgaattacttttaaaatctgtggctctgattcctcaaagataaaatgcaaataatatttatataattcactgccagatataaattttcaaaactatttgttatatggatgaataacatcattaatattgtggttgctgggccagcatttgccaaaagttctccttccattttgctttattttcctgtaacttgaaattctggtcctactgtcatctgcctgcttcttccttaattaaatattgataggatatcagatgtctcggatctgagagtgtgccttgtgattcaaaatctgaatctttacttatccataactcagattttctgtttgtaaattccagtatcagggctatagtttaaactgcagatttgttcttaacactattctccctcttcgactcgtgatgactataataatcttaagagaaaagcagacattagaatgaataaatattcattaggagaataaattacattgaagcatcagtattttaggcagcagtgtaatagttgggagatactggtgagtgtagatatcctaggaagaggtggataggagatctggcctcagtgggaaggacaaatgaaagacatatagcaatatttgagagcttgtcatctttcttacctattagccttgttcagctctcctgctatcttgttgcaatgccaggtcaccactggtgctcctaggcagacccaagtttctcacattctgagcaagatcacatcacaggaggatgtggtggcaaaaaaacaaaaatgaaaacaaaacaatcaaacaaaaaaccagataaaaatgtggctcaagtatgatgatacagttgtatacaaatgaatcaagtaaattatattgcacaacgagtatcctgaaacctaattcagttgttttgttcttgattatatatagcacataatggaaaaagcaaaaatatgttcagagagattcagaaatacacaatttcgcttccaggttgaagccttctgcccttattatgcaatgttaccttttctctaatgaaatctaaatgagtgaagaagaatctcaccaattgatttggccagagattttaaggtgcctcttaattgtttgtgtttgtccaaacccatgtctctgtttttggtggccccctggagactaggatgtggcatatcttggtagaactctgagataagtaaggtagaaaccaaaccttctagacgtaactgggaaggtaggatgttggatgtatattccagttcttctattttc

SEQ ID NO: 2 - DNA, 11754 bp, Homo sapiens
ctcccgggctggcagcagggccccagcggcaccatgtctgccctcggagtcaccgtggccctgctggtgtgggcggccttcctcctgctggtgtccatgtggaggcaggtgcacagcagctggaatctgcccccaggccctttcccgcttcccatcatcgggaacctcttccagttggaattgaagaatattcccaagtccttcacccgggtaagagaaatagtgttgattttagggagaataactcagcaattggatctggtatgtgtgtattcaactcatttgcagacaaattgtggttgttcaataccagcctgttgtgaattacctgaattgatagcatcctggagcgacactcaaaatgtgtcgcctgtggtgcagctggagcccggagcctgcgtgccaggccccggaggcccccgccggtgccttgtcctggggctgatgatggggaggccggcgaggccgggctgctgcgacgccaggataaccgggctggcggccagatgcgcactcgctgggcgtccgcctgtgtttgccaaagcacgagttgaaacgtgaagtgttgggccagcccgtgtggcaccaatacctgccgcctacgactgttgtgaacactgaatgggccaacaaacctaaacgttaaatgaactgataacgccgtcagcacggagcaggcgctgggtgtttgcgctcttgcgcgtgcgctctgtggggcgcaggctgacggcgggcgggggtcgcctgctccagctcgggctcccgcgccagaaccgggtccagaaccttgattccggaagcgggcaacggggtggttggtgggcgcgcctgagggaagggacgtgaggagccggagtccgcggagttgccgcggagttgtccgcggagtccaggcgggtggggagcagagcagctggaaccccccgagcgccctgcagacgcagcagcctcttgaggggagggtctcccccacctcgggctggacaaagacagcttttccccacgtccctctgggttctctagagcaacagcaatacccgcccggcaggggtgtggcttagagccccgcacctcctcgccgcgcggcgggcctgacttctagccacgggtctccgcagttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggtgagtccgcgtccctggcacggagcggggggtgcataacacgccccgggacagttacgggcgctagccacgtcggcgatggccaaataataaactaacagtaatattatagtaacagcatccgaaggatgagatcaggattagggcgatggcccccgcgcgttgcctgcggagcgaggcgcactgagtcgcccaggaatccggcctctcggcgactgtgcgggagagtttatggggatgggcggggctgcttctgagcaggagtcgccgcccccacccccaccgttccgcctctgggcccgcaggctcctcccgggagcgctttcccctcctgttcaaccgccggggtacaggtggcttcgtccaccgaggtcccctcacccacgctgaggcgtcggaagctgcggacactgctcgcttcagggctttgctcagctgcagctggtgacctccagagagggagtctctgatgtcccgctggggtggatgtcctgagaccgggaagggggaagagacccactgaaatcctatctcccagcctcacctctgctgtctcctccacgcttcctgtctccagagccccgagttcagcataagcagaaagcggcctgttccctctctagggagaggagggttgcggtctggaggtctggctcgtctttatctgcgcattctcccagcctcctggcttcagacctcagcgaggcggcggctgccggccggctctcctcttcctgcctgcagacctggcctgctgcttctttctccttcctccctccctgcctgccctgcggtttcaaagtagattagaaataacagtgtcccacatggaagcctctacttcttcctgggtcaactttgatgacgaggctccagaaaacctttgcaatgctgtgtggaatttttaaatcggtgagctcgtgctcttgccctatttatttgtccagcgtacatttctgaacattgtgaacgtcgaatgggccaacaaatctaaaaattaaatgagctgataaagaacgccgtcagcacagagcagacgctgggtgttcgcgctcttgagcgtgcgctctgcggggcgcgggctggtggcgggcgggggtcgccggctccagctcaggttcccgcgccaggaccgcgtccagaaccttgtctccggaagcgggcaacggggtggttgtatcacaattagtggcatttggttttccttcttctgcattgtgggttttacttctctggggttgccaaaaacaaaattaaccatctcagtccttgtcgttaacgcaggagaagcattactggaggaggctctggggttctgtggttgaggagctcagttctggttccggggagcccttatctgccacccacgggtccaaggcacagtcggaggcagcagggaggggagcggaattcacatcaacacagatggggctcaaggggactttgctgcctctgcctggagggtctaaagtttcattttcatatgacccgcagggcgcagactggcggaaaattagcagagccctgggcatgggctgcacctggcccttaagggacaatgatggaaatattccttattagcacaatactgagcacaggctgtgtgataatgtgtcaagggaactgcagacatcctttcagaaaaagttcataaaacggagaaagtttggttcccaacctagatttttaacctgttgaactctgtctaaatgggtcatctcgggatgtcctccactcaacatgaccacgtctgcccctctgtcccacctgtctcctcagtccttcctccccacctttcaggatgaaatgaaaccctcagtccagctgcacccctgccccacccacctcatctcatgtgccctcccgcccctctcaggccggacagccttgcttctggaacacacgagcacagcttcaccaggcactttctgagcaccctgcaggcgcctcccaggagtggtcagtggtcaatcagctaatgaagctgcataggacatgacccttgtttaccgcagaatgcccagagctggcaggatgtcttatatgcaggaagtacccaaaatgtatttattgaggaagtgatgatggataagaggaagacggagagcgagggagagaggggctaggggcccctgcggtgtaaagggggtgtggctgggagtgtgcaggggaacagggatcatttcaaggttcctatctgggagaaaataaaaaggtttacagttagttgagataagcgtgggaatatgcgaacatttttaaagaataaaaagtttagctttaaatttgttgattccaaatgtgttcatactctcgggaggatccatcaagcaactcttgggaggagagacagggcagggccaggccttgacagctcagaagggtgcagtagggacagttcttggttttcccagctctgatgctttgcacagtcacttgtgtgacctgcaagattttagtgaagaaacttgctgtggagtcggaaagctgcaagttgaggtgtgtgtggtgtgagggttaaaaatctgtgagaacagaatgaatggcttttcaagaatgttgtcgatagataggaaagaggtgggaggtgttcttggagtggccatatgtggttttatgtagcatggggaagactcagcagaaaggaaaaagaaagaaggtaaattgacagcatgaagtagagcacccaggagaggctacatgtgatgaagaaaccacagtgcagactgtgaggaccccagaaaggctcctccccaaaacctgaccagtggccggtgctggcagctcccaggctgggacacccctctgtctctctgtccctctgccccctctgtcacttctttatacacctgtaaatcctgccctgctctccaaggccctctgtagcccatttctccccaaaatgggtatttagaataaccttctgctggcccctctgccttaggaatcatttttaataatggacctacctggaaggacatccggcggttttccctgaccaccctccggaactatgggatggggaaacagggcaatgagagccggatccagagggaggcccacttcctgctggaagcactcaggaagacccaaggtgcgtatctgctgcctagcagggcccagtcctcttgcagaccagcggtgtggggagccctggctgggactcctagactgcatctgaaccacagggacctacggacaaggagagggtctcgtgagtccccagatactgcattttacaactctaggttccagctacacagttcagggagcaagggtggccattaaacacgtgacttgtatcctaaatactgttgaaaagcaaaggaaactcaaacaggttcagacattcactatctttcgtaaactggcagttttcagggcaccttctcacaggccttggtgaacctcagtgggtgactgagcaggtggaggagtctcctcacccccatcttctggttgccctgactgcctgttttgtaggccagcctttcgaccccaccttcctcatcggctgcgcgccctgcaacgtcatagccgacatcctcttccgcaagcattttgactacaatgatgagaagtttctaaggctgatgtatttgtttaatgagaacttccacctactcagcactccctggctccaggtgaagccactttcctctttcatcagtcatcaactgtagagtttacgttagaaaaagaaggaaaatttgggttatatgtgatagacaggactgcaaaagccaaacaacatagcttcgaggggtgtttgattagacagcccaaatattcctcccagagacatctctggggccccacgcaccccctttcctaacgtcaggatgtgtatcgacctgtgtgtgcacatttgccatgcagagtttgcactgctgaggagaatggtgcccaagaaggacactgttgacccaaaatattccaaataaacaatgattacagccacaaattcaggtttggagaaagttgttggtccaacacacacaattatgttgcatccagaaaaaagtagtaaaatatttttttccctctctagctttacaataattttcccagctttctacactacttgcctggaagccacagaaaagtcataaaaaatgtggctgaagtaaaagagtatgtgtctgaaagggtgaaggagcaccatcaatctctggaccccaactgtccccgggacctcaccgactgcctgctcgtggaaatggagaaggtaggctcggccctcccatgatgtgggctctccggggtgggcagagaatgcacaatttcagatttacagagtgagctgcacttgctggtgtccagacctcccaccgcagcatgctctgagtttcatacacacactcttggcttcagcatgaccactggacgcaagtcagcctgcctggctgccaagctggcctggggctttggggtcacatgggcgggacgcttagctctctccaggccctgctgctcaaccctttctagtctgcagactttgagaattgcattttgtctgaggagaagccctcagccttccttgtgggcatgcactccccaactgtgcgcagctgcaggacttccaggcctccccagcttcatccacctgcaggtgctcaggatcctgatcccctgcccccttcccaccttggtgaaacttcttgtatccttgtcttgtcctttcctatggcttgtggctcaagaacaaatgtggagcccacactgatttcccaggactgtctgagcatcttctccaccagtttggcccctcgtggcagcagacactagccctgtagcaggaggggttagcaggagccgtttagctcctgcctgagctatgaccaaggtcagggggatctcacctctcccaggatggccctcatgctgtggagggagacagagccctggcctgccctcagcagatttctgggagcctcagtttccctggctgtgaggtggagatgactctgtctgtcacagctccaagtcacagttccactgggagagcctcttggacactgtctcctgtgtccctgtggagctgggaggtggctggttctgtgctgaaaggagacaagcagccccttctcctccggtctgtctccggtatcacaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaaggtaggcaagtgactgaagggacaccgtgcgtgcggctgcatctccctggatggccagccttgcacattttaggctgcagctttctgtctgaagctgcttgttaaccctcatggtgatgtggtgagatggctagatgcactgctgtgaggggaggtgttatggtctgtgctgaacacctggtactcttgcacactggttggtccataccccactaagacacccctggttgcagaaaagaacatcccaacaccagagtggagagaggtggcagggtctgcattctgctccataaataacctctttatgacagagaagataatgtcccagttccccccaagtaagacctggtcttctaggcagagcaggtggggaggttggagctggaggggagggtccttgctggggcgtcttcctcaaatgcggacgtgaggagggaagtccaggaagaagcagctacagctgccctggacccttgttccttccacagggctcctcccagcggcacctggggcagctgggactctgtgcctggaggaggtgtgaaaggtctgggtctaggtgggcagagggtcatgccctgagaaacacccatctgggccaagtagaggtgatgtgagggcacccgcatgcaaacaggccagtcagggttgggtccaagtaaaggggaggaaagggagctgcagcctggctggagagtgccggggggcccagagcccctgcctctcgctgggctggaaacagggctgggcagcctctgcccgaggcagttcacagcctgagtggtgtgtgccgccctcctcctgaagctgctgctaatggtcacttgtggtcttaaggctcgtcagttcctgaaagcaggtattataggctatgaagttatttcccccaagaaagtcgacatgtgatggatccagggtcagaccctgggcttttcttgttctttccttcttcttcttctttttatttatttatttttttttttgaggggacagggtctcactctgttgcccaggctggagtgcggtgatgcaatcatggctcattgtagcttctacctattgggctcaagcgatcctcccacctcagcctcccaagtaactgggccacaggtgcacaccaccacacccagctgattaaaaatttaaaaaaattattttggctgggcacagtggctcatacctgtaatcctggcactttgggaggctgaggcaggcgatcacgaggtcaggagttcgagaccttcctggccaacatgatgaaaccctgtctctcctaaaatacaaaaaagtagccgggtgtggtggcacgcgcctatagtcacagctactcaggaggctgaggcaggagaatcgcttcaacctcagaggcacagggtgcagtgatccgagattgcaccccactgcactctagcctgacaacagagcaagaatcagtctaaaaaaaaaattgtagagacaagttgttactatgttttgtaggctggtcttgaactcctgggctcaagtcatcctcctgccttggcctcccaaagtgctggggttacaggtgtggccaccgtgccccatccctggcctttgctttttcaatcacatggaaatgtgaagggtgaaggagccaaaagtttagggaaggaatcattgtatggatctgcagtgattataagagaactttcgactactctgcactaggggaaccatggaatcaaaaaatgttttaaattattatttatgaggaggttccaatatagacaaaaggaaaataaatatgattgacatgtatatatccattgccaaattgaacgtttattaacattttgcgatacttccatcagagctcttaaaaagaaaatgtgttacagagccagccaaagtctacctcctcacatctccccacctctctcaccagaaatggcttcagaattgctgtgtggctttgcacttttaacagttgttaattatcagcacagtattcatattattgctgtatgtgtttaatattttacctgggtactgtacataacattttgcagcttggttttttcactcaacatatgatgatgttccatgggaactccaaacacggggaggctaggcgacttgctcaaggcagctgttacctctgtcagaaagacagaggctttcagattcaagaagtagaccctgcatgtctgattctgttctgtaaacccccttcatactcagaagcatgcaataaacaagcctggggtaattatcaatgcaaaggttaccctcccagaagaaatttccaaaacactttcattattctctgctcttgacatgaagagaactgaataagccatcatcaactgagataatggatgccaaaacatccagtaaataacctcatagagcttagctctcactaagtttttggagcattttccagtaattcaaaggacctggggaaccttaagcactgcttaggatgctccataaacatcttctgcgtgggtaggggagtggatggatggctggatgggtgggtggatggacggacggatggatggatggatggatggatggatggttggatggatgggtgggtggatggatggatgggtcaatggatgtgtggatggatggaagggtgggtggatgggtggatggttggatgtatagatgggtggatgggtggatgcttggatggatgcgtgggtaggtggatggagggatggctggatggatgggtgggtgggtgggtgggtggatggatgggtggatgggtggttgggtggatggatggataggagggtggataaatgggtggatggatggatgggagggtggataaatgggtggatgggtggatggttggatggatgggtgggtgggtggatggatgggtggttggatggatgggtgggtgggtggatgggagggtggatggatgggtggttggatggatgggtgggtgggtggatgggagggtggatggctggctggctgggtgggtgggtggatggatgcatgggtggatggatggaggatggatggatggatggaggggtgtatagatggaggggtggatggatgtgtaggtgggcagatggataaaagcgtgattgaatagatgggtggatgatgggtggatgcccaactggccaggaaccaatccctgaaatttgtcccattcatatcttggcagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaaggttaagcaatgagcctgcagcacacagcatgaacaccatcctatcagctaatcgccttcctgccagggagcaggatgggggccccaagacccttccctttggcaggggtcactgaggggaagggctggccccactcccaccctgtgggatactgcatctccaggagtgctcacattggcctggtgaccagagaggtggaggaaatctggaaaagagcctcagcagatagtgcctgggactgtagtgaattctaatgccaggaacaaactatcacaaccagccctggggttaatcctgtgagaagattagggctttcatcttcatttagacctgacccctgactgctttctatctaatccttcactaagcaactccttcaactggaaatatactatcctatatagcataatattcaaaactacattcttcactgggggtttccagatgaaagcccacattttgttaacatgactcactgagacagtctttgtttctcctagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggtgagaaagatcagaggcagtaccttcccttgaggagcagcccacactcctcatctcccctccacatgtgctctgccctcgtcccaggcacccactgacaccccaaacctcactgtgtgccctgtttctattgacaacatgacccaaatgtgctcttccctgttcagagaagttacataacatcttttagcagcaatcctgggaatgaagtgttgttgtaggtggattttttttttcccaaagactagacattttacatcattcattgctaaattttgtttctattttaacaagacttagtgaaaagctctcaaagccatattacccaattctccctaattttaaaccagagctactaaacaaaacctaacctttggttacctagaatcatcacaggaagcatcaaagccttcctgggatgtgactcagtgattttctttgaggcacttgtcctccttcccagggcctcatcttagggattgttgtgggaagatcatacaaccaactccatacttttcacacccagtgctggagccccagcttctaacagggcactatttccctcctgtaggcatcactgatgagcactgggggtgccttctttactgggcagacacggtcttcccaacttaacaccggtttttgcagttgagctctggataattgagattgtatgaagactggtccccgaattagtcagtgttgctggtatccttccactcaagtacattttgtgcttcttttaataggcagagaagggtgagtcctgccttgtgatggccgtttgcccacagcctcctcctccccgcttcccctagtctcactgttaacagtgtcgtgtctctgaaactccctcagtgtctcatcaataccattgttacttctaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattatttt

SEQ ID NO: 3 - DNA, 104550 bp, Homo sapiens
gagatgacgaataggggaagtttaatacagtgacaataccgatactcatcaagactagaaaattatagttgccagatcactagaattattgactattctggtctggctacttagctgcagatggaagacagccaagaatcacagggagaggaagcttgaccaggaggccatgagctgatccctgaggtgactatggttgccattctatgtttaccattctttatgcctgcccctcctctgctgctcttccttcaggtcttctgccttccctctcttctgctattgtgctaagtctatcgattcctctgctcagatccacagacgtgacatatggagggtcaaaaccttccccattgagatccatcttccttccatgtttcagggagcttccaagccaccagggaccactgatttccagaagcccctggtcctcctgagagaatgaggagcaaatccaagcactgggcagaccctgcaagagtgccgcgccttcacccttctctgcatatcctgggctcagggacccggggggagttggcagtggctaacaagttccgttccatctgctgcttctggcagagtggacctggaacctggggcccttctctaggtctccccgagccaaactatcaaatgaatgacagctaagattctatcgatttcccaccgtctggttcttttctttccctgtgacatttttacactctgcattttagtttgggtttgcagaatttcacaagattcttgttcgatttccttcctcaaagcactttattgtgattgtaacattaaaaacagagaaacaaaccacgtccatgagtttttttaagacaggcctgaaagccttggagcctgctgtgtcacactacacgctgtttcatcttctactctcctcactctgattcttctaaggccaaactttctcactgctgctgccaaccctgcgttagtgagactcaatgagatggtttttcctctgccccccacgcctatacagtgtaaaggggccatcctggatggtgtccagctaggaaagcccagcctctgctctctattgctgtcagaagattctagaatcacaggggcttgggttcgagtcttgattataccttaccagccatgtgactttttgtgacaacttctctgagtccagccagcttctttatttattaaaaaatgtatctctctcatagggattttttgaaaagtaaatgagataaatatggaatgcctggcacatattacactcttaataaacttttgtcctcctcaaactgtctctcactatggaggtgattttgccttggccagcaggctggaggccttctggggaagcattgaaagcaagaacttgtagtctcctaaaagagctttaagctacttactagatcaataaagagatatgaatgtcttaatttacccaatcctcataaaatgcaccaaaataaagctaatgatacaagaaatccccttaaaattctcaacaaggcttgctcagtgtgctgagaggcaactgtgttattgtagctcatgagataaaatttatgtttcagaaaatgctaaatattatgtcatgaactttcagaattactcacaaatagccaaaatctcaaatattaacccataaatgccaaacatcaacttactttcaaagctctggagaaaggggcagacattttccaaaagtgtgaaaaagagtggaaggaaatgtcccatcagtagaagacaatattggccaggcatggatcaaaccaccagtaagtgattcctgcagtagggaagactctatggagaaagtcagtccagtttccatttcctctccttgagtgttgggaaaactaaacttactctagagaacagatctgaggttgtcagcttgccctctgtccctgattgctttggggaatatgctccttttgggggttatatgcctagggatgcaaacagtagtttaatggacttctttttgcaggagaaaagaggttcttttctttttaaaagtaaaacaaagaaaataacagcattaacgatactgagttcctgtggagagttccagtacaagaacaaatttagaacaggaacagcaggtgatcttcctgtagccttttgtgctctgaattgacttcttagagtccattgtaataaaactaggaagttggcccaagcgatctggaggaagtgctcaaattttcatattacacttatttcaaatatatcaatatatcagcctgcattttgcatctgtgttaggccattcttgcatcgctacaaagaaatacctgagactgggtaattgataaagaaaagaggcttaattggcttatgtttcagctctgcaggaagcatggtgacagcatgtgctcagcttctagagaggcctcaggaagcttttaatcatagcagaaggctaaggggagcaggcacgacacatggtgagagtgggagcaacagagagagagggagtctccacacacttttaaaaagctggatctcataactcactcgctatcatgaagacagcaccaagccgtgagagatctgcccccatgacctaaacacctctcaccaggctccaccttcaacaccgaagattacatttcaacatgagatttgggtgggacaattatctaaatcatactagcatccttgctctggggagtggtgaagtttaggagggtaagtaaagagcttgggttcagtttttataatgttggctcatggtttatttctaagatacagtaaggatacactttaagcctctttgtccctagagtttactcagtgcccagcatagtgcctgacatatgtcatatgacggagccttgttatggttgatcagtaaatggatgtgtaaatattgcctagtgtagaagttgctctggaatccatccttaaaacatagtatgggaatggggacacttaaggttgaagacctggggatgaaaataagttctcctaaggacctaacacctgtttgtggtatgaatggatgaaaaagggaagtgatcaaatgaatggttccactcaggtgtggattagagccacaagacagaaaagactcagatgaactaccaaatatttgacctaggagggactttctagacagcatcaagttcaactctccaacatcaggtaggaggaaaccctgaatagttaagtagctagttcaaagtcatccagcaagttagtgtcaggatcaagattcaaatgaaggtctccagtctcctagttgggtgatctttccactacactgcctttgcaacatgaacgggatggcttgaaaataagaaacagaaataaaagaaacactttataaaaaaagaaaagaaaagaaaataagttctcctgaggagcctatgaaccccagtgataccatttgcacacacagctttgcatgagggccaagccagacatttgagctatgctcctttgcgtttaactaaaacctggttctgatatcgttttcattcattcatttgttattcaacaaatacttcttgaatataatagtatgccaaccatgtccaggcagagtttagcattgaataaaacagataatatctctgcctttgtggaatttattgtattagtgaatgtaatactagaggcaataatagataaacccagaaatctcagtgagcaagcacaatagaaatttattttttgttcctgtaaagtcaataagcaacaagggtagggggaaatgttcagctcattatatttatttaagcacttgagtttattgaaagctctgctgtcttcaatttgtgacttccaacgtcaccctggtcattgacatcagttgtcagacagtagaaaagacagaatgggagcctgggcattgtacacatcagttttacccatattacattcatcaatattcaatcatgtggctttagccacatataagggagctgcaaaatgtagctcctggctggatagtgacttcccagcagaaactcttcattagagaaggagagcatgaatatttggtgggaagattgacattcctgccacatttacgttctgctgggtgcaggaagacagacattaaacatataagtgagtgcactatgtatcatttcagatgataatagtaaaaataaatcaggggaaggaattaagggtgcaaatagtcataataataattttctccagtggctactcattggtctatattgtctacagatcacatgtgagttgatccttgaataagtgaagaggtaaggacttactttcctgaattgtgcaagagcagctcagagtggaaagagaaactttactttgtccagctagccaccaaacccacatggtcgttgccacacgtgactctgtggtgacagactttattgagtgaaaaatacattataaaaaacataaggatgatgcagacagattgaagtgggtttacatttgcttcgagggccttttctattccctgggcctttgtgtatttgcagtgaagacccaggctgaagtttacatgcttttagttttgactgtgtccatttttttttataatctcatcttcctaagaggattctcccagatgtgtatttgcttgattgattgatttcctctatagtaatgaagtaatgtgacctttatcttaaggaatactagctggttaactaacaaaagtttagttagaatggaactggtaaacgaagttaaatgatgctgagaatctatgaaatgacggaagaaacttctgtgtttagtaaaaggcagacgcttagatatttgtgaaaacataactcatcccttgcactgtggacacaattcaacaaacattttttgatgcttctatagatctggctctatgctaggcaccagtgaaacagtggtaaacactgttctgctcctaatctcaggggcccactatctaggcaagagagaaagacactttaccaaccagaaagctggatttcagccagactaagaagggccataaatgctatgctaacaatcctggttttactagtgaagaaccatcagagagataacttttctgctgggaaatgggtgacggatgtagaaactggcatttgcttgtcgtgcgacagttggcaagtcaacctctctgactctcacttgcttttgtctgaaaaaggaagataaaaataacttccttacttacctcacagggctattataaggatcaaatgaaatcaagatgtgtgaaagaaatgttattcatagtcaagaactattattaggattacttaataatatagatgtctgagagaacaaaggagtattgggtgttgagtagtaatgctctgagaatatggcaaaaatctttcatccagagtggagcatatgtaggagtgaaaccagtcttggttaaaaaaatctgaacaatggaatatattgaaaaaacattttctttcttttaaagtaaactttgttgtaaataaagaaaatgaacaagaatgcccttaggttacagacaagaatgtacatctggttgaaaagaaaaaggacggagcagactgggaagaagctgctgcagaggcaggataaaggccagcagtgacttctaaagagagctagagacacagccagggcacagaaggccagggcatgaattcaaaagcactaaagtctccacagatgctggtctagctctgaccagcctttcaggaaacactccatagtctttcttgaatcatcttcttccaggaagtacttggccctgagggagctcactcttcaaaaaataagttatgactcttggcagcatttttacttttcttatcttttatgatttcctgaaagtgtggctcccactgggaagaggtctgtgtccagaaggcctcagggcttatctggattttctatgaattttaaagcctcacatgccaatgcagtgtatattaaaagccaagatatctcttaggagaataaggtaagttaatgtaataaaatgtacatcacagttggtctattaccaggtggtaaaattccattacttcagccaaaatttcagaattcatcctaatgtgaaaagcatatacagtagatattcagtatggaaaaagttctgactttagatttttttcccttcatttatactattttgaaaagtaccaaagaagagactgatgtcagcaagatggtggaataagatttttcagcactcatccttttgcagaaacatcaatttaaacaaatatctatgcaaaaaaaaatgccttcacaagagctaagaataccagctgagagattatagcacctgggtacagcatagaaatgagaaaagatgcattgaagagggtaggaagaatagttttacattatctgtgttaccacttccccaaccctaggcagcacagtggagagagatatccttcacttggatatctctccaagatctccaaggagatatccagggacagggaattgagcactaaactttgccttggacccaacactgggtccactagagtaaaacccagagccaggcaggtgtccgtgggcccagactccaatccacttggggattaaacctccaagcccactctggtgccaagtgtgaccccacagcttcaggttccaccctagcaccctaagccagcccctgcacattcaggttcaaaacttatcccagcaccaggtcaatgcctacagacccagacatcaagcatcagtggaaataggctccaggcttgccctcaccaactcaggctccaggcctactgcccttggattcaatcaataggtctaccccagtggatcctgaccccaggcccactcacctgctgacctaggcatcaggccagtcttcctgaaggctccagcagaaagcccactctcagattataccagacaacctgccccaaatctctggacgggctgactggtgaaaaactttcccagacaaagccagtttgcaaatactggaataagtcactacttctttaaatgctcagatatcaacagaaggcagaaagaaacatgaaaaagcaagttgatagaacaccaccaaaagaataatctcccagtagctgaccccaaggaaatagagatataaaaactgcttgacaaggatttcaaaataattgttttaaggaagttcagccaacttcaagaaaatataaagaaattcaaagaaatcaggaaacacaatgcacaaccaaaatgggaaatttaacagtgatattgaaattatttaaaaagacagaaattatagagctgaaaaatacaatgaatgaaataaaaatgcaatagatagcatcaaaagcaatactgattaagcagaagaaagaatctgtgaactagaagacaggttatttgaaaacatacagccaaaggagaaaaaaaatgataaaaaggaattttaaaaaagcttatgggtcttatgggacaacctcaaaagtacaaattttgagtcataaaagctcaagatggagaagagagagacaatggaggtataaagcttatttaaagaaataatagcagaaaactttacaaatatggggaaagatataaatacacaggtaaagagggtcaaatgtctccaataaggttcaattaaacaaaactataccaagacacattataatccaaccattaaaaaccaaagagaggatcctgaaagtagcaagaaaaaagaagcaaataacatatcagagagttccaatgaaattagcaatagatttatcagcagagacttacaggccagggatagtgggatgatatattcaggatgctgaagaaaaaactctgccaaccaaaattattgtacctcaaaagctgaaatgaaggagaaatgaaggagcgatgaagaaggagagataaagactttcccagacaaaaactggaaagaatgtttacatcgctaagcttgtcttacaagaaatgctaaaggaagttcttcaagctgaaagaaaagaacactaattagtaatacaaaaacatatgaaagtgtaaaactcagtggtaaaagtaaatacacagtcaaattcagaacactctaattctgtaatggtggtgtgtaaattgcttatatatttagtatgaaggttaagagaaaaaactattaaaaataatgactacaataatttgttaagagatacacaatataaaaagatgtaaattgcagcttcaaaaacaaaatgtgagggagaatggagcaaaagtttagggttttttatgcaatgaaagttaggttgttatcagctttaaatagcctgttataactgtaagaggttttatgtaagcttcgtggtaaccataagggaaaaacctacagtagatacacataagataaaaagtaagcaatcacagcataacactagagaaaatcacttaatcacaaagtaagaaaacaaaataggccaaaagcaacaaaagatctataaaacaaccagaaaacaataaaatggcagtagtaagtccttacctattaataattaccttaaaagtaaatagattaaattttttaatcaaaggacatagagtggctgaatggaaaaataaataaataaaacaagacccaactataagctgcatacaagagactcatctcacatttaggacacacatagactgcaagcgaagggatgggaaaaaatatccatgaaaatgaaaaccaaaaaagagcaggttggctatacatatatcagataaaataaactttaagtcaaaaactgtacaatgaggcaaaaaattattatatagtgaaaaggggctcaatttctcaagagaatatgacaattgtaaatatatatgcacccaacattgagacacctaaatataggtctaatcccagcactttgggaggccaaggtgggcagatcacctgaggtcaggagttcatgaccaacctggccaacatggtgaaaccccgtctctactaaaaatacaaaaattacctggggctaaatgggagtgcttcaatggtgggtctgtgagcaaagattgtgaacttgttcaagacaaatgagaaaaagagagtacttcttcctaggaaaaatttcatcagcctctccatgcctttgttataaacagacagaggccccatttaactggtctagagaattttggaaccaaacatgagggtggcacatgcctgtaatcccagctactcggggggctgaggcaggagaattacttgaaccagggagttggaggttgcagtgagctgagatcacgccactacattctagcctgggcgatagagctaggcttgtcaaataatacagaatttcagtcagatagaaggaataaattcaagagatctgttgtgcaacatggtcactgtaacttaataacgatgtatattttgaaaatctctacgacaatttaagtgtattcaccagaagaaatgataaatatgtgaggtaacatgtatgttattcagctcaatggagctatttaacaatgtatacatatttaaaaacatcatattatacatgataaatgtgtatgattttaatgttaattaaagaaataaagtcacagagaaagaaaatggtggcaaatactttgctaaataataagtaatcatactaattcaagtttttgtcaaatgtcagtatttggggtttcaactagttattacagagctgaaaattctgcatgacaacacaaacccttattgaaaatttatttcacttttatctttcaatgctccatagtaatagagaggaatttcacctctagttaagagaggggttgaagatttgctgagtggggagaagcacaccaacattagtttctcctgtggacaacagatttaatctatgttttatttcattcagctaagagttggtccttaccaagcaattatggccacctccgttttgtaaatgagaatactgatgccaagagagagatttttgccaaaggcggcacacagaagtggtggagctaacattcaagccagcctatggaactccaaagcctgagctctttcctcagcccgtccctttagcttttcctggagcattggctggggaatgcctaggaggaaggggagggcttgttaaattgggtagattagtgcatggaggaaaagcctctcaatcaaagaggtgtgagggtttggaagaattgactaattacaaaacatgccctaggctcagtctctctctctccccagctccttccttctttctttcagggcagaatttctccagcttggctctattgacattcattgttgttcattttattggggtgggactgtcttgtggtttgtaggatgtttagtggcattactggcctccatccattaaatgccagtagcatccctccccagttgtgacaataaaatatatccccagacattgccaagtggcttctggggtagggaatctctcagatttaacaaccggttttggtttcggttttgtttttgagatggagtctcactctgtcgcccaggctggagtgcagtggcatgatttcagctcactgcaacctccgcctcctaggttcaagtgattctcctgcctcagtctccagagtagctgggattacaggtggcccactgctcccagataatttctgtatttttagtagagatgaggttttaccatgttggccaggctggtcatgaattcctgacctcaggtgatccacccaccttggcctccgaaagtgctgggattataggtgtgagcctccatgcccagccaacaaccagttttatagtgaccaaccatcccagtttgccctggactgaaggatttactaggatctctgaggcatttgtcagggattcaaggctaaaactaggatagtccagggcaaactgggatatttggtcaaccaacaagtaaagtttcatctcctggttaaattaaggctgtggtgagagggggcattgtgttgattgtgtggttgtcctgtcagtcttcagtttctctaagctgttgctatatcaagtacatctgcctaatactcctttcttccccctcaggtagcctgctagggttgtaggggaggtgtcttacttaaggaaggctggctgagccactgtatgggtcattacaggacagaagaagaccagagtgaatgctggtctctgtgtgtttttttttttttttttttttttttttttgcgtgtgtgtgtgtgttgaactaaagaactggtaggggcagttgggagggtgctggactgcagagagccctttagtgctgctccagtggacgtgcacatctgtgtttccactgagagttaatggatattggatggggaaaggttttcagctatgtgttactgtagcctatgactatgtggtatgagcattttaatttatttggtgttttgaaatacttttatgagttactctgacaacttgtcaaggatattttcctgagagaaacaaataaaagacaaatctcaaaaatagtttgctattaggattttatttgttgagatgttgaagatgtcttttcaacagtgatacatgttttggttctttgcaaatttacaaggcactcccaaaatgtcatttgtccctcattcttctattcttgatatcattctatgatcctgtacaggatgaaagcaactgagttctgaaggtagtaagtaacatgctgaggtcaaatgccaggatttgagtcaggttcatggggctccatgtcctgtgacctctctgctccaccaggctgtgaaagaatgactcttgcattcatgcagtcagtctgtgttgctgtaaacctgaatggtaaaatgacaaatgtagacacagagggtgtgaatcagctctattagcagtgactctgagaatagtcctttccctcctctgtcttccccttctctgtcttgcagcaagtgggaggacgaagcaggtttcataagagcagcgcccaagacccacagtcgcctagtgccaagtcaacaaccttactgatatctggatgtagtttcctgtgctctttgacacaggtgtggagattgtaatttgctgtgtgtgattgtttgcacttccttgattatgtaaaccagtggcttttgccaagaactcttcatgccctgaagctctgagctggttccctcccattggtggaccttatttcattttattgggaggcgtaactttcaggtcacagagcagtgataactacctgccagtgtctcttaggagtgaggtacctggagttcggggaccccaacctgtgacaatgacagcagacaaattggttttctttgtgaatggcagaaaggtaagtaggagctgacctttggttttgtgtcccttctccctgggaggggaaatgtgtcctctggcagagccatatcttgttctctgtctttcctgtcccctcttacttgttctgtctttctctctctcgctttaaaggtgtcgctgcattttggtaacattagggaagaagaccttctcggggctagagcacagacaagatactcagaggccaaagaatgagggagagagaggcaagcagagaggcatctgtgagctagctgccaactctctgcagtagtggagattggcacagaatgagaaaggggagttgagttctaggtgagatttgaggtcatcaccacacttctatttctgtctttaatcaaatatatcctaaaatttacgttgctctctttacagagagaaggaacaggaatgtgttttcctgcttttatcaggatataagatgtacgcagcctttgattctaatatattatatatataggtttaacagccagtcctgattcactgtgaggtgacatatactgacaccttgtggcaaaccacaatttttttttcataaagggcatctcctgcattccccccactccccacccacacacagtgagttctacatgtaagctgcaaatctgttacctacttgaatttaccttattggtacacagggacttagggaaggtgacatgggtaccagcccttggacaagagtgagaatctttgcaaattttaatgtggtgcagagagagagggggcaagcatattacatggtcactagcctccatttgccctcctacaacactgctgagatgctcatacctgttcaacttcccacttaggaatgaaaaggatagtaaaagagtcccatagttgcacattgtaacttactgtgtggactgtgcgtgatgaatgtgcccaaagtgtcccccaaagaagggcaattttatttgtctttctatattggcatttttgcattgagttgagaaacttttgatgtaattttgagtgacttttggtataattttcaagaacaataatgtgcttcactggactcctaggtcttaaatttccaggatgtgtttgggtgactgtaaaagaaatttcatgaactgtttcaacgtcagtgctccaaagattgatggactctctttgagtggtgcctttgggaagtgctggggaatggggacagccatgttcttctgggttcagagtttcctctctgagctgcatccttttcagggttggcacctggtaccaacccaagaagtagggcaaagctttcaagtgtaagattttgcatccagcatttcaaacaacttctttccttcagcaattacattgtagaaatctaggaaaccattgtgcaattggctttctaggagagctacttacacaaagccatctctggaatttttattagtaatttttacattgggtaaacagctagattcaatgacgtccaaggtcatgtcttactctgaggttttattatttattaaaccaaaccattggttaacctgtaaaagtataagtttctgctcttcttcagggatggcttgctgttatgcactaaatctagaaagaagaatttcaaggtaaatctggcttggggaaaattattttcccttgcattttactttcactttaggagagtaattttaaatttttcgatggtaccatcacatcaaatcctattatttctagaagcttctactactgccagtttctgtgatgaatatccatgtttgtaatttataatgatgtagcatcttcagaaactgcaagaaaggtagaggggcaatgcagaaagtaattatgtgaaagcaatgctgtaaaaacaagaccaagcaagggaattcctgggtaaacaatggcagctgtgagaaagttgtcctcatttttattcagaagatattctagaccttttgttacaggaattgtgcaaagtagttaccaaagaagctgtaagaacttagtgactctttttttttattattatactttaagttctagggtacatgtggacaacgtgcaggcttgttacatatgtacacatgtgccatgttggtttgctgcacccattaactcgtcatttacattaggtatatctcctaatgctatccctcccctctccccccaccccacgacaggccccggtgtgtgatgttccccttcctgtgtccaagtgttctcattgttcaattcctacctatgagtgagaacatgcagtgtttggtttttaaatgtgagcctactcctctccttgttttgtaccactgtccaggactctggcttcactaaatggggtcctgcagaaggtgttcgatgcaagaagtcgaatacctctagggccttctgtggtggtgatgagccttgatgactattgttttcattcacatcttcttttgttctagagtcagagtcaaagggaaagagctgtccagtattttatttttattgtaatgacagcctttttgcaaggcagcattctagcttttctgagtagtcactctgtctagctgagactgtggcacctggaagaatctgggaggatctggcaggttgggtgcagccaaaggtgtccaggagtgatccccacaagaagtgagtgtcagctacagagatcgtcacggtgactttcactcactcactcactcactcactcacttactcatcattcacttgctcactccccatatagtcaacatttatcaagctccttctgaggcctcagacaacaggatatgcactgggggaataagatctccaagatcaggcatttgccattctggagcccatatactagttagagagatggattataattagacaaagtaataccttctgaaatgtatttcacagaaaactagcagcatgagatgctcttcttggatattcttaaaccacttagcacagtgcatggtatacagtaggtgtccaactagtgtggatggaataagtggatgaatttttaaaactttgagaaatcctgtagtaaataaattacttctctaactttgtattttacaattattttatatttaaagaaaaactgcaagaagaatacaaggaaatactaaataccctttatgtactttaccaactgttaatgttttgccacatttgctttattattctctctttctcactctggacacacacacagatacacatgtatgtattataagtacgtaaataattctatatattaatgtacataattaatataaatgaatatatagtataagatagatttctgaaccttttgacagtaagtttcagacatcatgttcctttagctcttatcctaagaataaatatattctttttttttttttttttcctgagacagggtttcactgtgtcgcccaggctggagtgcagtggcgcgatctcggctcactgcaaccttgccgcctgggttcaagcaattgtctgcctcagcctcctgagtagctgggattacaggcgcccaccaccgtgcctggctaagtttttgtatttttagtagagttggggttctaccatcttggccaggctgatgttgaactcctgacctgtgatccacccacctcggcctcccaaagtgctggtattacaggcgtgagccaccgcgcctggccagaataaatatattcttacaaaactgtagtccatttatagaaatcaagaaacttagctttggtgtaatactataaactttatgcaaatgtattccaattttcccaatactggcatttataactccccaaccccagcccggaatccaagattacgccttgcagttggttgccatgtctttagtctcctctaatctagaacagctcctcagctttcctttcatgacattggtgtttctgaaaagaacaggccagtttttttttagaatgttcctcacattgaattgtattatgctttcttgtaattagattcagagtctgcatttggggggcaggaataccacagaagtaatgtgtttttctcactgcattctgtcattatgtttgtttttttaacgtcagtttttgggtcttttttgtgatgcgaactttggtgacttggctaaggtgttatccaccacgtttcttcactgtaaggtaacttttctttacctttataattaatagaggctatgtgagaagataatttggaatgatacaaattcttgttcatcaaattttcacccactaatttgagtatccattgatattctttttttaaataatttttattgtggtaaaatacataaaatttaccatcttaatcatttttatgtgtacagttcagtggtattacatacattcataatattgggcaaccatcatcaccgtccacctccataacttctttcatcttttaagacaaactctataaatttactaaataataactccctattctacctttctcccagtccctggaaaccatcattttttctgtctctatgaatttgactaaactacctcacttaaggggaatcatacagtatgtctttttgtgactggcttatttcatttagcataatgttcacctatgttgtagcatatgtcaaaaattccttcctttctcaggttgaataatattccattgtatggatgtatcacattttgcttatccattcatctattgatggacattttggttgctttcacagtttggctactgtgaataacgttgtcatgaactcaggtgtataaatatctgagaccctgacttcaattattttgggtataaatccagaagtggaattgctggatcatatgctggttctatttttacttttttgtggaactgtcctacttatttttccacagtggctgtatcattttgcattcccatcaacagtggacaggtgttccagtttctccacgtccttgcaaacagttattttctgtttatttgtttgttcgttgatcatagctatcctaatgggtatgaggtgaaatctcattatagttctgattagtgatgttcagcatcttttcatttatttgctggccatttgtagatctttggagaaatgtctattcaagtcctttgcccatttttgaattgggttttatagttgttgagttttaggagttctgtattacacttgatctcatagtcactgatggctcattgcaacatttacataaaaaccagaagaaaagagaagaaggggagatggaaagacctttaatttttaatttttgggggtacatagtagatctacatatttatggggtacatgagatactttgatataggcatgcaatgtgtaataattacatgatagaaaatgtggtattcatctcctcaagcatttatcctttgtgttacaaatgatctaattatacttttttagttatttttaaatgtacagttaaattattattgactgtagttattctgttgtgttatccaacactaggtctttttcattctttctatttttttgtacctattagccatccctactttcctcttaccccctactaccctttccagcctctgataccatccttctactctatctccatgagttcaattgttttgatttttagctcccacaaatcagtgagaacatgtgaagtttgtctttctgtacctgacttatttcacttaacgtaatgacctccagctccatccatccatgttgttgcaaatgacaggatctcgttcttttttatggctaaatagcactccagtgtgtatgtgtaccacattttctttatccattcatctgttcacggacgcttaggttgcttccaaatgttggctgttgtgaataatgctgcaaaaaacataggagtgcaggtatctgttttatatactgacttcctttcttttgtgtatatacccagcagtgggattgctggatcatatggtagctccattcttaatttttgggggaaacttcaaactgttctccatagtggctgtactaatttccattgatgattcttaactggatcaattgtgttgattacaagatggccaatcatatttattagatgacattcttctgccaggaagagcttttccatatcttccatttgtttgtttgttgactctatttggactcataggttttaattgtatttaatgtattatagcccattatggctgttatttcttttgatgctcaagttttaccagagcaatctgcttaacccaacatttccttaactcacttccctacaaaatgtttttcctttgggatacccattggtatcctgttgagagtgttcaatggagtgcaccatggtaatcctgctgtcacaccttgagagtggcacagtcattctctatagcaatccacttgtggctggagtgataaggaaagcctttatgaagaaattgtttgaagtcttctgataagtcaaaatgcaggtggaagaggaatggcatagtcaaaggcacaggaattagaagatataaaatgtatttttagtgcactaagatgaccagcttggcagggaagtagaagggagaaaggctgaaaatgaagattgggaatatattgaaaagggccttgaaagacagcctaaggaacttggatgttaccttgaatatgtgggtggtcatgaaaagcactgagcagggaactgatgtgatgtgataaaagcagaactgcagtacgggagaaatgaacaatgtggtggtaagctaagaggctcttaatgttgtctacaagaaaggtgataaagattgctattttgtgcatttatgattacttctatgatttgtaaccaggatattgaataagctggactcatggggtgtaatcacataagaaaattaatttcatctactttcctttaaactttattcaaaggacaaattctggcagtttcatgaactttttattactcttagtcaataaaaaatcttatggttatttttaagttgccaaacagtctcaattcaggacggcggggtgaggatagcaagaaagctctcctttaattggtactattgtaagatggcttccggctttctgagccttttaggtgtttggagctgacctttcattcttcttgtgatggctttcaaggatcttcaagggccaccacagggaatatttgcatgcatgtctcatgatatgagtgatgcacttttctcaggctgctgttctccatcagcctccagtttggggaagtccactccacacagtctctctttattgaatgctctatcctgtattttgggaaatgatttaaagacctccaagccagctctttttatctcatgaatggcttgcattacatccataggcgacacttccacatcctctgctctaccaagctccgggagtgtataccaaccccagcacagccacctctccaacttaagtttaatgaggagtttaaacttgtcattgtcggtattaatttcttgttaagcctatctcttcccctatgctgcaaaggtggcatgttagatttttatgtagcacaaagcaagctccctcactaagtttgaggtgggaggcaaatattcctccccttctcctttaggaaggtgtgggtagtatagttccctccaacgaaaaccttatcatgaattctttctctttattatcccaataatatacacccttttaagctttggtaatgtgtgaagagtactgaaatcggtttttgtagatttcctttgacaattcacagtgggaactgacatcgcactttctagaccactttttccaccttattgaacctagtgatggatctttggtttgagatttctctttcatttctgacatgtattagtgttgactaatacgcatcacatgccaacactctgctctgagcacaggccaacagaatggccttccacagtcacatgcaggatctgagattgtatactcaaagaggcaaagtccgagatctgcatgccatgctctcagatgcccctgaaattcacaggtgtgatctagcagttgaaatgagatgacaggttttatttgaaggggatttggccttggtcgagccagcgtggaaggtgcaatgcctgcatgttctgtggtgggtcccaaggtgagcattgaggtacaattttagaatttaaggatttgagtatatacattgtctggctgtagtttataggttcattcttatggagttgccataaaaataccttagttttcatgatctatttagcacattgtattaggccattcatgcattgctataaagaaacacctgaggctgggtaattataagaaaagagatttagttggctcacagttctgcaggctgtacaagcatgacaccagcatgtgcttggcttctggggaggcctccaggagtttttactcatggcagaaggcaaagtgggagcaggcacatcacatggccagtaagagcaagagagagagagaagggggtgatgccacatacttttaaatatctcgtgacaactaattcacaactaaggggatggcactaagccattcatgagaaatctgcccccatgatcatattgcttcccactaggccccaccttcaacactggggattacaattaaacatgagatttagaggggcagagtgcaaactatatcacacttcctactaactctacaaattattgagtgagacactgaaggaagaaattaattgcagaaagatgactaagtatacctatgatatatttttctttcatttattttaataggtggtggagaaaaatgcagatccagagacaacccttttggcctacctgagaagaaagtgtatcctgactttggagtgggagtgacggagaaggggggcaatggtgactgattctgtaggccttacattgtttcctagtgactggtggggtggccttgaggtgtggaggaaagagcactggattaggacttgggacacttgcattttagttgcacctgtaatctgaaattctcagctactgaatgagaggattgtttgtggtgatcttcgaggtgtctccccgctaagactctagtaatgccaaggtttaatccagggcctgtagaaaggtcctcattagccttggtgagcttcatggtcattcctctgtgggaagaggggggccttccttaggtgacatttgcttaatacataaatggagttcctggggaatgtttccagctctgcctaggtggagggaagcaagggaacatgtgtctattgggaaaaggatgcgtgaggtaagaatcctatacaggtagcattcattgtagaaacagcaaacagggagccctggagggtggggggactccttagaaagactggggtgctgctcttcactcagtgtgggtggggcttctggaaaagcccctttacctttagttctgagtatcggaagtattctgggaccctggaactaaaacgataataataattggagttaatatttaatgagcacttcctatatacaggcaggattcaaagtgatttgtgcagctcacctcaatctccacaggaatcccatggaagcgtaacactcccttttcacagaggaggaactgaggccttgatatgtgaagtgacctgccttttgctagtgtgttctggggaaatagtgatggagattgcaccaaagcctagagctgtgttcccaacctctcacccgcactggcaactctccagggggcctgtgagcaggactgtgctcatcctgacctctgtcaacaaaaccctccttctcctagcgggggtgtctagtgggtctttccagtgacatatttggggacagcaaagattattataacacgcacagaagtgagactggagcccatgctttcactctggtttgctcccctcctatcagacagtacagtggttggctttggtgtactttgttctggattttttaccttaagtgggtgtatctggaggttcacagaagataaacctggtggtgctggggagtgaaagtgtcactagatcggatcaggtgattgcatggaactccatttacagcccatctacctgggaggtctgggaagagaagctcattctcagagattattgagatcaattttagctttctaataataaaacaaattttaattttttaatcatttccattgtagttttgaaggcccagaagcctcagaaattccttatcagcaattggctgtgtcacatcttaaatgaaatgcaaaactgaaagttggcacataagtaggatgaccttgcagcttatggctatctgtgctgagagtcaatttatgggagttccagatgggggagaaaactccagccttgacttttacactagcttccagaaaggtctccaggctggaagaagaccttgctagactttgtactgccaccattcatcttcaggtgccaccattcgtcttcaggtgcccccaaagttgtttgacctgctgagagctgcatatatcctgactaggacacagaaaaccttcccaggtgtgctggggaggaaggagcgtgatgaagggagctcacccagtctggagtccgatggctgtgatggggccacaccggcagggtaacgtgttcctgtgtcatcttgtgtggcccacgttacctcttgggtctcagtttgttgttttttttaaatctgtgaggaagtttctttcctacctgctaaccaaatcattctggaggtcaaacaggtatctaggtgaaagtgcttttcaaaaataagatgttatatcattgtttttaaattttgtcttgaaaatgagagaagtgaccaggcgattatatcctcagttccctccatcttcagtaggtagtgggtggcttagaggcctgcaggtctgagggagaagcagccatgtgactcagggaaagtgacctccacaatagctgttctctagaatagcttaccagttaggcacttttaaaactcaaagtagcagatggtctttctgcaagtatatgtaagagtgaggcccaatattggacccagacctaaaaaaaccactctattttaatttccattcacatatcttttggaccaaatattcaaaagcctctggtctagccctaattttgattcttggggacttatcagcccaaaaagttcagagtgaaaagcaaggactcagaggtcccagaacagatcaatgtctcattctggcccaagtggcttcatggtgtgcccagctgggagtgatgtgagaactgccccgtatgctcaggtgtatgatttacctgctcacacatagaagacctcaacccaagagaaggctctaggactgaccagtcctagaactcatgtcccaagagatgctacttgggtgctgtccatggggttctgatgcctttgctcttctgtgcagtggagatggccgtgttccctgcctccctagggtgcagtgcagcccctctgaataaggaggccagtgggcctgctgccttccttggttgaggggcctgagcacgtgtgggaatgaatggctctgcccacagctctgcccaggcatttcctagcaacccagcagactcctctccctgagttcattctccttaactcttgaccacccagtggggctgagtggaaccaagctcggctgtggagaggggggctgcggggcttgcacagtgatgctctccaagtatgatcgtctgcagaacaagatcgtgtatcctttgcctgctggctgaccacccacatgggggctgcgatgcaggaccagcttgtgagggagtgcatgagtgtatgtgtgtatgtgcatgtgcgcatgtgtgagcccctgtttttatggagaatctgtgcacaggaaaactgtagaatgatctcttagcaagtatcttctaggcttataatattgaccaggctccgaactgctgataaaatgacttttctaaaatccacatttatatcacattccataaagcccttcaatagctcttcattgcccccaggacaaaatctctttgcttagcgggcaaggccctgcacagtatgccccaggtttcttctccagttttctttctcaccatgggcccagcactccattcaagcagagataattgtgaaataccaatgacacaggcccactaattgcttggtctttgtttaagctgggaagttctcctaggatttctctatctgactaactcttactagctctttacaactgggcaaaaatatcaccccctccaggaagccatcctggatctctcaagactgggttaggtgccccttgactgtgctggtcccatagtgccctgtgcacagcattcccaatgtaaactcaggcatgatacaatttttacagtttatttgagaaaacaacagttcatgagctcaagcagctccacattagaagcagttcaggagctccaccagggaaatgcaagcaggaggtttttgtaggatggacacaggcaaaaagcaaagaaattatttgattggttacaattatacaattgccttacttggtctatcccactggaaagtccctagtactatagttataagtttgttgactacttctgattggttgatcgtaaggcttttttttaatataaagatattcgtaagaagtagctcaagttaagactctgctgcaaatcaagcacagtttaggtcacttgtgaggcctaactggtttcctctgctgagtcttcaggcctggtctcctttttaatttactttaacaagatttaaattgcaccttcatatggactttggttttctcagttgactgcagtctgttgaagggcagagctttcatcttattcatctttattttcccagccctcagcagagtgcctgacttatgtctggcactcaataactgatgctgaatgtaagtgaatgagtgatgaataacagaagaatgcaaggaagttctagctaagggcttgctgtctcatgttgcctgggggtttcctccccctttccccacagagaaatgaaagcaatagaggcgaggaaatgcagggaagcaaagtgattggaagctaaggaacagccctgcaggcaggagcatgctttaaggctgcagcctgccagagaaatttcctcttgactcagcctcctgggtcaggctgagtaagaggtgactgaaagtgaccctggctccgttgctcctgctcagaggctgaggatgctctgatgcctgatcccacagtactctggaggggctcagctttacctataaggagagcttcctttgttcctttcccagagtcttcagtggagaggtgaggacactggtagaagagacaagaacataaatgggagcggagagacgtaacataaagcctcacaaagtcctccagagctcacagtcaggcccattctgatgatctgagtggacaataaatggaatgaccttaatctggggttctagccacttttctgccaatgcctgcctggcccccatctgctccttgcaccatgttgcagtgacaactgtggaaggaataggaagcaccaagacgaggctgcatcctgtgcaggtaaacagaggaaagggagctgtgctcttgagatcaggctctttcctctgcagggaggctcatgccaaagttgcagacttgctttgggttgttgggaagagtttcactgcttaatctaagaaggcatttcaccctggccacctccccatctcctctgggtaccgtgaggtgaaaagaagaaacacccaagcctgaagctccttcctgtgagctggttggttataacattgcatgatctgaagggacccaagaggccaactgaatctggtggctagaccagtccagaacttggaggaagggatgcagaagagcttcctcactttgttctggagtgtgtttaggaaactcccagccttcccagatttgaattttaattatctgtgccccttgtttataagcccacttactccctgcaatggtaattctgcaccccttacctcccacccgcagatagtacaaatgtaaactgctaggcctcctctttcattagactggaggttagcaagtgctaaataagatgacagagatttagtgcctgagcaaggctttctccttgatggtatcagaccacctgaaaatgcattgaaatccgaaacatttctggggcaaatgggcagtaataatcttagactggtttggggagacaatcaggatgctgttaggctgagtggaaagaaaggggagtagagccctgttggaaggaacggccattgtccccaaagcattttgtttgaggaaacttggggtctttgggagatcgagactatcctggctaacatggtgaaaccccatctctactaaaaataccaaaaaattagccaggcggagtggcgggcgcctgtagtcccagatactcaggaggctgaggcaggagaatggcgtgaacctgggagatggagcttgcagtgagccgagatcgtgccactggactccagcctgggcgacagagtgagactccatctcaaaaaaaaaaaaaaaaaaaagaagttaccagagcttggcccaggtcttaatcctcccttgattatttgaggagacatgtcattcaggcgatggagctggagcctcctgcggagaggctggtactattccttggagtggcatcttgggccagtcatgtgaggccagccaggtcggggtgggattgcttcccatctatagatttgtttcttaggcaggaagaggggaaattactaaacttgtccaacctgaaggagacgtgagagggttaattaaggcttgttattttctttgaaacaaacagatgaaagaaagaataaacagtttaaacataggagtcaagtattagtctattctcacactgctgataaagacataaccaagactgggtaatttacaaagaaagagaagtttaatggattcagttccacatggctggggaggcctcacaatcatggcagaaggtgaaaagcacatcttacatggtggcagacaagagaaatgagagctaagtgaaaacagaaacccttcagatttcgtcagatttattcactaccatgagaacaggatgggggaaccacccccatgattcagttatctcccacagggtccctctcgcaacacatgggaattatgggggctacaattcaagatgagattggggtgaggacacagccaaaccaaattaggttgtatctgtataatcaacctaaaatcctccctatccaatggtacaggttcttattcctaatcaacttattttggctacaaataggcttcagagccagattccacttcttgtccatttatttgttttctcttggtttctgtgacactgtggttctggcttttccttctctccccacacttgctcctcctctgctcagtctctaattgctgcagtgccctgagccctgacctgggcccctccccttcttcatccctatactctcatttacaacttcagctttgacctcccctcagaagttacatttccaattgcctatttgacatctcaatacggatgtctaatctcaaacttaagaaaaccctactcttttcctaatggcttccttccccagtcttctcccatctgagtcaatggcaaagccatccacccaattgctcaagacaaaaatctagacatcacctttgactgtttctgtcatatgccacacccaatccattcagaagctgccttctggttatgatagtcactgttctttgaagggaccttcctagtcatagctaacactggctgactgcctgctatatgcaggtacagtgttaagggttttacatgaatatctcagttaatcctcatcgcagctctagaggtaactacaaggatggtcctcatcatacagatgaagaacctagactcagagggcttggaaactggccaaggtcacagttggtaaatggtggcactggggcttgactctgtgcagtgattactacccccactatctctcaacatgtgctggctccagtcgagtcctttggggagcctgggagggttctgccatcccaggtgtctatgtcatctgtatcttttaggagagaattgccaaaagccacggctcccagtgcgggttctgcacccctggcatcgtcatgagtatgtacacactgctccggaatcagcccgagcccaccatggaggagattgagaatgccttccaaggtatgggccttcaggcacagcctaggaggtggaatggcaaacgaggccctgcagaggtcagggcaagtgccagggtgagaagcatgagggactaccctttggaggaagggctcaggccccccaacaaggtggtgactaagatctgctagaaagccaaccctctcttacttgggagaactcctggttggtgtggagacctcctggggtctcctgcctttgtgtccatatttctgaggtgtctgtctgccttccaccagtattggagtggggttggcagtgcatggctggtgcaggaagacgtcatttagaaaactccacgggggctagtgatatgatttccttccctctcagttttcaccagaacaggattctgcagggccaagaccaccaatgtgtctgcttctctgaattgaatagagaagcctccccatctattcaccatttccatcatttattcagttattttagtcacatcctatgccacagggataaaagacacaccctctactctcaaaaagttcatactctgaagaggagagtttgtaatgtagtctgataggctaactgatgaagctgtgatcagccactgtgggagcacttaggagaggtacctgactcagtctgggcattctaggaaggcttccattctaggaaagacttcctgaaatcatgataacttttctgaagtcaggatatgagaaggccaagaggccttccagacagaggtaacagaatatgcagcctggtaaagagagcacccaacttagttgcaagcaaagtcacaaactcctcacccatcccagggccctgcactgacattgcagttttttccttggcccacaggaaatctgtgccgctgcacaggctacagacccatcctccagggcttccggacctttgccagggtaagtggggaccccagaacatcagtgtctctgcttctaaaatggcaacactcagaaatcactgtggggagggagaccaagggcctctggtctacaaacaatgatgataagatgaccagttagtgagtcttccctctggggacatctggcatggggagtagagtctgccattcttattcagggaagggttcttatgaatagcatgggactttctgccagtgatggtctggagaccggcttcctggaagaggctcaccgcctgagagtgcctttgagcttggcagtgcctttgggcttggcagctttcccttggagactggcaggctcagcccatggcacaggtgctgaatgtagcatttaataacaacgtggccttgctcagcacttgcacccctcctccttccctctcctggctgctttctgtggctggcactctgttgggcttatgaaactggtaattagatctccagacagtttgtgaacccctgcaatgttcatataactctgagtgagaagtcacagccggatgtgtgggcattgattatataaacacatacacacacctgccatctggcaaggaaggcagagagctggcatctcctttcttggcttaatctccccccaggttttacttgacttctcaattgggccacgttaatcctgactccaaatcaaaccatctcatttcagtttgattctgggtccaggggagttacagactcctgtgctttccactcacctttagggttggagcatcatgaaaaagagtgctgtgtctgtataatcgtataatccaagaaggggtgggtaaacaaggagacaggaccttataaggatggaaaacaccacactctgattaatgctgctgctgatgaagaattaacaagtgtggaattaaatgaaaatgcctaaaagcacaaattcagattctgcagacctgggttcaatttgaggcacttaattgatgcacgaccttgggcaagtctccttgtttctattaggttggtgcaaaagtaactgtggttttgccattaaaagtatgggaaaaactgtaattatttttgcaccaacctaataaaatggtgattttattaggtgatttcattagaatatctaccttactgtgtttataaaaattaagtacttcttgaaaagcttttagaacaatgcctggcacatttctgttattcaataaatgggaactataaattatgaagctggtatatttcttccagtagcctctatataatcttcccaagttactcactgtttcctgagacccagagattgttaatcttgcctctatttacttcataagaacctgctcaaagtgatataatgtaatcagtcctcttaatttttcaaaagagaaggatttttgagtatagcagttgccttccctcctggaagttgaaatcgctcttgatttcaccatgcagaacttacctccctcaattcagctaccctgcagtgcagagcagagcaagactgagagctctgagcagccaaaatgcctgacacacaaaaggtgctcagaagctaatgcatgttactcatcggaggtctccgagatgtttcttcagagcctttttacttttattttacacacagctctaacaaactcggttactagctttcctgtccacagcacacactagaaagcgtgtggagggtcaactagaggtgggcacactgctcacggcgagaagtgaccacagctatctggtgacaaggaaagaaatggaggaaccattaaacaggtacaagcaagacttccagtttctggtctggcatgtaagcagcttaggagtctttatttcatctaaacaagaagaaaaaggctgaacaaactgaagaatcaacagttgtttttagatccatcagaggaatgaggtcacaggacaactcattgtccaaaaaattggagagacagacaggcagacacggcatatcagagcagaaacctctgcaggaacctttgccaggataggaagatgtaaggactaactgatgaattgctggagtttatacatggatataaactccagggggatgcagtcatggagcgggtgctctacacctttgtgagttttacctccaggatctccaccaggtcctcacagtaaatgttggagaaaaatctccttgtgtttctggtagggagtgggaaaaatgaaccattttggaatatgccagagcattctgttcttcttaaaaaggcctgtcctcagaagaaattattttaccgcagcctaaacttctgaggttatattagatcctcgcctaccttggggaggggaaatacgcaaccccaggacctttagtctttcacctgtgggaagggaaatacacaagcccagccccctccagccatcctgtcccaccaaacggagaaaaaaaaccctgagaagcgcttgtgaagttcacaggtgagcggcacaagctcaccaaaaaactgagactaatcctaggactataggatacttctccttcatatcttaccaatacattaactaaaggcctatttatatgaggtcttttcagccagtacatcatgtccacctttcaaccaaaaattataaggcatagtcaaaggcaaacacaatttgaagagactgaacaaacagcagaaccagagtcaaataggacaggaatattggaattaacaaatgaggaattttataaagctatgattagtatgctaagagatttaatggaaaaaatagataacatacaaaaacagatggataatgtaagcagagaacggaaattctaagaattaaaaagaaatgctagagattgaaaacgcttataacaaattgaagactaccttcggtgggctcattagtagactggacatggctgagaaaagaatctctgagcttgaggatatgacaatgcaaactttcaaaaccaaaaaacagagaaaaggaagtgaatgaaaacaaacagaaacaaataagaactgtgtgataactacaaaagatgtaacatatgtgtaacgagaatatcagcagtaaaagaagaaaaagagaaaggaaaagaagcaatatttgaaacaaaaatgactgagaatttcccaaaattaatgtcagattttaaaccacagagtcaggaaattcagagaacacaaaccaggataagtaccaaaacccaaaaccaaacaaaaactataccaaggcatgtgatattcaaacttcaaaaaatcaaaggtaaagaaaaatgacttgaaagaagccaaaggaaaacacccctcacctatagaagagcaaatataaaattatatctgacttttcccagaaaccatgcaagcaagaggcatggagagaaatacttaaagtattgaaagaaaaaaccaaagcacctaaaattctgtaacctccgtaagtatccttcaaaagcgagtgagaaatattttctcaaacaaaacttgagataatttgttgccagtagacccatcttgtaagaaatattaaaagaagttctttaaagagaaggaaaatgatgtaggccagaaactcagatctacataaaggaagagcatgagagaatgaacaagtaaaggtaaaataaaaatttttatttttcttaatcttaattggtctaacagatagcaatttgttcaagacaatcatagcaaccatgcatttgtttatgtgtgcatatgtatatatatatgtgcttgtgtataagtaaaatgaatgacaacaatgataaaataaacaagagggaaaaattagaaatattttgttattataagatacaatcactacctgtaaagcggtgtagtgttatttaaaagtggacttggattagttaaaaatgtatactgcaaactctagggcaactaccagaaaaaaaaaaaaagaaaaaaggaagtataattgatactttatgaaaggaaaggagatgaaattatatataatgctcagttcaaaccacaaaagtcagaaaatgagtgaaagacaaaactagaaacaaagaataaggacgacaaatagaaaacaataacaaatatggtagatactaatccaactatatcaatgattaccttaaatgtcagtgatctaaatacaccatttaaaagacagagattgttagagtagatgaaaataacaagacccaactatatgttgtctacaagaaacctactttaaatgaaaagacacagatagattaaaactaagtagataaagaaaggtggaccatgatatactaatcgaaagaaagtggaagtagcatattaattttggacagagcaaacagttatcagagaaaaagaggggcattaaaaataataaaggaatcaatactccaagaagatataacagtccttaatgtgtgtgcacctaacaacagagcttcaaaatacatgaggcaaatctggtggaactgcagagacaaatagatgaatccattattatatgtgaagacttcaacacccttatatcagaaatggacagcaccaggccaaaaatcagtaaggacatagttggactcaacggcaccatcaattaattggatataattgacatttatagactactttagccaacaacagcagaaaagacatttttctcaagctcacgtggagcattcacctagatggaccacattctgtgctatgaaacacacatttacaaatttaaattgcatgaatagagatcatacaatgtctgctttcaaaccaaaatgaaattaaactagatatcgataacagaaagatagctggaaaacccccaaatgcttgaagattaaacaacacaattcaaaagaacacatggtcaaaaagaaaatctcaagggaaattttaaaatattttgaattaaatgaaattgaaaatacagcttatcaaaatttgcaagatgcagtgaaaacaatgcttagagggaaatttatagtattgaatgcatatattagaaaggaagattaaaaatcaataatccaccaggcacgacggctcatgcctgtaatcccagcactttgggaggatgaggcgggcggatcacgaggtcaggagttcaagaccagcctgaccaacatggtgaaaccctgtttctactaaaaatacaaaaattagccaggcatggtggcacacacctgtaatcccagctactcaggaggctgaagcaggagaatcacttgaacccaggaagtggaggttgcagtgagccaagattgcgccattgcactccagcctgggtgacagagtgagactccatttcaaataaataaataaataaataaataataaaaataaaatacaaataaaatcagcaatctgggctcccactttagaaaactagaaaaaaaaaggcaaattaaattcaaagtaagctgaagaaaaataaaatttggagcaaaaatcaatttcattgaaaacagacaatcaatagagaaaatcaacataactgaaatttgggtccttaaaaagatcaatgaaattaataagcctctggccaggctaactaagaaaaaaaagagaagatccaagttactaatatcaaaataaaaagatctcactatagagctcatgtacattaaatggataatgaaggaatattaagaacaactctatgctcatatacttgataacctagatgaaataaaacaattccttgaaagatgtaagctaccaaaactcatacaaaaagtagataatctgaataggcctataactattgaagaaattgaatcattaattaataacctttaacacagaaagcaccaggcccagatagattcactggtaaatcctatcaaacatttaaaaaagaaattatgtcaattctccacaatctcttccagaagatagaatcagaggcaatacaaaagttaatcactttcatatgtaacagcaatgtataagtggaacttgaaattcaaaacacattatcatttacgttttacattaatactctccaacaaatgtaatacttaggtatgaatctaaaaagtgtgtacaagatttatgtgagaaaaactacaaaactctgatgaacaaaatcaaagaactaaacaaatggagagatatttctttttcatgaataaggagactgaatatgtcaagatgtcagttgttcccaactgtagactcaatgcaatcccaatcaaaatcccagtaaattattttgtggctatcaacaaattgatcctaaggtttatatggagaagaacaagatccagaatagccaactcaatattgaaggagaatagagaacaaagtcagaggactgacactacccaatttcaagatttactacaaagctgcagttatcaagacagtgtagtatggacactgtccatactacaaaaagaatggacaaaagaatggacaaagagatcaatggaacagaatagagagcccagaaatagaccaacataaatacagtcaactgatgtttcacaaagaagaaaaggcgatacgatggagcaaagattgtctttttaataaatggtgctgcaacagttggacattcacatacaaaaaaaaatgaatctaaacacagaccttatacacttctcaaaattaactcaaaatgaatcataaacctaagtgtaaagcaataaaactcctagaagatgacataggagaaaacctagatgaccttggaatgatgatgactttttagatgcaacaccaaaggcatgatccataaaagaaataattataagctggacttcattaaaatttaataacgatgaatgaggagacaagcggtacactgggagaaaaatatttgcaaaagacacatcttgtaaagtacttatccaaatatacaaagaacttataaaactcagcaataagacaatgaacaactaaattaaaaaatgggtaaaagacctgcacagctacctcaccaaagaaggtatatggatggccagtaagcatataagttcgacatcatatgccattagggaattgcaaattgaaagaacagtgaaataccaccacaggtctattagaatggtcaaatccaaaacactgacaacatcagattctggtgaggatgtggggcaacaggaactctcattcattgctggtgagaatgcaaactgcacaaccactgtggaagacaatttggcagtttcttacaaaactgaacatgtcttatcatgcacctcagtgatcacacaccttggaactgactcaaaggaatttaaaacttattgccacatgaaaacctgcacaggcatgcttacaacaaccttactcataattgccaaaacttagatgcaaccaagatgtccttcagtaggtgaataaactgtagaacatccaaataatggaatgttaattcagtgccaaaatgaaaggagctatcaagctatgaaaagacatagacaaaccttaattgctactactaagtgaaagaaatcaatctttagaagctacatattgtatgatatctattctatgaaattctagaaaagacaaaaccatggaggcagtaaaaagactagtggttgccagtgattaggggaaacagagggatgaatagtcagggtacagaggttttttagggcagtataactctttctgtaagatatcataagggtcaacacctgtaatcatgcatttgtcaaaaccaatggaatgtacaacaccaagagtgaaccttcatgtaaattaatggactttgggtgataatgatgtgtcaatgtgggtccatcagttgtaacaaatatgccacttttgaggatactgaagtgagaaaggttgtgcgtgtatgttgggataggggtatttgggaattctctacatcttccactcagttttttctgtgaaccttaaaccactctaaaaaaaaaacttttttttttaaaggcaccagtgcagaagccttagcctatttagtaaagtaggaatctcaggagggtggggatgagaaaatgtagtcttatccaacctcaatagaccttgagggcagtgatgcattactgtacatcacagtaatttatgtcattaacacaccaatgaattatcaagaaaatagatctaagtaataatgcactggcactttgtctggaaaaggcgcttctgtgaaatatgtccttcctcagagggccaggtgagtgggatgcaggactttcacttcaggtaggccagggcatttccctacatcactccttctccaagaagaaaggtgagaaaaggcactaagaaacctctctttcttcctttctttctttctaaatatagaggggaagtgcaaggtggctggctggggggaggctcagaatggacggagcaggtctggtgtccagtgctggagctgtgaatgcaggaagagtctgcacacggagcagcgtgtccaggtggtggggtgggtggggtgtttctctcctgatgccactgcctgcctgcttacaacttctccaaggtcacacctccagcatcccatgagtgctttcttgcagaagacaacgagttgtgaaacagactctggaaaatgccagcatggcatggtgtctcctgtggctgacttgcagaaggactgctcccagcctgctttttcaggaattgactacccgataaaagtgcctgaggcagaacctatgagtttcccctactttttcatctcagcacaactccaggactctgtcttctgaagccctgagctgcagatcagtcagctgagtgcgcccctctgccaccctgcagtgcctcggtgctgggtggtagtgattctagactctgtgtgtgctctgccactggtatcctaagaggtcctgagcaagtcttttcccagagatgctgggtggtctgacctgcatacttctccagaacaggcccacagtgaggagtgaagggtgtttacctttctgggaagaagtgcctaataagcagtagggaggccattgggcacactggctggacacagtgctgggctcagcacccgtcttggacatcctttggcagggtcctgcagagagcccagcctgggagacaccagccacgcccctagaaccttgctgatctggaagtcaagcctcttgagtcttgagagagccaatgggaatatccctcatagacgcttcctgaggaagcagcactgaagaagagaaatgggcataagggataggttttgataaagtaggacctagcttttatggagaaaagttcctgaaattcagtggccttgttgctcctatttgtttatccatgagttgtttaatctacgttagccaagcaagagggaatgtagcggaggcctccactctgggcaacaggcaggcaggatgcccctgctgttgatgctgggacaggcgtcagagctggagtgttctggatattagtaatctagattccttagcttctgcaaatactctttgtaaatacccttgtgcagatgttcatcatctctctaggatggtggatgctgtggaggagatgggaataatccaaattgctgcatgaaccagaagaaagaccactcagtaagtgagttcttctccccctggaagcctgggggtaatctgagacttagaggagtcagtgcacgagctccatgtccctggcggtgggggctggaagaggaacctacggtgagtcggtgatggcagactgtggggatggtgatggtgcacgttagccccgggtcctgtaactgagactctctcattcctgcctcccagggattacctgctcctcttgcagcctgcagggggctgggaatgagcttgctggaaggaaaggctgaattggtcctcttgggtgtttgaaaggaggagatactgcatcacctacctgttctctttccccataggtcagcctctcgccatctttattcaaaccagaggagttcacgcccctggatccaacccaggagcccatttttcccccagagttgctggtaggtgatgcctcagggacagatccagccgggtctgtactgtgagtccctggaaacctgctttattttcatacccactgggaactttgtgtctttcttgtcctttatgtttcagcttaaatttcccttccattttctgacactcccctaggttgagtctctgctatgtgtacccacagcccctgcacttcagttcatgtgtgtcacagttacagttattaatttgatgtctgtgttccttgctaaatgacaagccccatgaggatagggactggctctctaggcacactgtgtagtgtagcaggttcactataaccatttgttgatctcctgcctatatctaatacataagcactccaagaaacgtattccctctataagcaatccatttatttaaaacaaaacaaaacaaaacaaaaaacgagagctctcttcaaaggacatgatctgcagctcacagctctgtcttaaggaaaccccctcacacccactttatcaccatcactctaatatttatggcaacgaaagaatcaagccactcggagtttttgaagtgcacacttctcagagttttgagctatgaaaactaccttgtagcttcaacaggccagagagaatggctttgcagcaggccaccagcagatctttctctggactgtgaaactttgaaaatactggcagttcgatttgagggcttcgttctcagcccagtgtcttcctgcaagcactaaagccagccttcctaggcatccccttataacaaactccttccttccttccttccttccttccttccttccttccttccttccttccttcctttcttccctccctccctccctccctctctccctccctccctccctctctccctccctccctcccttctcatgccttgtccatggtgccccctggagatattatggggcactgcaaggaatctggctttcagccaggtctgttggggacccctcatggcaggtgaccttcagcaggtcccataacctccttgaccctcaggatcttctttcactggtgaaaatgaggctcataatgcctccttcatggaagtcttgtaaagactagtcccaatggagttcctgcctctgaacatattacttccccttgcttcttcctgttagatattttcattctgaatgtcagtaaaacatcccattgaggacttaaagcaatttataagaggaatgacagtaggaagagggagacattgtagtaagaaaactgcatttcccagaggctgaaagacactcctcggaagcagctgcgatttgaaggggagcgtgtgacgtggatacaggcctcaaccctcaaggagctgctggacctcaaggctcagcaccctgacgccaagctggtcgtggggaacacggagattggtaagggccagggaggctgccctggggctgccagggggtctgggggtgtttctaggtcctcttgcccatcctcacctccccctgactttacttgcctctgcaccctatccctctgccccactgactacctggagcccactggaaatccctggacactccagttcctgcaaaggcccctagctgtggagtcccacaggcctggctcagccccttgctggatgtgtgacctcagaaaagttgcttgtcctcatctaccctcattttctgttttttatatggggatactaacaactccttgaaggattgttttgggtgtgaaatgagatccttgcatgtagttgacttctctccacaaacatttacagagcaccgagcatgagccaggtactctggaaggggcttgggatacaaagctcaattggacatggttccttgccttcaagggccttctagtctaagggaggaggaactaatttaagcaaagtgtctaggccagtggctgatggacactagaggttccataaatgtcgcttttgaccccaccaccccactttttctgggggaggatgtgataaggtaaagaagaggagagctccaggtgtgactgtgtttgtgttgcctatgaccatggccacctgccctgtcagtgctaccacctaggacgtgaggcctggaatagttgaacaggctgggagactcagaatatgcacctctcagccccagtgctgtaagagatggggagaggttcccaggtccctgctaatctctgtcgatttctaaacctggcatggcctatttctactttgctggatcccaagcctagtgaggatctaaggaggagaccgtgggacagctgaggacctctgctgtggaggggcatgctctccttctaagcagtcaggtcctggctctggttcctgaaggagctgccaagccctgagttctggttggctcctcagccacagagaggagagcctggcttgggtcagtgctgatggagtcagaatggacgcctccctcgtgcgcacatatacataagatagagtccccaactgtggttttgtgacacactgaggcagcagggtgggctgagacacacctcaagaaatgtgatcttgatggccatggctcaacaggactcatggctcatgtagctcaggtgtagcttgatttaaggtggctgggggaatcttaggaggtccttctttttttttcctcaaagacagggtctcactctgttgcccaggctgaaatacagtggtgcaatgtcaccatagctcacctcaaactcatgggctcatgtggtcccccaccccagcctcaaggggatcatttcacacttgcatgctccctgccatgagccatccaatgtcctgggcccagttggattccaagagaaatgaataacaaagattacctaagttccacaattagaaatgatttctcaagaccatttccttctgcgccactcccttctctcttatacataccccaatagcatagtcacctccagtcaacgcagagtgttgggggaaggcagacaaagagaaaaccttagtaacgtagaacttgctgcttcaatctgtgttctttgtcctggaagctgatattcaaaaggggctggtattcaggtgcttctaggatttctgaagggagctttgaagattctggaatctggggatccacaaagcctagggcccccttcagaaatcctaaacagcacctggggggaagggggggcaaagaggtcacaggtatttcttttttaaccttttccatctatacactgttggcagggctgggagaggtgctgagccaactgggtctaaggaacagaggcacacttgtttaacagaggcttctgactcatttagtcaggaaggccagaaaccacacacagatgtgtggccccgcaaagccagacaacttgaagaactcaggcagatagtgggaactggaggacagatttgaagttttccctcccattttccttttttgttttaatgttaaactttcatgtttttattacgattataccccaacacgaataatcattccagtactgaaatttggttattcttagctgtgtgatttgggcaagaaacttaacttttctgtgcctcagcttcttcatctataaacagaaaaatcatagtacctcatagggttgttgtgagcattaattaagttaaaatagggaaagtgcttagaagggttcctgacataccttaatttcctgtgttttctaatgtacacaaacacacacacacacacacacacacacacacacacactagagtgaattcaaactcatttcatttgaattcaggtaggttttttgagagggaaagaaagacctagttttgggctgacagtccaaagactgcatctaatcccacccctggtctactgagtttcctattcccagacacctacagtcaccagagagcatattatgattgtggatatgtcatatatcctgtgtgtaatggtgagaaaagctgctaaggcctgccctggtgatacaacaatgggcctacaacttcagcttcttgtttctctctaggcattgagatgaagttcaagaatatgctgtttcctatgattgtctgcccagcctggatccctgagctgaattcggtagaacatggacccgacggtaagaggaggttcactccaagcttatggaggggctgtgaggctggggataggttacaaggtgggtggcaggtatcagggtggtctccccactggctctggctgccccctgcttctcctggggctcaccttgcacagggaaaggcgggaggagaccccattcattgctgaaacatccatggtagggtgccagtgggcaagaggctgcgggatcttcaatcatttttctcaattctggctacacattagaatcactcatgcgggggtggggaggtggggattgtgcctgagtgcttttaaaaagaatgctaggacttcaccttcagagattctgattttaatgagttgagaactactggtttagaggaaagagcattggttgtaggatcaagaggcccaagcttgagtactggctcttttattagctgtgtggcattggttaggtcactcaaccactctgggccctcatttcctcatccatggaaagctgtgagtcctgattcagccttgctccagttgctggaaagcttcagaggagctgtgcatgaaagcctcttctgaactgtgggaagaattgctgtgattccgaacgtgcgttcccaggtatctcctttggagctgcttgccccctgagcattgtggaaaaaaccctggtggatgctgttgctaagcttcctgcccaaaagacagaggtgttcagaggggtcctggagcagctgcgctggtttgctgggaagcaagtcaagtctgtggcggtgagtcgctctcagaagcaagcgaggcccgtaggatggagctttcatctgaaagcagggcagccagactctcactgtgtcagtgcagtgggcctcaaactttagcgcttagagttgcctgggaggagcagtgcttgttaaagtgcagattcctgggccccaccctcagagattctgatacatgggtctgggatgtagcccagctcttcaggtgcttctgatgaaggtggtctgtggggcaccctgggaaatgcactccacgactcataggcttcattctgtggttgcagagatgaggcttcctgcctccctcccccttcctgagcttggctgagccaaacagtaggtgaacaagagatatggatatgctagggagaagtcagaattgagaggggaatttcctttcagagctatagggagaaccagagacagagccctcccacagagggaggaatacacctgttgattattcagtcagccttcagacaagtccccacattcccagtttgcaaatggtggcattcgaacaccctatgccaatggaagctcttcacaacagtttatggtgtcggaagaaaagctatctcttccatttttaaattgtttttttaaattaaaacaaaacagcctactcctattcctcccagcccccaccccaagtacctgtcacttctgattatagataaggaaacataaactgtagataaggaaacataaactcaaaaactctgctgagttccctagttctgctaaaatctatttccaggaagaattgtattttcatcgtgcaactgattttcttcctgttttctaataacgagaaccctgtatctgattcttcttacattttttttgccaagttaccaggaagttcagatccaaatagagcaaattgttcaaccttaagtcttcctcctctctttccttgatctttatcttctattttactaagtctatatggcctctgtgataaatcacctcaatcctttcgggaaaaggttgggtgaaatcatcaatcaaacacacacacttaaaggggcttgcctgctaaaattttagagtggggatccaatctcaagtattctgatttcaaattttgcgcatttttcagaatgccgaagttctcactatgctggtattgtgactacagtaggcacaggacctcaggggtttgcctgcagcaggtgtcacctggctggtatgttcaggagcctgctacgtgagcaggggtttcctggggtgggctctcactgcatgctctctgcttgttttagtccgttggagggaacatcatcactgccagccccatctccgacctcaaccccgtgttcatggccagtggggccaagctgacacttgtgtccagaggtgagctgcctgaagcagaggtccaggaagaaggacttgggggagaaaagtctcaccagataggtcattttctggagaaacagagctcaaagttcagtgacccagctttccctgtgatttggatccagggaagcatggagaatgtacctggtttggtggacgggaaagctggatattttctctgtagagaattcacagcaggaactgctacctgcatgccttacctagtggggtactggggctgggatgcactggctcaccacagcctgctgttggatcttcaggaattctgtgaaccatttgtgaaacacagttattaataaaaatcaggccaagtgcggtagtgcggtggcttatgcctgtaatcccagcactttgagaggctgaggcaggaggattacttgaggtcaggagttcaagaccagcctggccaacatggtgaaaccccatgtctactaaaaacactaaaattagctgggtgtggtggtgtgtgcctttagtcccagttccttgagaggctgagtcaagagaatcacttgaacctgggaagcggaggttgcagtgagccgagatctcaccactgcactccagcctgggtgacagagtgagaccctgtctcaaaaaataaaaataaaaataaaattgtataaaattacaatgaaataaattatattcaaaacaaaaggaataaatactcaaaattcatcacttcgtaagtacttcattttgctgttatcagtgctcctggagttatttgcatctgttctgtctgtacggtggaaatgctctacaattatgtgctccagtgcatctctgccttgtcagagggtccccaagaccaccccggctctgatattcactgagaggattcagcatgccgtcatattcctggctatgacttattatagcaggaggatgcaaaatcagcaaagggaaaagatacatgggagcaaagtctggaggaaccaggtccaatcttccagagacttctcccagtggagccacacagtgcatgcttaattcctccagcaataagttgtgacaacacaagtgatgccttgcctaccaggaaagcttgttagaggctcagcgacagaggttttcttgggagctcatcacataggcagcctctactagcatgtgcctaaagtctggactcccagaaggaaagcaggggttcagcataaactattgattgtacagataatttaggcgcagtgagctgctccatcatggagcactgggaacccttctgaagtacaagttcccaagggccagccttgtaagcaacccctacagaggacagcagtccgggttgctgtgctaactctttcctgccttcttcccagcttcacattcagtgatgcaactttggtggcctggaagtatttacaccacagaaattggcaaatgctataaaccagggcttttccccctacacggaactggttgttaaagattcaccagcacaccattgcttaaggatgcttaaggatttcagagagaggatttggccccaattgtgccagtcatttgctctgactgtgggcattggagaccagcatgaagaatcccactgatggttatcctttctccccatggggggttcccagcctttccctcactttggctcctcttcatcttgcttctgtacaggcaccaggagaactgtccagatggaccacaccttcttccctggctacagaaagaccctgctgagcccggaggagatactgctctccatagagatcccctacagcagggaggtgagatgttggaagtggttccaggctctgctcaaataagtcctattgccaggctctagacaaagagatcagaatctgcttcagggaccaacctgagcatctctcaacctttacattgatgatttttctctctgaactttgcctctgttgaaccaaccatactcctttctggacaatgagaagttgcaggataggagtccacatggcatggactgtggaaccagatagctagaaacttggtcctgagtgagccatttcacctttctgagccccaattcccttatctgtaaaatgggcaccatgtcacttactatacgggttattgtgaggactgaaaataaacaattcataaggttcctggtgatgcctagaagccagtggagatgaccagcagctaagaacagaattccaagaacacatctagctgtcgaggattcctgcccatgtcagaggaaatttgctttcccgtggtaaagaatgtggggttcatgtgctgcatgaagaagctgcctgtggctcagaccctcatacgctgaccttaggcttgacgaggtgttgcttcactgacctcgctgtggattggtgcggatggattcctgcttcatgagtggctcttctggcctgttggggcaggtctgggcaccactggctgccttcctctttgttccctgggccctgtgatgtcttgacctggtgtgggtctttcctcccttagcagctaccagctcagaacacatccaaaagggcctgtcagaaaaggcatctgggagaggcttggggagccactgtcagagagagtgtgaaggtttcccacagctggagctttagtgagtgttctaaggcatgttttgcccttggtaattttgacctccttaatgagaatgctaattaaattagagcatttatatccctgaggtggtttcttaatggttttctagcttgtctgtatagaccattttgagcagtgattcccagagttttgaatttcacagttcagtaaactttttttttttaattatgagtgatgaaagtggggaggaaacaggtgtagctctattcagtttttatttgccaggtatggtcattaacaaaacacatccttccatgaaagaaaggagattttaatacccagaattagaaactctcaagaaagggtattgtcagcctgacactgacagttcactgtaccccagcgagaatgtccccgtggatgggcacagtcctgggagtgagcacctgggacctctgttccagaacaaaggtcaaactctgggctcagtgccccagagccacacatgacatgcaagctgagctagctttatcatcaggagcgggctccatggcttcttcagggaaaagtgggtggccatttgggggcctcaactgtgggcctcattgtgctttttccaatagatctcctctccctaaacctgctaattttggtgttcatcagaatttgtggtgccagagtgagtacagacctcaaaaggaacctgacacagaaagggcctgaagtcaggtggctctttcctggaaaatagctcatgctctcctcctatccttaaagaaagatcctggctcgaacccaggaaggaaaatgtgtttaaagcacaagggcagtgattgtttcatagtgtgatagtgatattttctttcttgcttgctttcttgctttgcttgcttgcttgcttccttccttccttccttccttccttccttccttccttccttccttccttccttccttccttccttcctttctttctttctttctttctttctttctttctttctttctttctttctttcttcctttctttctctttcttctctttttttttgagacagattctcactctgcctgctggcccaggctggagtgtggtggcatgatcacggctcagtgtggcctcaatgtcccaggctcaagtgatcctctcacttcaccctcccaagtagccgagaccacaggtgtgcaccaccacacctggctaatttttttttttttttgtagagatagggcctcactatgttgcctaggctggtcttgaacatttggttttaagtactcctcccacctaggcctctgggattacagacgtgagccactgtgcccgggttctttcaccaagatttctgaacgatgccaacagactggtgggagaggcaggttagagagggcactgtggaggccacaaacagaggatgccaaaggccaaacagcctcagtccccagagaggctgggtgggcactgatagtgggcaccagggtagggaaggtaagggggatgaggggtctctaaggagcctttgtgaaagcacccttcttcccctgcagctgttgttcaatggagaccacccagttctactttgtccctgaccctcagcttttctgactagcagggagaaatcctgttttttttttcagcttctgggaatgttatctggaacctctcctaaacaggtggtaatcagttacatttgatctttgattcccggggccagttcacccactgatgtgtgatggtgttatcccctcacctcacctcatagccctgtggtttgggtctgccatttgcagcccctacagagcaaggtggaaaacaggcaccttcctgatctcttttagggggagtatttctcagcattcaagcaggcctcccggagagaagatgacattgccaaggtaaccagtggcatgagagttttattcaagccaggaaccacagaggtacaggagctggccctttgctatggtggaatggccaacagaaccatctcagccctcaagaccactcagaggcagctttccaagtaagagctaacagcacaatgaaactgctgctaatgttgctgctactaatagtatcattgttactgctgctgctactatgactaccagtagtattactattgctgctactactaccataactactgctactgctgctactactgctaccacttctacttctactataaccacagttgctactactactattaccgatactactactagtattactactaggagtattgccactgctatgctattaccattgctgctactactattgttactattattgcaactataattaatattatgattgctactactccagctgcttctactaaaattactactgttactcattattgctattactatgactactgttgctactactatcactgcagctatgatcactgctgcaattcctactgtcactattatgattagtattattgctgttattactattactattgctaatactactgctgcctcttctaccgcttactctattagtactactactactattactgctgttatactagtattttactaataacattactgcttctaatattattgctactgaaattactactactactactgttactgctactactactactattactattgctactagtattgctgctgctactattactactaacagtagtattactgctgttattgatgctgctggtgctatgactgctactttttggcattgcttctgttactactattattactactgttgctgttgattctatgacaactgcttctatgactattactacttctactactactatctatcttgttaccactactgcaactattattgctgttgctagtattattactagtagtattgatgctgatgctactactactgctctgttattgctactattgctaccactatggctgtgattagtactactgctactactattactatttctatggttattatataacagaaattgtaccgaacacttcactaacattatcctaggaaatccttacagcacccacataggatcaataaagcttaacttggagtttggttccttggttccgaaattcctggctttgtcatttaccaaatagataacataagtcaagttacttaatctccgtgtgcctcagttttctcattggtaaatgggtgtaggaattttgcttactacattttcaaatttaaattaaaagtgagaattcatataaaacacttaagatgttgcctggcacattgtgaggacttgatagattttaactgttattattttggtggtcttttaaaattgtatctatttttgaagatgaggaaacaggctcaatgagattaattaaagttgcctaaagtcacccaactctaagtatccaagccaaattttgaaccaagctttgtacagctccgaggccctgggcacacagctctacacaaaggggtctggagggctgggcgggagcgctgtcccacgccctgctctctggcaggctctggaaggaggagctgctgcaggacgtgtgtgcaggactggcagaggagctgcatctgcctcccgatgcccctggtggcatggtggacttccggtgcaccctcaccctcagcttcttcttcaagttctacctgacagtccttcagaagctgggccaagagaacctggaagacgtgagtggggcctggccaggcacaggctctctgtagcatgtttggggatataagaagttggtctgggtagtaaatttgctcctctcctctggacatctgagggcagaatagcagggaccagggtcacagcacaagaatcttggaaatgggagtgaccagctggcttagaggcagagtgctgggctgggctttggcgacagaggttcttgccctgtaatctcagctacgtgggaagctgaggcaggagaatcacttgaactcgggaggtggaggttgcagtgagccaagatcatgccattgcactctagcctgggctacaagagcgaaactctgtctcaaaaaaaaaaaaaaaaaaagaaagaaagaaagaaagaaagaaagaaaggaaaaaaagaaagggaatgcagagtgtgatgagtatgtagggtggaatgggaatgtcaggtggggctgcaatagccaggaggccgtggaatccataggatatgtcttcaagagagagtgcaggctgaggtgggcccagctcctgagatttgttcaccaagaatttattttgcacctatcctatgccaggttgcatgaggtgctagggggcagctcagtgtcggggagaacatggacaataaaggccacacttgggagacaactgcagagatggagtgtggagggagcccaaagtggagaggatgtcactgatgcagggacagcagagataaatccttctttaagaaaagaactttaaaaatacaggattggataaagaaaacctaaaattactcttactctcaaatcccagagaaaaccactgccaagtattttggttatttcctctgattttttttcctgcatttatctgtccatctatctgtcatactgtttgtctgccccatctctctctctctctgtttctatctgtccttccatccatctatcaatatctgtcatcttcctatcttttgatgtggatatattattttctaaatagacttgggaccacactgcatatagcgctttaaatccccctttgcttccattgggattatattgtgaacattttccatatcattaaatgcttcccacagaattgaggagggcaaggaaggaggagaaggatactcccaacagagacagcagcaggcataaatgcaagaaggagggaaagagtttggggtgttggagaaagtacagaccattcagtctacagggtccactggggggctagcagtgaagctgaggctgctcttaaattctgagctctcagagcccgtgtttggatgctgagagatccagccagcactcaaaggcgtatgaagtgaagagacaaggggatcagttatcagtttggtcatttttatggacaagcaaggaaggaatcagtgattgaaatataatcctggtcacctctgttttcttgtctccacagaagtgtggtaaactggaccccactttcgccagtgcaactttactgtttcagaaagaccccccagccgatgtccagctcttccaagtgagtacggtcagtgtatggctaatatcaggggactttgcaggccacatgagttggctagtggctgaccatcggctaatgactaacttgaattgtatataggtccataaaactccttactcttgtgcgtaaatacccagtgaagaaaatgggaatcgtatagatgttaatgtcactatttctcccttgtttgcctaaatttctctctttgggattaaactcttagctgcccctcttccttacagattttttttttttacgtgatggataatgtacaaagccagacgtcttcttgagtatgctaaaacaaacaaacaaacaaacaaacaaacaaacaaaccatctatctgctccagctttttgagaggtgctacgtaaagggtcagcaaacttttcctgaaaacattttagattttgtgggccagatggtttctattgcaacttctcaactctgtcattgtagcacaaaagcagccacaggaaaaacaggaatgaatgagtgtgttgccttccaataaaactttatttacaaaaacaggcagtgggcttgtgttggcccccatgccacaatttactgccccctgcacactagagtgtagtcgtcccaggcatgaactctggaacctgactgcctgaatttgtgtccacactctgccatttcctaattgtgcaaccttgaacaacttacttattcccttgtgtgcctcttaagcaagttaattatacccacctgcaaatgtggataatcacatacctgtttcataagatggttttgaggattaaatgtgttaataaaagtaaattgcttagatggtccctgggatatggtgaaacactcaataaacattagccattgttatgattactttgtaaaacaaaaacagaactttattatgagatcaataatttaaaacattttagaaaatacagaaaagcataaagaagaaaataaaaatcaccaaaaattccacctccgagagcaaattattattatatttttgtttaattcttttcattatttcctatgtgtatatggacatgtatatactatatttatagcatatattatcttttcctacataattgaattattgtcaaacacaaacacaaatgttttgcgtcacgtttttatgcttgcatgaacaatccttctaattaaagttcttcaaaagtatgttttaatggttgcatactattgtgttatatggggctgcataatttatttacccaatccccaattagtggaaatgtaggttcttctttttcttgttgttcttttatataacaacactaggaccacccttgctatcacttatgcaccttcctgttccctttgccccacgcccctctgaattctttattcttaccccttggtgtcctttgccatgtagcatgaagctcaccagtgtcccctgggcccatagaggggcagtgtccttggcagctcaccctgattgatgaccctgtcattccaggaggtgcccaagggtcagtctgaggaggacatggtgggccggcccctgccccacctggcagcggacatgcaggcctctggtgaggccgtgtactgtgacgacattcctcgctacgagaatgagctgtctctccggctggtcaccagcacccgggcccacgccaagatcaagtgagtgacaccgccaggaggagctggtggaatgctgtgagggggccactgggagtacccaggagacttctctcaccctgctaggccatggagatgcaaagataatgaggtgcctccaggacctcccaggctagagagaggggaagaccttataggcaggtcctattattatctctctgttccagatgaggaaactaaggcacaagcagggtatgttacttgccccaggccacacagttggtaagtggcagagttggaatttgaagccaagcaacgtgagcaactgtgttgtgctgcctcaggatgttcaagccagagagtgtgaacttcacttcctggtgggcgttagacacataccctgctgagcagatcagctaggaaaagcccttccttgggcccactgtgtggactaggaatcaggtttccttgtcattggtcttttctctgctgctgctcctagtttggagacgggatccctccaggaatggtgaaccaggcacagctgaaatctaacagataaataaacttgtccaggtccctgaaaacaaaaaaaaaagtgtttaagttcccatggttggcagctataggatacattctttcccttttgttccttgtcttgagcccatattgacaataccataacccacattgtctacgctcaaaccacctccatccaagcaatgtgtgaatggatggaagaggatgagggggttgacattgttgtaactgcaacagttggggccttcctgggctgcacacacttggaagtgggtttggcaggacagagcttaaccctcctaacttaacctcagttccaataatcccagtcacactgctttctggcttagtgactttggaaaaactacctaacttccttgagtctgagtttccccaaacataaaatgggaattaggataagtactttcagggtttcttgtgaggattaagcatttggaagatggttagaacttcagggcagtttacatacctttccctgaaatgtgttgccctatttccccatgggggcatgtgccagtgtgatgagtacatagttgagactgttatccttgatcatatagtcgggcattctccctttgcggttcgtgttgcctaattatggtaggatgccctgtcatgctgggatagcacaatgaatgtggggagacaaatccagctttatgaagctgttcagggccattggatcagtggcacagccgggctccatgtgctgattttgggagggagttaggtgtgatgtgtgcatgagccagagcgctccagagtaagtgctgttcgttctgatatggcttgcttgtacttggagaccagacagagcatcacgtaacatccaaagaggcctggcctgccttcttcactttatttatttttaaatggagacaggatcttgctatgttgcccaggctggtcttaaactcctgggctcaagtgatcctcccaccttgacctgccaaggtgctgggattataggcatgagccactgtgcctggctggcttattcactttatatccccagcaacattcagcatagtggcctgaacatggtgaggatttaatcaacatttgcagaataggatataattgaattagaatctaatagaaaagaagcagagaatcgaggaattaggatggagccaagccaatcaaaataagaacagggttgtccaaccaggtgatgggtcccctgctccagcttgctgacctggcccctcacaccactcattcttgcaggtccatagatacatcagaagctaagaaggttccagggtttgtttgtttcatttccgctgatgatgttcctgggagtaacataactggaatttgtaatgatgagacagtctttgcgaaggataaggtaagtcttggatttttttatatgaagtaaattgaataagtattgaagttttaatttgtgtacattgctccaaactcctgatctggaaaggttatacatttgcatggattatggccatcatccagttggcccatattcaggacaatccaaaaagatagaatatgttacttgtttatttaattgatggctgctgtttgtacatggcaggatgtctctatgccaaagtggcagtaaaacaacactggtgattgcatttgaactaacaattcggttcttaagggacaacagtaagctctttaattaaaataaaataagaatacatttagtacactaactagaaatcacaataccaccactacgtgaattggtaattcacttacatgaaagtttatctattgatttcaactatgtggaattcttcaacaacaaacaaaatttttcaaatgtttaagacagctgctacaaaagtccttatattttactcataggatagttctcgacagtcattttgtaattccaatagacatccaatgttgcaaattagacatgagtgggtacttctgaagagtgaattcatcttattattgatgtaaaagtgaaggagataacaaaaaactaaaaatatgagaacaaagactccatgaggagagcatttcatagaagaacaaattgctttacatactttaagagcttctcagagcagtgctagcattatggctagtgaaagtcagccagatttaaccaccattgcaataaccaacagccagatttaaccaccattgcactaaccaactgagtcaatagcttatttgggccataagagataattatcagagacatgcatactaattgttggttcctattgtcagaaacttcatcttctaaagtgatgtgttgtacaaaaattttttgttgtttactttttcggggatgttggatgacaattatctctgaaggagatccattaagcttttatgaaactgaattattaaacaaaataaaacaacaatcattatcatataaatttttaaaaatatttttatgggtcatcaagtctaagaaccagtatttgagagctactgcttttgggtattagttgaagtataagacatgatacttaaagtatcatgtcttgtaagtacgtgtcttacaggccttatattgatgcttacagcctttacagcctttgagaagcttttggtataaatctgggaaatagaacataaaagagagtggtgaggtgaaaagaaaataaaactgggggtcagggcctaaggttagtcatgatctgtcaatagtaactagccaaatgtggccttgccttctgagtctccataggaagaccaatggtgttggagtgaatccgaattgacaaatgcacttcctggtgccatatccccctttctgctcatggcaaacaaggaatcaattgccacattctatctggttcagcttctcaatagtctgccagtactaaatactcagaattggcccttgagatgaaatgtgtttgccatcttgatcctagtgatcctcaatcagtaggataaggaaattaagaggccctagggcattcctaaagtgtgctttgattgtgagggcagggacttcttttggctttttgaacaatccaaaaataatttgtttcaccatgagcctggttgcgttctttaacagtctgtctctgctactaggttacttgtgttgggcatatcattggtgctgtggttgctgacaccccggaacacacacagagagctgcccaaggggtgaaaatcacctatgaagaactaccagccattatcacaattgaggtaactgagaatgggctgcctgtgtagaggagtgagctccccgtccctggatgtgcatgtattaggatcccctggaggggatttgagcagcaggtagggatttgactagatgggtttttaaatccctttcaaccctgggatttggggtcccatgaaaccttcccctttatgatcttatgcccagtccactaacaacagccaaactgagttcctccttctcaaaattttacctccatctggtccttgtggtattctttgcttttcctcaaggtccagggttgtccacctacagctgaggaaatggaggaatatgattctctgggctatagtagagcttacttcaagtctgtatgtgaagcatatagagattcatttatggaaagagagagtcaacaattcagagagcttctctttccctaatcagaacctatctttccctccctgctaaaagccacatttctgaaacttctttgtgatcctcaggatgctataaagaacaactccttttatggacctgagctgaagatcgagaaaggggacctaaagaaggggttttccgaagcagataatgttgtgtcaggtagagctgttccatgccgcagtgggaatggggtggccctggtttgcaaattaaattcaagagatatgcattgaaccctgaagcagggaagtgatttctggacatttcctgacagaagattccctcttacgtttgtagcaaaataggagacagttggatgggaaagagcttcagttatcttaatctctactctcctgtctctaggctggacctaatgattctagagagattctagagtattctgatccggaagaccaactagggatgataacagccacatatgcctgacagccttaggtctgccaggcctctaaccgcataaattctcatcctccccctacctgaaggcagtccttacccatcttcaactagcatcctccctcctcttccctttgccctgctctccctccacctctgtctctgacactgtgaaatcaaatccattgctcccaccccaggacaacacagctgactacctcttttggaactgttttttacattggctgttgtgctttgcaaagtcactatgaagccaactctccaacaagcctggtatgaagccgactctccaccaggcctggtagcttaggttcgggaacatgcgctggttttcagtgtttcaatcacatctattaatccctgctttactttttccaaaattttacaatgatggtttttgaaatccttgatttccataagctgcagtgagaagagaactttggactggatgcatttcagtgtcgttctttctttgttgtgggggtggggatgaggctggagcactcagctcccagatagctaagactttgggatccttttccaccccaagtgggaaaccttgaggttacccctcaggtagccattgcttcctgcccagaccacttggcagagtatactctctccttaggcaagctggccccttctgcagcattgggctcactgtgagggatctcactgcttccccaggggagatatacatcggtggccaagagcacttctacctggagactcactgcaccattgctgttccaaaaggcgaggcaggggagatggagctctttgtgtctacacagaacaccatgaagacccaggtagatgccttttgggtcagctcaaggggctgtcagggtagcggggagctcctagcagaccaggagggccatgtgggaaataggaagagggagatgtgggctggtccagagggtactcgagtgtcatagtcctggagccagactctttcaccaacaagctggtatcactgaccttgttccataacctttctcagcctcagttttctcatttataaaatggggcctgggtgggttgtcataattattagatgagttttaatatttagactagagtttggtacatgttggcagttatcatttcagtgcctatggcctcggtcaagtcatatcctgcctcagtttcctcatatgtaaaatgaaggtgttggattaagtcatagccacagggaccactaaattatctacagcagctcaaatattctttgtttctctactgcctgtaatgtgacagcggttgccagtgttttcggcagctggtcctactctcatgatctaccagtcgctggtcacctggtcagacagaataagtttctaaaagtattctgtgaccaactgcttgccctctggttaaaaaccaatgctgaattcagtctggcaggccccagcatcactaaccacaaagctgcatgggattttcaatcaaggaatttcagaattcgcagcagtaattaggtccatgcatgttggacaggctctgccttaggcctgaaataagttcaggtaatgcattcagtgcgaatgttctgtctttcactcacagagctttgttgcaaaaatgttgggggttccagcaaaccggattgtggttcgagtgaagagaatgggaggaggctttggaggcaaggagacccggagcactgtggtgtccacggcagtggccctggctgcatataagtgagttccaaagcctgtttctcccatctggctctgggaagaagaataaggctgtctgtgccaggaaggttttcaggacttccccctgttagaattagtttccccctgctttgaattctcacagccctctgtttttagaacccttgggcatagtgggagggtaacatcgcctgttccgttgtggtctggcacaggtatccacctctgatctctcctactggctgtgagctctctaaatgaaagcacatacctcgcttccttatattctccaccttgtctatcacagtgcctagatggcaaagcagccctgtacaggtctgctcagcgtgcaggtggaaaagatggcttcccggctgtttttattctgagacttgcagggctgagctgctgttgaaggctcacaggcttctaacactgtcggtaacccccaggaccggccgccctgtgcgatgcatgctggaccgtgatgaggacatgctgataactggtggcagacatcccttcctggccagatacaaggttctagatgttggggctgtgctggggcgggatgagccattttgtgaggaaagctgggaccgaatgtccaggcatcctgagctgcactcctactttttgcctatctgtagaagttcaaataagatatagcaagaagagaaattcaaataggctaacagcaacacagaaaggtagtgctgagggaggctggcaccccatctcccaagggtggcattcgtgacttctgtggatttgctgtgttctgagtctggtcaggctcagaaacatgattcaccagcatactcagggtaaaccatcgcgcttggtagcccaacattaacatgatcacaggcctgatgtaaaaattggaatgacagcttagtaatttactgtgagaaataaaacttcccaggggcctggacaggcccaagatgttaggatgggtatgtacttttccagaccatggttggacacggtccacacttctctctgggacagtccctcttctgggtttctgtggatgaccaatgcaccctttccctgtacacatcagcaaagccttatggggctccgcctagctctagaaacctgagagccctctctccatgaattggccttgaacatggcctttgacctattctgagtttgtttcatgatttaactcccagcaagggattatatttctcttagccccaaattcttgtgggcctgaagaggattccttgatcttctggggtcccagtttccgtgtgtgtaaaacagcctgtaaagggtattttaacagtgtacataaggtcaaggatttgcgtgctcctgtgaaatgaaactttttgtgtcttctgaccaaaatctgtgaggagacagtccggtatctttcctagtttctcttccctgcgcttcccccccgccctatcgtgaccagccttctctttccaacctccttactcccctccgagaccagaagccaggtgccggtagcaggaggaagaggtgtggggacaagggcttccaggggtggggagccaagtgtgtgccactggggagaggtcagttcctctcttcttcacttgttaccttctggtgacttctgaaggtcacctttttttcacgggatgatgtggtgttctgtttatttcctccaggtgacataagtgggagggaggatcagagggagacagagaggtgggggaaacgggacttatgataaatccctctcctttctccttttatcaggttggcttcatgaagactgggacagttgtggctcttgaggtggaccacttcagcaatgtggggaacacccaggatctctctcagagtgtgagtaggacctgcagccgcccctgcacccagagctgacttcgaggtcagcctgttcccacgcaggcacaggcctccccagtccctgcttgctactgttgcagtagtctcctaatgggtccccaccttcaggtttcccatcctcgattaatctctatagcacaatgatgatcgtattatttctctggttaaaaacccatccttcctgttcaagataatttgccaagctagcaagttagttacaatgttttatgtatagtgtggtggcattgtgtgtgtgtacatatgcatggaatagtaatatatatatacagtaacatataaactaatactaatatatattactatatatagcatgcgtggaaaagtgtctgaaggaacatataccaaactgttagcagtgatgatcaaattgttaacaacgattggaatggtattgagaggcgtacacactgtcattttctgtgttacacatttctgtgatgttttaattttaaataattcgaattgctttttaaaaaatggtttcattgaagtataatttacataccataaagttaatccatttgaagtatataatttaatggttgttagtaaatttcaagagttgttagaccactgtcacaatccagtgtttgaactcccatcaccccagaatgatgtcttgcatcatttgtagtcattcccattaccattccataggcccaggtcatcattaatctactttccgtcttgtagtcaagtcattttatagaaccatacactatgtagttttttctgtttggtgtctttctctcagaataatgttttgtaggttcatctatgtttttattattattctttcttttgagatagagtctcactctgtcgccaggctggagtgcagtggcacgatcttggctcactgcaacctctgcctccagcttcaagcgattctcctgtctcagcctcccgagtagctgggattacaggcatgcacccctatgcctggctaatttttgtatttttagtagagacagggtttcaccatgttgatgaggctggtctcgaactcctgacttcaagtaatcagccctcctcagcctcccaaagtgctgggattacaggcgtgaaccactgcatccggcccatctatattatagcatgtatcactacgtcactcctgttcattgatgaataatattccatcatagggatataccatatgttgtttcccattcaccacttgatggacatttgtgttgtttccagttttttgcttttatgcataatactgctctgaacactctcatactagccttatgtagacatgtattttgatttctcttgactaaattcctaggaatagaattgctgaatctgtggtgagcttaacattttaagaaatgatcgcattttcatgggcggaggcggcccggcaggcactgggagagctgggctttattattactaatattctacaattgtttaaataaaaggaactttataatgaaacagtttggaatactggctcaagaacctatgtcaagatgagctgaattttagtaaattactttaggaattatgacgagctaattgaattaggcttgtgacaatgaggaatttacatgacaggtaaaactcatattaaaaatgtttagatatttgcttctatagatgtcactttaataaaataccaatttagttttacttgtggcttatctagttagtaagaactttaataggctttactgggacaagcttactgctcttgtaggagctcctctcacaagtagttgtaatgccatagagtgacactcaggatcagtagcttgagatgttatggtttttctcttcatctatgacttgcagagagcctcccctttttggatccagacatcttttctgaatcctggttccactagtacatctgctcccctgtgatcccaaatcatagtcaatggtgcctcgaacatagcaccttcagttaaaggctgcctagtgctctgaggaaagcttgctgattatcttcctgctctactcaccccaaaaggcagaaaagcaacatagtcagtcctgtgctcatttgtaattgtaaagatcagttatatatatatatatatttgtaatgagagcaagtatatactcattataggataaatttaaggagtttaaaatatcccaaagtagaatttctatatctagccttttgtttctttcatgaatatttgcaagcaattaccattaaattcacacatgaaagattaatctgaaagatcagagagaccgtattattcctgaccatgatagaactgcttctgtggtctgggacaagtaacaaagcatctgcttgagttttgtttgtaaaatacactattaatatttgacctacctcaaaacatattgaggatctgtgaaatgctaagtgtccaaaataaaatattgctgattgtctttttattgaaagtaaatttcctcattaagccaacctgccttctgtaagtcatagtgcttaaatctcaggatttttcattaggagagacctgtcattaaggatttgtaggtataattgcttagcctccattattggtgcttgggatagagaggttttagatttttctgttttttgttctgcttcaaagctcagtttattgaagacatttataagctattggatcatcacttgaatcaagattttgactagtgagcttaattgtccatttcttatgatttcaaagttacagtctgagaaatggctgattttaataactgtcctttcataaattaatgttggaataaactgaagctggagataaatacttcatgaaaactggctaaagtctgaaataattacctgttttatgtccaatagctactaaatttgatagaacagttttgagaaacatttcattcttgaaggcaacatctgacatggttctcagcaagttggaatagtaaagattgctgggctgttacgatgagtggaaggagcgtgtttgtagcatacaagttattcagcaatcttgtgctgttgacctaaaaatatgtcttaattcttcatcaaggcaagcctggtgaagtctttacttgttactacttcaacagttggaattagttgctctttttacttgatgaaacaaaactatacctctgaatatttatggatactttttactaaatcaggcttgtgttcttaatccaaattagtaaagttttatggaagctgaaatgtaacacagtagtctccccttatctgcaagagatacattccaagactcccagtggatgcctgaaacatcagatagtactgaactccttgttatacagtcatctccccttatctgcaggagatacattccaagaaccctagtggacacctgaaacctcagatagtactgaaccccttgttatacagtcatcttcccttatctgcaggaggagatacattccaagaaccctagtgggtgcctgaaacctcagatagtactgaaccctttatagactatgttttttcaatctgacaaccaaggcgggactaagcggctaaggggcaggtagtacacagtgtggataagcaggacaaagggaagattcacatcccgcacaggacagagcaggagatcatcagatttcatcactcaggacagcttgtgatttattttatttattttttgttttgagatggagtctcactctgtcacccaggctggagtgcagtggtgcagtcttggctcactgcaacctccgcctcctgggttcaagtgattctcctgcctcagcctcccaagtagctgggattacaagtgccctgccccaccacgcccagctaatttttgcatttttagtagagacggggtttcaccaggttggccaggctggtctcgaacttctgacctcgggtgatccacccgcctcatcctcccaaagtgctgggattataggcatgagccaccttgcccggccgacttgtgatttaaaacataaattgtttatttctggaattttccacataatatgtttggaccaaggcttccttggttaacaaactacggaaagtgaaattgcagataaagggggttactgctcctgtgctctaaaactggtgtttgggtgatcaggagcaggtagccaatgggaagagcacatctgagtgataactaaaggagtttctctggtggccttttcacattctccaatgctcaagcatattttccacttaccattttctctttcacctcattttgcctcaccatcccccatccctgcttatttcttaagcccattgatggcactcattaaattgtgtttagggctaatgaatcattgttccttaatatccttttcaatatgccacaatttaggacacatttaaaattttctaaaacaatatcctaatccgaatattgattaatttgagccacattcccaactctaactcagggcacactgccagtcttccccaatatctcctctcaattccccaccacaccttataaaattggaatcaaagaaatctcactctgtcattgtgaatctaagaataaaaacactgattttaatactgctttactagtttcagccttctaagtaggtaggcctctaggtattctgcagatcactggtggtcttgatagccattaatatatgtttgtattatgttattttttaactaaatcacagttggaaaaaaacatctttgatattatgcccttggacctgttactgcatcactggcgcttgtgatgcaataggacacttcgcctgtactaaaagggccaagagtaaatgccttgtttttttttttttggtttttgttttgttttgttaaacatgtctatagagttggcagttaatgctgaatttgtcaaacagcccttccaaaactattcttgtatttaaaaaataaatggatctacctaatttctattgattttttttaaaaaagaaatgatcaaattatattcctgagtgactacaccattttatattccaatcaacaatctttagggattccacatcctcagtaacacttggtattgtctgtctttacccactagaattagtgggtgtgaagtgatagctcattgtgatttcagtttgtgtttcttatatcctgtgaccttgcagaactcatttatgagttctaggatttcttctgtagattccttgggattttctctgcagacaatcattatcatctgcaaatagggatggtttttttcttcctttccaatctgcatgttttcatctccttttctttacatactgagatggctagccagaccttgcagaactataggtcatatatccttcctttgtttttattcttaaggggaagacatccagtctttcaccattaaatatgttattggtagggttttgtttgtttgtttgttttttgtagatactcatgatcagattaagaaagttttcttccattagtgttttctgagcatctttataatgaatgggtgttgaattttgtcaaatgctttttctgcactgactgatatgatcatgtgattttttcttctttagcttgttaatattgtggattgtattcattgatttttttaaacactgaaccaaccactgtccttggaatgaatccagcttggtcatggtataattaatatattgccaaattttatttgctaaaatttgttaaggatttttggatccatattcatgagaaatattgctctgtagttttttcttttaaaattataccatcttggtcagggtttggtgtcaggataacactagcttcataaaatgagttagaattcctcttctaattctagaacagattgtgtagaattggtgttaattcttctttaaatgtttggtagaattttccagtgaaatcgtttggacctggagacttcttttctgatagccttaaaactattgattaaattgcactgtggtctgatagacagtttgttataatttctgttcttttacatttgctgaggagtgctttacttccaactatgtggtcaattttggaataagtgtgatgtggtgctgagaagaatgtacattccgttgatttggggtgaagagttctgtagatgtctattacgtcagcttgatgcagagctgagttcaattcctggatatccttgttaactttctgtctcgttgatctgtcctagagcttaaaagtataatacttgaaaaaaaaagaaacaaacaaaaaaacactattgattaaatttccttaatagttctaggactattcaaattatctatctcatactgaagtggtagttttgtattttgagacattcatttaaaaatgttttgtttttcattattattggtacatagtagttgtatatatttataggatacatatgatgttttgatacaggcatacagtgtatcaggataaacaaatcagggtaacgtcaatatctatcacctcaagaatttatcatttttctgtgttagggaacatttcaattctactcttatttatttttaaatatacgataaattatcatatatttaaaaatattatagtcaccatattgttctactgtatgctagatgttactcattctaactatatttttatatgtattaaccatccccattttatcctctgtccccctaccctccccagcctctggtaaacatcattctactctttatctccatgaattcaatttaattttgtctaagttattaaaaaattcatgtgtgtagaatcgttcatagtgtgttcttatttctcttttgatgtcttcagggtctgtagtgatagcccgtgtttcaatcctgatttggtaatttgtggcttcactttgtttttctgtcttgccagaggtttgtcaattttattgctctttttaaagacccgggtttttgtttcattgattttctctattgtgtttgttttcaatttcactgatttctgctcttatccttattatttcgttcactctgtttgctttggatttattttgcttctctttttctaggttcttgagttgaaagcttaaattaactatctgagaatgttttcctcttttttaatgtattcaatttagtgctgtaaagtttcccctcagcactactttagacaggtcccataagttttggtatgttgtatttcatttttttcttgtatttttggtctgcttgtttatttccctgacaccttctctttgatccctaattatttagaagcatgctgtttggttttcaggtgtttatagatattccagttatctttgtatcctgaggctttctattgatttctagtttcattccgttttagttgaagaacacagtctataggatttcaattctttgaagtatgttgaggttcatatcatggcccaggacatggtctgttttgatacaagttccacgggcacctaagcagaatgtgtattttgctgttgatggctgtgtattagttttctgtggctgttataacaaatcaccacaaattgggtggcttaaaataacgaacatttattttctcacagttctgaggccagaagtctgaaatcaaggtgttggcaggaccgcacatcctctggaagctctagggaaggatccattcctgaccttttgtagtttctggtggctggtggcactccttgacttgtggttgcatcagtccaatctctgtctttgtcttcccatcaccttctcctgtgtgtccattcttgtctacctagacatacttctctcttacagagacatgtgattgcatttaggatccactccaatggttcaggataagctacttctctccagatccttaactttatcacatattttgccatataaggtagtatttacaggttccatgattagaaagtgaatatatctttggggaggcatttctttttcctaccacatgtataatgtgctataaatatccactagatcctgatggtggtgttgagttcttctatacccttcctgattttctgtctattttttgctaccaattttgagagaggagcattgaaatctccaactgtaattgtggagttttctatttctccttttagttctaccagtctttacttcatatctttttcagctcttttatttggggcagacacaattagtattgctgtcttttgggaggattgacacttttatcattatgtaatctccttctctctgtctctggtaattgtatttgctctgaagtctactttatctcatattaataaagccactcttgctttcttttgattaatgtttgcatgttatatctttttccatccttttactttcaacttgcctacattattatatttgaagtgattttcttgaagacagtatgtaattgggtcatgtttgtaaatccattctgctaatctcttttaactgtatttatacaatttatacatcatataatgattgatgtgttgaggcataagactgccactttattttttgttttctgtttgttctatcagcttttcatttctctgtattcttttgtgtgccttgctgtgggtgggttacttggacattttttttaatattccatttttattaaaagtatctcttacatagctttcttagtcattgttcaaggtattatgttatatatacacaacttatcacagcctactgaagttctcattttactagttttggtgaagtatagaaaacttacctccctttatgactctgccctttcccatttatattatttttattttaaatattttctttacattcatttagaaccacatcaagacagtgttttaatttttgtttcaactatcagacaattttaaaaactgagaagaaaagcttactgcttgtacccatgattttgcttatagtgtttttccttccttctaattttccaagcttccttcttttatcattttctttctgtttagaacttcctttagtcattattttattgtgggtctgctggggacacattttcttagttttccatctgagaatgtcttgatttttttccttgttcctgaaggatcactttactgggcattaagttttgagttaacagttcgtttctttcagcacctgaaaaactgccacttcgttctggtctctatggcttctgatgagaaatcctctgtcattctaattaggctccccctataggtaagttgtcatattctctgggtgctttcaagatttttcctttgtctttagttttcagaagtttaattatgataggacttaccatagatttcttcaggcttatcctgttttaagatcaccaagcatcttgaatctgtaggtttacatatcttgaaaccttgggaacttttcagttattattgctttgagtactttcatgctcttctctttttctccttttcttccagatctccaatggcatgagtgttaggtcttttgttatagtcccaaatgtccttgaaactgtaatttttttttcattccatattctcaatatagttcagactggataatatatcttattctattcttcagttcactgattctcttctatgtcccttctcttctgctgttgaatccacccaataagctatttattttggttgttgccttttcaattctgaaaggcccattaggttcttctttgtatcttctatgtctttggtgagactatctgtttatttccagaagctttttatttctgtcaagtgtgttcacaattgatctcagaggcatttttttcatggctgcattaaaactacagtcagataattttaacatctttgtcacctcagtgatggcatctcttggttatcttttattattcaggaagtttgagatcttcctatcacttggtatgacaagtgatttttcactgaaacttggagaaacccgtcattaatgtacagatgatggtggtgatgacgatgttctgagagcttggatctatttaagccttctattttaattggctccttctgaccttgctctagcagtggtggatgccacctcatcactgctaggtagaggtagaaatcttgatcttccacttggcctttgttgatacccaggttaggaacaactccttcttacttctgggcagagatgggagatctacattccacgtgttgtccatggacatcatggtgtgggtggcctctttaccactggcttgtgctaaaagccttgactcttcactagactttttctgacacaaccagaaaggaagggaagagtggtgtgtctttgttatgtgggtggggaagtcaatgtgttctccagtgactccatcataaggtgtaggtacagccttatgactggccatcagcaatgaaagtcttgactcccgatctggtctcctctggcaccatcccagcaggaatgttggggtatctcataacagcctcatgaatgtggaagtctgggctcctactgagcttttgcaggtgtgagtaggaatgagactacgttttctttttctgtgatgtttggctggggtacttattgtctaaaagtgtttagttttgctagactgcccttttcttggtcttttggctagagataaaaggatttggaatatatagtgagctttttggtctctgtcatcattggcatttctggattgccagctcctgccccaagtctgggatatatagatgaggcaaaaaggaaacccctggaactcaccatcatgtcattccttgggttgcaaagtccctactgggactgcctttttttttcctccacttttcagagtattcttacattgttgttgtggttgattttaaataaatatatagaattccagagtttttagtggtacttaatggaagtatggaagtgaaagtctctgtatatacttcaatgttgagtttctcacaagatagaatcctgagtaatgtgtcagtaattatcgtgtgcttttatatttagtctcagttgatgctcctccagtcacattgtaaatgaccagggtaagaccagaagtgggagctacgtactcctcattattgtggtcacagctttgggagatgaacttggaatattgagttgtttagaaaaataactagagggcttggatgaaataaagtcaattgtgagaattaacaaatttattttggtggtaccactttttacagttttttaaatcttaattttgtaaccatagtagttacgtatgtatagattcttgtttgaaaaaaaaaaaaaaagaggtggctgggtgcggtggctcatgcttataatcccagtactttgggaggccgagatagacagatcacttgaggtcaggagttcaagaccagcctggccaacatggcaaaagcctgtctctttgaaaaaaaaaaaaaaaggttaaagttttcaggaaaagccgacattcccctttacccttttggatcacactcctctctgctaggggctgcctaattggctcagttaggaaacttcacacttcagatctagggttaaaattgttcaaaaaaagaattattcttatttcttgttctcttcaagtataagacatgctttctggtatgggaaaaaatcacgttgaaaaacatacagtctttgtccctagacatcctacaagattttcaattcaatccagcaaatatattaagtgcctagggtggctacataaggaaggctatgacattgtttctgccctcaagaaattcacaatatggcaggggaagtagatgataaaagaaacagaaatacaagttgataaagtactagggaaagcaaaagtagaattaattgagacatagaaaaatcatttaggatgagtaagactgaaattatcaaagacacataaagcaacttgtgggagagagagagtacccatagggaaataaaccaaaaccaaaataaaacaaacaacaaaaaaagaaagctgccattgatctttccagagagatgagatattacaacttaaagaaggaacagaatgttactaaaatggagcagtcaaagaataaaaatgagctctcatgacttgacactatgaaagcagaggggttcaaagaaacttgacaaatctcccaaaaatggaaaacaatgtgaaaaaatagagaaaatacaaaaaaattttgaggatcagttcaagaggtccaaatcatagacatttcagaaagaaagaatggaaaaaacagagaaggggaagctatcaataattcaaaaaattttccaaaactgatgggcatacactttgggattaaaacagcccatataataaccagcacaatgaaagaaaatagacctatgccaggttataatgttatgaaatttagaaacctgaggacaaagagaagatttcacaattttcaggaagaaaaaacagaactctccaagaattggaaaggcattgattggacttctcaatagcaggaagctagaatgtactagtaaaatgccttcacattgctgagaaaaactggaaagcctacttccaaattaggattctatatacagctgaatcattaattaaaggtgaaagtagaataaagacatttcagacatgcaaggtgtcaaagatgttcctctttgtgtaccctttcttatgaagatattaaaggatgtactccaacaaaatgagggagtaaaatttaaaaagggaaaggcatggaatctaggaacaggggattcaatatggagagaggtgaagggaatccctgggatgaatccctttacccctttggatcccactgggggatttaagatttaaatccttaggtaacagctgaatagcagatctagtgggcagcgaatttaaattggagtaggccagatggctctaggatagaaaatctgatgtgcttgaacatgttggaagcagagctcaggagggaaaagagaaaatatattaggagcagatttttagaattggggaaaactttaagttggcttagtaataagtacatacaaaatgaagcagatgaaaaaataaagacaattattaactccagggtgaaaaaaatattatcaagagagaagaaattgtaatgtaactacatggttcagctgtaagtaatgtttacagagttataatattaaattgactgttgatctgatcaaaatacaactatactgggaaataggaagctggtaggggtgagggctggagggagggggctaagccaaaactaaattctccttttccataaaagaaagacaacctaaaagtctaaaactgaaaaagtcaagacatagcaatgtagccacattatctagagacgtataagtttatctaaatagctgatagatgataagctatgagaattgtaagtagttgcttctggagactagaaggaaggaaagtggaagaggctactgtttttcataccatgacttacagaagtatgggacacttcaaactatttgcagtacaactttcatggctgtttcatagttttcattaactaatttacaatgaatttttaaaagttaactgttttcaaaaagaggcaagtgattagttccaactagaggctctggaatgaatttctccacagagttgctgaatgaggggactagtgggtggataaggtgcagtaaagatgctgccgcaggctgggctcatgcctgtaatcccagcactttgggaggccaaggcgggcggatcacgaggtcaggagatcgagaccttccaggccaacatggtgaaaccccatctctactaaaaatacaaaaattagctgggcgtgtggtgcgtgcctgtaatcccagctactcaggaggctgaggcaggagaattgcttgaaccagggagtaggagtttgcagtgagccaagatcgctccactgcactccagcctggtgacagagcgagactccatctcaaaaaaaaaaaaaaaaaaaaaaaaaaagatgctgctgcaatatcctgggggagaaataacttgggtgcagaggggaaaggcaggcaatggatacagccctcaaagttcctactttgaggaaccaatgcaatcagtacactgttctctcccatttttgtttcctctgcttcagattatggaacgagctttattccacatggacaactgctataaaatccccaacatccggggcactgggcggctgtgcaaaaccaaccttccctccaacacggccttccggggctttggggggccccaggggatgctcattgccgagtgctggatgagtgaagttgcagtgacctgtgggatgcctgcagaggaggtaagctgagaatttggtagcacaagtcagagtgctttagactacttcgggggacagcggctttgtatcttgggcatctcccatgggggcagatgacaggcaggggctgctgagggggttggaagcttaactttaggttgtgccctgcccccacagtctcttgccgccaacaaggcctctgtgtctctctccttgggaaggtgcggagaaaaaacctgtacaaagaaggggacctgacacacttcaaccagaagcttgagggtttcaccttgcccagatgctgggaagaatgcctagcaagctctcagtatcatgctcggaagagtgaggttgacaagttcaacaagtaagcactaggagtagaagtccagttgctcttctgggtttcatatcctacagcctttctgctgaccccatataggaagctaataattcaatggataacttatgagactgctgagtttgccaactcattcacagccatctctttaagatcttataatgatcttcagcccggggcaggtgggctcacagcctgcaatcggtgtcatagcctgtcagttgtggttcatctttacgcgtttgtaaacctttgggcacaccgtatgataaagaacactggcctaggactaacacgtcacagttatgcacctgcctctgacactgatctataaatatgaatttgggagtccctgggcctcagtttcatcatgtgaaaagtgggcataattgggctggctctgtcttcctcacagagttactcagataattaaatataaagcacagagaaggctttattattgtcagtgaggcacagtgcctcctggagatgatgacagcccaagattaccagggcactctgacccagtacgggttgcactcagggaagaagagtgttgacaagaccccctggtgtgggctgtgcaccagctgactgttctcatctttgtgggcaaccccaaggatgtcctgttttgatgtgaacatccaaagatattcatgtcctcaatatgatagcgaaggttctataagaatattcctttttctctagggagaattgttggaaaaagagaggattgtgcataattcccaccaagtttggaataagctttacagttccttttctgaatcaggtaattgctttatagaattgtgtccatctccgctggactgggaccccatttccctgttgtttacagtattgcaagggaaatttcgttgtcactggtaacagggcttactggaggaactctttgctctttaaatgaaagtcgaacttgcacagtcctatttgtccagtaggtcatgggcagtgagactcagagatctgggatcaacctctagttcttacctttccttaactatcggactggagagagctactctatttttcttgttctgtttcttcacttacagagtgaggacagatatttgacctatttccctttgagaacaagagaaacagagagaaacatatgaaaaccttctgcaaactactgaagtgctatataattaaatcatgagattcatactgccataatttttgtctgaggtttacatccaaaatgctgctggttctcttgttcctgggctccatacctttggtcccaaaccttaatgagttggattttactgaattttgtgtctggcatccttctctttccctaggcaggagccctacttcatgtgtacacagatggctctgtgctgctgacccacggggggactgagatgggccaaggccttcataccaaaatggtccaggtgagcagccctctctggcagtgttgtgtccgttgttagaggaagtggtataagaaattgagcagtgggacctatcagagcagaagcaggcccggggtctggccctggccctgttgcttatgtccgtgtagcctcgggcaagtcttttcccccacttgaactcagtttcctcatctgtgcaaaggagttggccaggtgacctctgaggacattgctattaccaacattctcagtgtgtgaggcgggcatgctctgtgtgtaaaggtcgcgagtttcccacattccaagactcctcggagtggagtgaggacaatgagctggattagagtcagcacatctgcttggcgcatggagactggccagattggatcttttgaaaagtgagtgttagtgtaaaaaggcactgtttgtcagtttatgaattttttatccaaccgaactccaccaagtgctccttcaaattcagccagatgtaaggacatcccagacaatgagattagagtttacgaaattgttttcttgcctgtaacagtgaatgtgaatcctgtcttggggagaaggcagccaagccccatgataaccccttccctagagggcaatgtcttctcttttcgcaggtggccagtagagctctgaaaatccccacctctaagatttatatcagcgagacaagcactaacactgtgcccaacacctctcccacggctgcctctgtcagcgctgacctcaatggacaggccgtctatgtaaggagcccatgggatcccgcagagcagagggccagcccagagctgtgggcctggagagtctccctgggtgggagctagaagggttccttgcaggctctttggctctggccaggcctctggctcccttatccaggaggatgctgtttctccaaacctcaagctaaaagagaggcccaagaatcataatcactgccctcaaagctgggaaggatgagtgcaggatggggagatgaggataccgagggaaagcagaggcttctggaattactccctggtgctggagctagcaataggacaatgggctgaagctgaaatttgattatagaggcaggttggccacactggctcctctgccatatgggggatataatcaaacaagcagtttgagggaagaagacacctctctggggatccctcaaacatttcgccctctgtcagcctaagaattgagtcaggaagtgctctccaagcagggactagtggtggccctattggccaggtcctgtcgattctaccctgggccttgatcattctggagtgggtggccaggctgtttgagtggcaccctgggtacagcattgcttggaggtagccttgctggggccctcgggttttgcctgggctacagaatgcagaggactgatgtgtctggggtgtggggggtgtttactttaggcggcttgtcagaccatcttgaaaaggctggaaccctacaagaagaagaatcccagtggctcctgggaagactgggtaagtctgagctgatccaagtcacgtttgcaaaggagagggaaattgacagcagggcctccgttccgaatccgaacaagtaataggagacaatattttagtcagaacagcagagagaagttgaggtggtttgtgtgattttacaggatgaataaatgctcattctggctgggtccctttctctatagctttcatcattgagggaggtcatttgagggaagtttttgatccaggatcttgacagccttgtgggaatcagggttttctttgcctgagcctgagaggatgggtgcctgctgaacatgcctcatcggattgtcttgactcagtgggtctgctcccacaccccgcttgtcccagatgagcctggttttcctcaaatctccatctcttgagtctgagtctactgagattccctctgtgacagcgtggggagaaaacatcacccaaggacttctatcattcctgtgatttggggaatgacagaacaagcacttatgataagaatgagcccaattctctgacttctagaccagtggcctgaatgtcagaaatctcagagggaaggggcaagggagtctcttgcaggcgggaggctgtggctcctccagcaagtggaaatattaatggcagctctgcatccttccccagtattggagtggtagaactgggcagaagagtaacacatcaaatgtgactgcacataaaccggctcactagaaatcttcctcaatagatttaaagaaaacatgccatttttctcagacccactcatcttactatgccttctatgacctctccataccacgtgtaccaatagcacatgaaagtagcctgattttggtagttgggttttagcatggatttatcaggcagtttaaatgtttttaacaacagtcccgacaacaatgttctatcatctgacctgtggatctgagctttgtcttcaaccaccagccatgtagaccagtgtgcctgggaaacagataaccccaataaatgaaccacagtcaccaacacatacatacacatgcgcatgtgcacgcacacacaaacgcacacaagcatgcgtgcacacacacacatgcacacgcacattatgttaaatgactagtgcaggttttaggaccaagtctttccaagccttagtttcctcatctgtaaaatgaggctaataatagtataggttttctggggttgtgagtataagtaagataatgtatgcaaagtgctaaatagcgcgtgtgacacagtaaatcgtttggtggctgctaatgaagaaggcataaattatatcaagtacaacttaaatctgagtctgtgctagtgtttggggataccaaaaaagcacattatatagtccaagggataggatggagggatagtttgatcaacaggaaagcatatgtaaaagcacaaggtgaacatgtgaagtaccacatgagcgctatagcgtgttgcttttataatggccagttagggagagttcaccaaggaggggagaatggagggcaagtttgagtagttgttgagggaagaaaaggatttgcacatatatagaagaggaaccacataaataaaggtgcagaggtgtgggagatggaagaattgagagagatttagctcttgagtgtggctggggtggccctcgtgtgctgcaaaatgccagagagactgtggtccttaacatcaggcatttgctctacaggcaataaggagccgtggaaagctctagagtagggtagtgacatgcacagtgtggtatgttaagatggccactctggctgggctggggagacaactcaagggaggaagaccagttaagaggtgactacagtgttccaggcttcagataatgaggacctggtgcaggtggtgccagtgacaatgtaaggtaaggtatggaaatgggagtcactagtaaaatgagttgattggcttccagaggaggtgagctcctgaaagcttagtggcaggtcagggttcctgaaaacctcacttcacctgcctgatggccatctcctctcctcaggtcacagctgcctacatggacacagtgagcttgtctgccactgggttttataggtaagtatggtgttgtccttgcccagctcatagtgtgatgtcgtcctaacctcactggtccactccagcaccgaatcctgccaggctgtggtcaggagagagcagctccacttatctcttcaacagacttccgctaatttcagtctaaagatgctctaaaatatgtggaatggccacactgtgtgccaaggggtaaaagcaccagctgtgtgtcagaagacccgacttctagttctggtctcagggtgcacaatttcaccagaggaagtcagtggtcctgctgaacctgttcccctgtttcctcatgggtgacgtattggggccagtacctaaccattcctccctcagtgttctcctgcagaaaccaggctgtgtagtgttatgagttaggcattgctgttagctatgcatccttctttgtactgtgtgtgggacttttttatagaatgagaggaggatttagagcacgtggtcttattacaaaatgcagaatacagtggaaacttattaatacagggagattttcaagagagcgtatgacttctgcaactttaaatttagtagcagcgtgaatagctagagaccctgcccatctgacttagcagctgtaaaaagatatgatcatatttactgtcagtcatcatattttagccgtctctgggaagtttagctaggaccttttctttcacagatatttggaatattttctttcaatatatttggaaaataattcaatgtttctttaaaatatcatgtctgaatgtatgaatatatagatgcataaatatatatatgtataaacatttctttgtatgaaacatttattttccgtatgtatatgaatatatatatgaacatatatatgttaggcatatatatgtttatacatataaggcataaacatatatacacacaaaaaacatttatatgccctatatatgaacatatatatggctaatatatatgtatagatattacactgaatatatatgccttatacatatatgtataagacatatatctgtataaacatatgtacacacacaaaacatttctatgccatatatatgaacatacatatggctaatatatgtatagatattacactgaatatatatatgccttatataaggcatatatatatgccttatacatatatgtatgctttatatacatatatataagtatataaggcatatatatgtattatacatatatgtataaggcatatatgtataaacatatatacacaaaaaaacatttctatgccatatatgaacatatatgtggctcatatatatgtatagatattacattgaatatatatgtataaggcatatatatgtataaacatgcatatatatatatatatatatatatatatatatatatatatatggtgaagaaatgttttgccaatgctttctaggtttttaatatctgacctacacatacataaacacattttctcataaattcctattacatttctggagttcatgaaactaaggttttataaaggtcaaagcagaggagcatggtgaaagcagaggtcaaaggaattggtggtggttttttctgggagaagccttttccctcagccccatattggttttgtttttgtgcagatctggtcatatcagctcatgttattcctgtgtagtgtatagaataacacagagtcagtgtactgtaaactctaggtgtgtgaatacacatgcacaaatgcatagatatacacatatatttatatgactcaggcaaaagtggaattgtataaggtactccagattgaaaattttaaaaatgtataagggagcatttgtttctattcgtatcatttccctcaaatcatttgttgaactttcaaagggaccagcaggtggggagtggccgcttaaagatgcacccaggttagcatgagatgctgatcttattaactttctcaccttgagaaggtaagcaggagaactgtgtgaacagtctaagagccaccacaggcagaaccttcagtcctgaagaccttggattctaattcagtacatcctttggagtgtgactttcatgtccttcgtgagcccagatggatctgttacctctctttctactgtgcagaggctagcatggtactaaacactgctaggctgtacagaatgagtacagaagggctactggcaaaatagaggagggcccagagacaaaatgttctggaatatctgggtttcgttaacaggttcaaattgttcaaaccatggacttttttggagaacagctgaatgcaaaaacttgtggggtttaaagatgttgtgggtcaagctgtacgtgcatagggaagggggtgtgttcatgttaatacatgtgcagtaccttctctttcctctcccagaacacccaatctgggctacagctttgagactaactcagggaaccccttccactacttcagctatggggtggcttgctctgaagtagaaatcgactgcctaacaggagatcataaggtaagaagtttgcaaaactttagtacttagtgggaaagagaagagaaacaactgagttgtaatacctagagctgatagagacaagatttctggttggaaaaataatgccttccattaaggataggcaaacatcctataaagggccacagagtaagtgttttagacttcgtgggccatacagtctctgcctcagctactgaactctaccattgaagtgtgacagcagtcatagacagcttgtaaacagataagtgtgactctgttccaataaaatatgatttatagacactgaaatttgaatttgaattgaatctcatacaatttttacttgtcacaaaatattcttctttttatttttccagttatttaataatgtaattattagcctacaggccccacgaaaacaggggcaagttggatttagcccatggaacgtagtatattgactcttttgtacatgatctacccagactcagattcaaagccccaggtttctgggaccctatctgactactcacgtatgaatgtgtgttattcccagttactgagagatcattgctgctccccacttcctacccggctgctacaaactcagactccagcggggatggtggcagcagagatttggtgggaacatcaacacttctgcctgagcacacccagggctttctgcttgggaacgtactctttggttggcacttgtagtctgagggtcactatgagtcatctgtcatattccaaacatcttttcctcctctaacttcttgtaattttgctgtctactctagaacctccgcacagatattgtcatggatgttggctccagtctaaaccctgccattgatattggacaggtgaggctcagtgggaccagggtggtgacagacttggccaccgatgcaccccattcccctcctggggccacctgccacaacatgttgtccaaggttgtcttcaaacaggctgaaacatggaacctagggagtttcccccaaagtagaatttagattggtgatagtccaaccaaccctcaaggcagaaagcaaattaatatgggtcaacttgggaagggcactcacaactcaaaaacaagatgagaaatgaggaaacatggcatgcacatgtgaacatgcacacacacccaatttgagtgtgagcacaacgtagaatggggattgagtttgggatcaatgcccagacagtggggtctgaggtcagaagagttttctgtcttctcattcctcctccacctaggacagcttttgtaccccaggaagcagatgctgccaaacatggagtggtggaaggccctgggagcaaaacacaaaggcttaagcagaagtagtaaagggaggtcccagagaaaatcgctttaggagaatatgggattactatgaagaataggtgaaatagggctgggacctgtggctcacacctgtaatcccagcactctgggaggccgaggcaggcagatcacctgagatcaggagttcgagaccagcttggacaacatggcgaaactccgtctctattaaaaatacaaaaattagctgggtgtggtggcatgcacctgtaatcccagctactcaggaggctgaggtaggagaattgcttgaacctgggaggtggagtttgcagggagccaagatcccaccactgtactccagcctgggcgatagagcaagactcagtctcaaaaaaataaataaaataaaataaagaataggtgagataggaaaaaacagaggaggaatcaagaattcaagactgagcatctggtagaaaatacaaattgtacttctaaaaaatggacaactactgagtatacgaaaatctggccattagtcaatctgttcattaattcattgaacaaccatttatggtcccctactgtggccaggcaccaagccagtactgcagacccaaagatgattgccagtcggtttcttttctgaatgctcgttgaggtatggtagtgccccatgtcacgcttctgagcagggggctgagagagcagatgtgtgggcctgtctatcatggcagttaccctaggagagacctgtaagctgaactgcagtgggggcactttctcccagaggacaggcaggggtgggcgttcaaaaaggctggaggtaagtctcaaagaatctactatcgaataaatcctcctgtgagcttgcttcttagctttggctcaccatttccccagtagcaacatggaaggaacatctccatctgcactgcggcaggttgtagtgggaaacatgcccacctagaatcactgaccttccgtgttaatgtctttgctctgcctgagccaaagcctggcagaagtccggatgggttatcccagggctgaggcgggagccaggtaagcaatagagggcagttttagctacattccaggccgcgctgcagttggcgctttgtgtccattgtggcaagtggtatctgtcctcagggaagaaggtggcaggctaactgtgctctccatccatcctgcaggtggaaggggcatttgtccagggccttggcctcttcaccctagaggagctacactattcccccgaggggagcctgcacacccgtggccctagcacctacaagatcccggcatttggcagcatccccattgagttcagggtgtccctgctccgcgactgccccaacaagaaggccatctatgcatcgaaggtaccattgctctggctctgtgctgtctcttctgatctgccctgggggaggccccagcgggtgatgagaggcctcagtgacccacctggtgggacttcaaagggcatatcttgaggagggaagatggtttgacactgaccttggtaggggcagtgggatagttgggcagtcctgcttgagaaccagttaaattgggttgaggagagcaccttagtatggtctgaagcccacttctttgggtgaatttagagggattccttcatcaaatcttctcatttaaatcctggcccacacccatatctcaggcgtacgtcactggtgcttgatcctaagagatgattttacaggtacagcattagtatggtccaatgacaatcagcacattcactgccaatgggatagcacctttgtacacacccttttacctccttcagttgccaatactcgaccttaagacttgatcaggctgggtgcggtggctcatgcctgtaatcctagcactttgggaggcctaggtgggtggattgcctgagctcagaagttcgagaccagcctgggcaacatggcaaaaccccatctctactaaaaataaaaaaaattagctgggcatggtgggacatgcctgtaatcccagctactcaggaggcttaggcacgagaatcttttgaacctgggaggcagaggctgcagtgagccgtgactggaccattgcaccccagcctgggcgacagagcgagactctgtctcaaaaaaaaaaaaaaaaaaaaaaaaaaagacttggtcagatgcctggaggattgtgtcttctatttcttccttatctgcagtaagtatgaaaaaatggttgaaaattgatttagctgatgcatataatcctagtttgaagaaggaaggcaactaatccttggtaaccaaagaaacatccatttgctcatctcgtggtttactttaccatagtgggtggggaagctgggacatattgtgacccttgccattgactgtcatactttaccatgcttgggaattactgaggcccatgataaaaatgtgcctttctggggcccacgcctagaaattttgctccaataggtgtggcttagggccctggaacatgcattttcagcaagtatctttagtgattctgatataggtggtctctgatctatactttgagggggagaaaaaccaacctatgctagaaaatgttttaattcctgtttgttcttatgcctataattcctgcggatgctgtaatatgactatggtgcctgtaaatgttggtgtcagagttgcaggtagactcttcccctcggctagtgactgaggaaaaggttaaaactcacgtcagattttgtggtctgtaaaatacaattaccgagtataactttaaatggtacattatatgtgcatgatgtcatcagagaaaattacatgagtgttcaaagaaatatgtccatgcggtccagtcatgtactagcttgattgttcttagcccctgatcatttctgactgtgccttcctgctgtatttggtgtggctcccatcagtgctttcctggtgccaccagccctcctcgtgcagagtttgctgcctggcctcctaggtccacttgatgatgtggaggcaggaccacctgcctgagcccctgccctgtgccctgtctgtgttcaggctgttggagagccgcccctcttcctggctgcttctatcttctttgccatcaaagatgccatccgtgcagctcgagctcagcacacaggtaataacgtgaaggaactcttccggctagacagccctgccaccccggagaagatccgcaatgcctgcgtggacaagttcaccaccctggtatgatgcccctcaagcatccactcagtccaggggaggccagggataggctggcaaactgcagggaaggttctgcacagaggagaggtgttgggctctctcctaaccgggcagccttgcatcaatggccacccttcattgtgcatggtatgaagcacacactcgaacttgcaagagaataatgagaaacctccttaaagctatagcttgtagctgggcctccttagagatgggaggaatgtgtggtgcacagtcataactcacagattttattattttccatctagataaggatgatggatgaaactccagagagcctaatctttagtatgagtcagtcgctagcatttttttttttaatttatgttaaggacactgtatgaatattggggggatggagggaaaaagaggaaataattaattagatcaaccacatttgtcctgtgatgatcatgtgccattctgatactggggacacatagatgtccttgaggactttgcaaccttgggaaaccagatgattccattatagggtgattgattactgccttggaggtgtatgtggcagaaagtagtagaagaattccgtagtcattggaatcactttaagaagcacctatgtgatacttgtagttggcaatgcctaacaaaagaagactttgctaggacggttgcttccgctttggggatctcagaaccagcccagcctctgagtggtgctgaagtgggctctgtacagtggctttaattgcctttgcaactgcaacctgtgtggagttagcaggactgggttcttcctgatactcaggtggggctttctctttttaaaattcgatatgctttttggatttataggtaaagtgggtacatgtggtcccaagtggggagggcacagaggaaccccaggggcagacaaatccaccatccgcagacctcaacacagtgggtgaaggaggcatatgggtccccattcaggtcttccttgggtctaccaagagctttagctatggagtgagaactacacagaccgtgtggcactcccaggcagtaaagatgcccacatctgtgcagcaacctacccacagcccagaaccttcccactatggctcccaatgctgacaatcacccaaacccatttccctgagaggagtgggtggcatggctcccaataagctgccacagtttgcaaggcgagctctttgccaacagtctgatgcagaactcagtcattttcgtaagctctctgtggccccagacctgaaacaaagatccgtttctatgttacaacataccagcgatttaaaaattcttgccatggatttgcatgagcagctgattctatgaactttttaaggactgtgagtttcaaaagggatcacatctgtattgtttgtcaccatatctccaaaacctaatagaacgcctgccatgtggaaagtgctcagtgaaaataaatgaatggcttaggaaattaaggagtcctgccctgaggcattctcaacagctaggcctgcagagtggtgcttggccttggaatgatggttggcagtggtgggaggtgaggtatgcaagagagtgggaggagcatgatcagcagaccctggcaatgagaacactatcattccttcctagtgtgtcactggtgtcccagaaaactgcaaaccctggtctgtgagggtctaaagagagagtcctcagcagagtcttcttgtgctgcctttgggcttccatggagcaggaggaacataccacagaacatggatctattaaagtcacagaatgacagacctgtgatttgtcaagatgggatttggaagacaagtgaatgcaatggaagattttgatcaaaaatgtaatttgtaaacacaatgataagcaaattcaaaactgttatgcctaaatggtgaatatgcaattaggatcattttctgtctgttttaatcatgtatctggaatagggtcgggaagggtttgtgctattccccacttactggacagcctgtataacctcaagttctgatggtgtctgtcctttgaagaggattcccacaaacctctagaagcttaaaccgaagttactttaaatcgtgtgccttcctgtgaaagcctggccttcaaaccaatgaacagcaaagcataaccttgaatctatactcaaattttgcaatgaggcagtggggtaaggttaaatcctctaaccatctttgaatcattggaaagaataaagaatgaaacaaattcaaggttaattggatctgattttgtgaagctgcataaagcaagattactctataatacaaaaatccaaccaactcaattattgagcacgtacaatgttctagatttctttcccttcctctttgaagagaatatttgtattccaaatactctttgagtatttacaaaaaagattatgtttaatctttacatttgaagccaaagtaatttccacctagaaatgatgctatcagtcctggcatggtggctcacccctataatcccagcactttgggaggctaaggcaggagaattgcttgagcccagcagtttgagaccagcctgggcaacatagagagctcctgtctttaaaaaaaatttttttaattagttggtcttgatagtgcatgcctgtagtcccaactacttgaaaggctgaggtggagagatcatttgagctcaggaggttgaggctgcagtgagctatgattgcgccactgcactcctgcctgagcgactgagcaagatcttgtctctgaagaaaaaaaaagaaataaaaatgctgctatcaaaatcaagcccaaccagaggtagaagagccaagaagcctgggttctcatcctagctctgtctcttctgtctctatctttgtgatcttggactgtcaattccccttcctgtgatccattttactgcaaacataagggttgcagtaaagggttgtctcacgtcttctgctttaaaagcctataaatatatgacctgaaaactccagttacataaaggatctgcagctatctaaggcttggttttcttactgtcatatgatacctgggtctaatgaactctgctgagatcacctcaagtttctgcggttggtaaagagaacaagggaagaacaaacatcccttttattgctccaaatggtgatttaatccctacatggtgctgggtggacaatgtgtcactgtcacatgccttcactgtataaatccaaccttctgccagagagaatctgtggttctggccatggagggaggatagtggaaatgatatagttggactggtgcttgatgtcactaataaatgaaactgtcagctggttggcgtgtgaaaaatgcttattaattatcatcacaccaaatgctagtatcttcagtatcttggccagccttcatttcttgaaaacacacacacacacacacacacacactttcttacaaaaatccctctccagagtatcctagatatgctggctgttgtacagcatggtcaaggtagaagagctttaaaaactaaacgagtgtttacactgagatccaggtccatttcagatgattttctgggatgttctgggacagggcctgagacctgtcacttgtttttagtgaagctcttggggtgaagcagtgacacagaattcccttgtgtgcttaggtaccaaggtgagagtagagtggtcatgaaaatccaacaagaaagtgctttgtgaactagaaatcactgtgctattcagaggcatcgtccgtctgagacaccttgccttgccctgttgtcttaaagtgcttccagtctatacagtgagatgtcttggtcttttttggtgttgcacaaggaatgcagtcaacactgcctgcggtatcgcctggaacctgcagagggcagtagaagattgaacaaggggcaatattgtccaggtggcccagcagcatcgcatttgttaaccattaggtactgatttaggtaggatctctgcatagaccagatgtcctcggaggcttccccaagagctgaattcaaagctgacttagggttttagaatgacaaaagtctcaaaggagctctccaaagacatgggtcagccgtgggagtctgagctccagatgaatggctagcaaggggaaggcaattcttattttcagagtatctgcaacttgccaggtgacaaggtgaatgctttacctacagggtctcatttaatcctcccagtggttttacgaggtaggtcctttaacccctataaccacatgaaggagaagccctacccctgaccatgctgagggcttactcagagacatttactcctaagtgcccaccacaaccagcgggcagctcccttacaggccaggaaacctggtgtgtattgggagtcacctgcctgtcagagtggacagttgctagccaggctcacggaggtggatggaggcaccaggatgcccaccgtttcatttataggcaggctgaggattctcttgctggcacggccagggtactgagttgtcatcagggcagctgatgagactccccttactccagagctaggtggctgggaggctgggacaggagggcaggacagaggttgtaggaggaactctgggcaagggataaggacagaaatagtcttagctgattgatttatgactgagaacatccccgaacccatggtatttttatcttttgcggagggggagcctggaaacctgtccatttgctgcctattttggatcgtgggacaagatgattcctgacatacagcttccttaaacatgaggtcaccctggtgccccccaggcccaggagcctgcccttttgttttgtttctatcctgaactgggggagtacgtgttcctaaggcaggagacagatcctgtgacaatcttgtcacctgaaacaaatcaacctattgatggaagtggaaatgtcaggcactgcccaaagggattctggcgtcatctctgtccttgaactgtcctgctctggcttgcacaaccccccaggccacctcagcatcccagaactggaaagaccatcaagttcaatctctctattttagagaggagaaacataagcccacagagagaaactgactcgctcagggtcctacggccctttagtagcacagtcagactgacagcaaggctgcctggttcctggtccaggctttttaaaacttaaccatacaccactagtcaactgagccttctattaggaaacctgtgggtcagggagctggggtatatgacagaaatctagttacactgaatgggaagaagacagagcaagcttgaggcccaagataaacactggtgaggagggtaatatgcctgtatgcctatatatgcctatctgcctgtgctcctgtcacaggatgctaaaatgggattctttccgcagccccacagatgggaaacaaaacaatgagtgcaaaaacatcccctcatttcaaagagagatgctgaatctaaatcatctgtgcaagacttgcggagagatggctggaaactaattttgccaccattaactcctcatctacctcaagagtttaaaagattgaagtacagaatttgagagattgggaagagaggtttcctttggaggtagcaaaccatgcattctccctgccaaacaggagagagtgtgtgtgtgcatgtgtgtgtgagaaaaggggggtctcgtggtaaatgagatctgcttgggaagctgtggttggattagactgaggcattaaggtagtgagaggaaggggatgagagccacagggcaaggataccagtgtggccacactggagggatccaccacccaggaggacttcctgaagggtgtcatggaaccccaacaaacaacgggaccatctgatgccccaggatcaaaatagcctcgggctggcttgttccagtgtggcccatgaggagtgcccacacatccttgccctggatcctttgttccagtttaaatttccccctctccccacacctttcagtagggaagaaggtgagcccaagagtgagacccttttctttgagccctgagtgttagtgggagggagcttaggaatggatacacaggctgcaaacattgcactgatgagcccgtgcatcagccctgggagcatagagtctgagtggcatgtcatttgctcccctcctcctatctttccgtctccatcacatcctggcagatttgatcccctgttcagccaattgcccttatctgtgggctgacaacaaagtcgttctggtccatgcatgtgctctctgttcttcctctcactagatggagtattcccaggggtcctcgtggtggctgacgttcttgtgggacactcactacctttccttgcttcccaccgaggctagactgagcacccgccccagggtggtgacacacagcatgcctctgcccactcttgggtgttaggagctgagcataaaagcacgtgtggagtgctcaagctcctctgcttctcctctgcttccctcttccctcttgggcaggttgcttagctcctctgacctcaatctgttcatctgtaaaataggtgtggataatcatatccacctcactgggctgttgtgaagattgaaagaatcagtggggttaaaagcactttttggagtacagagttgttgtcatttgcaggcctttgaccatgccttaaaaggggaagagcaccgggaaatcatgagtaagctttccaaaacttggatggaagggaagatgcaggaatgcatctcgaccataatgaggtgtagggtgatgggtacatggattagctgtgatcttaggccaggtaagagaagctcacctgctccttttggcacaaatgattgattagcatgtctcagcttctcacgtgaatgtgattttcccagcacgttgggctgggtaggagggccggctctcagctccctatagctgactaagtggcacaggtgagatggaatgaagagctcgtcagcctcacctggctatcttcacaagcctttatcagaagtgcagaagtcactctcttctgttattcttgctgtttagttttgtctgacaatgacaacaactggcatttgtatagcaatttgaaaggcactccccgttgcaatgctttctgtaactcttgcgataaacctatgaggtgtgtattacaatcatacccactttacaaacaaagaaactgaggctcggggagaagatgatggtgcagacaagagcactacctgtgaaatcaactgatgtgtttcagtctttgtctcccacttagctgctgtgtgatctcgggcaaattgctgaaatctttacatttctacttccttgtctgtgaatgagatcatggtggtactttcatggggttgtggagaggatggcatccaaaaatgcagggaaagcacaaaacctagtgcctggcaaggaagcttctactcttatacagcgagagtacacctgaggagtgttaactaataagaaaaccaacactgcacagccgcaaacaaaagagaagatgttttaactggggtcttgggaattgtaatgagcaagcagctaaattgtgtcccatccaggtgaggttgggtaggggcttataaaggtttactgtaagtttacctatctggaaggttctagcagagtctttaattgatgatggctggtcacagcttaggatgtgtctgatgatcaatccagtttggcatagctgtttctccaggaggttaatgatcaggtccaatagaaacagctgagatcaaatgcagctggttttacaatttggcccagtttaatagatcagattccatctgggtgtgtacgtgactggggtccaactcctcatgccttcctggcttttttgatacttctgacataaccatctccatttggattttctttctgacagcaatgactttaacctaagactccagatgccaaagcttgagctctttcaatggggtggtgctggctcttaattttttgcattacacacttcagttggcttctagccctggagagaacacacacacactcacacacccatcacaaccatctctctacctgttcactctcttccatccctccatcttggtctcagaatccaggggaaaggacatgcctggctcattgtcagtagaaaagcacctgccttaggaatcagactccctgcttctctttccaaacccagaacagtcacttcagagtgggaggcagctccagaaggcgagggggttgggaggcattgttgataatggcccaaaggtcatttcctccacaccccagggagtcctgattgttgcttaatgataagcctttcccagggtacagaatgtatggcagaatgtatggggtcttggaccctttctccatcctcctgagagccatcagtcttatcaagtgaaagtattctttgtcttcttggagtgggccaagggcttcctgccaggaccctgtgtactttataggcctttgtggattggtgacaatggtcccgtggtggttctgtagtagtgtggagctctgggctctcctatgcactactttaatctggaaacaccccccattctccatccccactgctgggggccaggagtgctgtgcctctgggagatgtggctgtagtaaagttgttggtaatttgtggccttcagctgatgaggagattggcttgcaaggcactcggtatgcatgttcctaagtcccaaaatcaggttttctccttcctcaactatatatgccatgtgttccttttaaaaggccagatgctgggctaagctaggggctcaagtgtaaagagcaagggctgccagcaaacgctctactcagctccagtattttcataagttctcttctcttgggaacattactcaccctctgagcctcagtttcctcatttgtaagtggaggagaataagacctgttgtcagtgtggtgaagttaaatgacatagtcaatgaaaggcacccagcacagcctgggcaacatagtgagacctcgtctctaaaaaaatttttaattagccgtgttcggtggtgtgcacctgtagtcctagctccttgggatgctgaggcgggaggatttcttgagcccaggtgtttgaggctgcagtgcacctcaaatcagaccactgcacttcagcctaggtaacagagagcgactgtctcaaaaggaagaaagaaaataaaagaaaacaaagacactcagcacagcatctgacacctgttgttaggtgcttaataaatgtcaataccttgctgttccaagattctccaagcctatgtacaacaattttgtaaatttagtgtccaaacatgtttatgctatacacttgtagtagacactattgcaaatccttttccttggctgggagttactagttcctctaaatgttggcagctgacagtacacatctgcctattatcttgagaattgccctcagctaaaacagaagtcccctcatctgggagtttataccatcttttaaggctttgcttcttgggaaccaccctatgacaggatatataccattttctacttgcataacttactaacaaatcacattcttccctccctactctctgggtattgtatggctgggaatcctcactctgctataatgtaatatgtggctgggaattcccaatctattgggagctgagacccagtgtagtgaggtaaagtgtgtgtgcttggttgatcttgacaatacgttcaatttctccggagcctcaatcattttccatagccacttgccccattctcacagtagataacatccctggtcaaagcattatcagtttcctctattgctccatcaaatgctcctcttttcttgagatagcaattgagtagccacacacatgacctgagctgtctgcctggccattgttggctcttcatactttgtttacctaagctctgagaaaactatgtgtattgctcaaaggattgcataacattttagagttggaagggacaatccccagtggtcctcaactgggggtgattttacctttagggaacattaggcaatgtctggaaatctctttggttgctgcaactcagggaggggtggtggctcctggaatcaggcaggtagagtgcagggatgccgctaaacatcttatattgcacagggcagcccactgcaacaaagaattattcagcccaaaatgtcagtagaactgaggtggagaaagctgagtctagcctaccacctggatggtggggaggaggcaacggaggattctggaggtggctgtggcagaattaaaagccaagtctccttagctattctttctactgatttagttgtttcattacgataacgtcggtcctttgtagtaagtgtgcgatagcaatctctctacaaaatagaaattttctagcgcaagaacgtaccaagaaattcttatttgtaaatggacactcatacggtatccattccagcatagtgaattaccctccctgcaaaagaatcataaatgatgatatttaactttgatatttgcttctaatcccttatcagagcacataattatgtgttttgtttgaactatagttccttaacatcgttctatgtattggggaaattatacccaggagtggactatctggtttgttagtcatgtttcaaatgtgtttacaaccaggtacatttgtgaatttgcttcttccttgtggagggtctatttttgcctttaaaaacatttttcttaaataaaaagaaaattacagtatgaaccaaacataagtaatatagagaacgataaaatacacatatatacttcccacctggttctaaaaaaatgtttgtttgcccatatttgtttcccatggtaaaagattaatgttacaagtacagttgaagcttcccattttatcaccccactctgagattttgccctctgccttgaggtaattaccatcctaaggttgatgtaaaacattcccatccatgtttttatattttaatgcatgtgaacatatcctaagcagtgcacagcgtatgtgttttaaaattcacacccattaaccaccacaatgactaacattaaaaagactaaaaatatcaagtgtgaataagaggtgaaggaactggaactctcacacactgctaaggtagtaaaatggtacacccaatttggagaactgtttatgagtctctactaaagctgaaagtatgcataccctctgacccagcaattgctgagcatatacccaagcgaaatgcttgcacatatgaaccagaagaaaagtccaagaccgatcatagaagcactgcttgtaatagtcccccaaaattcccaaatgtagatccccaacagagttcataaattaattgcagcatatccatacaatagaataccagcataccaaagaaaataaataaaatccagcttcatacttccattaggatgcatctcacaaacataatatggaattaaagaagccagacacagctggacatggtggcttgcacctgcagtcccagcactttgggaggctgaggcaagaggattgcttaagcccaggagtttgagacccacctgggcgacagggcgagaccccatttctagaaatagaattatttttatatttttattattattatttttgagagagggtctccctctattgcccaggctggagtgcaatggcatgatctcagctaactgcaacctctgcctcctgggttcaagcaattctcatgcctcagcctccccagtagctggaattacagccatgtgccaccaagcctggctaattattgtatttttagtagagacagagttttgccatgttgccaggctggtcttgaactcctggccttggcctcctaaggtgctgggattacaggcgtgagccaccatgcccagctgaaaaaaaaaaaatttgttttaattagccaggcatggtgtcacccacttgaggtcacaattatttgggaggctgaagtgggaggattgcttgagcccgggacgtagaggctgcagtgagccatggtcatgccacttcactccagcctgggcctgggtgacagagggagatccatctcaaaacaaaaacaaaaacccagacgcaaagagttcacactgtatgatcacatatatatacatttcaaaaacaggcaaaaccaacctctagggttagaagtttgatcgtggttacctttggaggtatgtagtactcggggagagcctctgggagggcttggtcatgttctatttatgtgagtgcaggttaccaggccaggtcacttttcaataattcattgaactgttcattatgatttgtacattttaggtaagtatgtatgtatatatatcattcttcaataaaaagtttcctttggaaaagatctacattcatgatatcatattgtgatagccttctgcaattccttgtttctactcaatgttatgcttctaatatttattcatctgggcccatttgatctggtttagccatttatccattacatggtgctttattacatgccaacattaatttttttatccccataccattggatgtttatgctgtttatcatcttcccttttcaatgtagcgaacaattttgaacatgcctccttctgcaatgcatgagagctttcctctaaggcacatagcccacactggaattttggagcataccttccccatttctttattttcaactgttcgtgtcacgttttattgagtctctaataaataatacacatagtttgtgtatggtttggcctcttatgaatagcatgcatgatggacgtgtgtgtttaaatgcaatctaaggatatttctcttttaactggcaaaacaatgtgtttacattaattgtgattaatagcctattttgacatttctatcatctcatttggtgtgtcactactgaccatgttctttctctttttctcattgtctgctgttggcttaatcaagcatccctgtcccccgacccttcccctgccccaccattctccccacctcattctttatatgagtttcgtgcttctacatcctatttctattcttttagaagtcatctctgtatttttagcatgcacacttgattcagcaaagcctatgtttaattaatatttctatgttgtcaccaaacaggatgagaagcttagaatgcattaattgattttcccctctcttcctccgtgtggctataatcaagtgtttcagtttcactttaacactctgattagtcattgttgttattcttgtacaggcaaatatttagttttactcatacgcttacctttgtttttctcgttattgctttcttattccactccctccttctggcttcaatttccttcttttagaaacacacgctttcatagtttttcagccagagtttgtgaatggcaagctctgtctctttttgttaaaaaaaaaaaaggctttattttgccctcattcataaatgatgattttgctgaattagctgctattgatttagctgagtagttatatttcccaaacccttggaatgtatttttctgatgccagcgtaattgtctttcttttgcaggcactatctcttctctggctgtttttcttctgttaaagtattctattttaaggtgttctgttgtctaggcatggattaactttttacctattattcttggaattcaccatgcttctttatcctgagaacttgttttcaagtctggaaaattctaagcccttatttattgcaacattatttctcctccattctctctatcctttctttccttaacttctaaatagatttgtactgaaacttctaattctatcctccatgtatctttaggcctcatatttctcatctctatctctgtggtgctttctgggtaatttcctaggtctacatttcaattatttaatgatctcttcaactctgtctattttcatgtttaagtaaatcgagtttttagtcttagtttctttacttttattttcatacattctgattttttttcacattcacttgagttagtacagactaggttatgctacagaaagaaaataatcccaaatttcccaatctccatgattgaatacagcaatagtttatttctcactcctttgtacccagtggattttggtggggtctgttctacacggtcactcagagaccaaggacgaaagaggctccatcatctagcagttgcactacctggaatatgaggcttactgggtgtctgcaacagggaaacggacatctggaggatgtcacattagctatcctatggccaatattggaattaacacgcagcaccacctgccacagaccactggccagaacaagtcatgtagctctaccaagtgccagtgcatggggaattttggtagtgcttcgtatgtgatgatcgacactatttccttttccatttattgtattttatcattgtgttctactccttctttcatctctttaaaacaagtaaaatgcttagaacaatgcttaccatgtagtaagaacttcacagaagtgttttctaacattttaagtacaatttatttttaacagattttacaagttctcatagtctaattctttccttcaatgtggaatatttattcacatagatcatttttttttcattataagcttattctcaatgagtgttccgtttcctgtggtgaacttgtgctctgtgggttggggaacgattccctatagagcacatgtgtcttctgtgcttttattgcctttttatcagttcaaaggtgagcttctaccatgtgtgctgggtaaatatagaccccacacctggaattttgatttttcatataaactttttgttttcttttcttcccaagaactctgagtagatacaatgctttcttgctgcttccacaggctaatgaatagttttttctctcccctcttcatgggtactctacatagggtgctttccaacactacaaccgattctctgattctctggacaccagagtgtccaacagttcagatcaattctgacactaactacctggagttatcacagacctcacagcctaatggctcagtctcataagactgcccctgcttcagtgccaggcctcctgtacttctggccaactggctataaataggggttcccatgcctctctcctcatgtttgatagtttgccaggacagctcacagaactcaggaatgcactttacttacattgaccagcttattataaaggatgcaaatgagcaaccagatgagttccaggcatatagggaacataggaaaaggtctgaagggtcctaagtgcaggagcttctgttctatggagttgaggtgcccccatctcctggcacatggatgtgttcaacccggaagctccctgaaccccatcgtttagaggttttttatggaggttggatcacataggcacactctattattaa

(19) United States
(12) Patent Application Publication
(10) Pub. No.: US 2017/0101678 A1
(43) Pub. Date: Apr. 13, 2017

(54) METHOD FOR SCREENING RISK OF DRUG-INDUCED TOXICITY

(71) Applicant: RESEARCH CENTER FOR BIOTECHNOLOGY AND MEDICINE POLICY, Taipei City, Taiwan (CN)

(72) Inventors: Oliver Yao-Pu HU, Taipei City (TW); Cheng-Huei HSIONG, Taipei City (TW); Hsin-Tien HO, Taipei City (TW); Tung-Yuan SHIH, Taipei City (TW)

(73) Assignee: RESEARCH CENTER FOR BIOTECHNOLOGY AND MEDICINE POLICY, Taipei City (TW)

(21) Appl. No.: 15/114,580

(22) PCT Filed: Jan. 27, 2014
(86) PCT No.: PCT/CN2014/071598

§ 371 Date: Jul. 27, 2016

(51) Int. Cl. C12Q 1/68

(52) U.S. Cl. CPC C12Q 1/6883; C12Q 2600/156; C12Q 2600/106; C12Q 2600/142

(57) ABSTRACT

Present invention provides a method for screening drug-induced toxicity and screening for drug-induced toxicity by using the NAT2, CYP2E1 and Xanthine Oxidase genes, in particular, provides a method for screening TB drug-induced hepatotoxicity. The method of present invention includes providing test specimen, detection of at least one type of SNPs of the NAT2, CYP2E1 and Xanthine Oxidase genes from the DNA of said test specimen; presence of the SNP genotype indicates the test specimen has a risk for developing anti-TB drug-induced toxicity; said SNP genotype is selected from at least one of the following groups or their combinations thereof: rs1041983, rs1112005, rs1495741, rs1799930, rs1799931, rs1801280, rs1961456, rs2087852, rs11996129, rs2031920, rs2249695, rs3813865, rs3813867, rs1884725, rs2295475 and rs17011368. Moreover, the screening method of the invention includes the test specimen, detection of at least one type of SNPs of the Xanthine Oxidase gene from the DNA of said test specimen; presence of the SNP haplotype indicates the test specimen has a risk for developing high uric acid and its related diseases; said SNP genotype is selected from at least one of the following groups or their combinations thereof: rs1884725, rs2295475 and rs17011368.

### BACKGROUND OF THE INVENTION

1. Field of the Invention

Present invention relates to a method for screening the risk of drug-induced toxicity, such as anti-tuberculosis (TB) drug-induced toxicity, in particular, a method for detection of anti-TB drug-induced hepatic injury by using single nucleotide polymorphism (SNP) of NAT2, CYP2E1 and Xanthine Oxidase genes. In addition, the method of present invention includes a test specimen and the Xanthine Oxidase gene of the test specimen contains at least one SNP haplotype, presence of said SNP haplotype indicates the test specimen has a risk of developing high uric acid and its related diseases.

2. Description of the Prior Art

Isoniazid (INH) is the most common drug for treating tuberculosis (TB) and usually administered combine with rifampicin (RMP) or pyrazinamide (PZA). Nonetheless, INH will also cause serious side effect-hepatotoxicity while used for treating tuberculosis. Such side effect is generated by the hepatotoxic metabolites resulted from metabolism of INH in vivo by liver enzymes, N-acetyltransferase 2 (NAT2) and Cytohrome P450 2E1 (CYP2E1). Human body utilizes Glutathione (GSH) and toxic metabolites to generate water-soluble sulfhydryl conjugation to help elimination of such toxic substances. During the process of formation of said conjugation, a specific enzyme Glutathione S-transferases (GST) is required for catalysis of GSH to facilitate binding of GSH to its substrate. Therefore, NAT2, CYP2E1 and GST as well as the INH-induced hepatotoxicity are associated with the detoxification in vivo.

INH usually convert to N-acetylisoniazid through NAT2 in the liver after entry into the body through intestinal absorption. Studies have indicated that the NAT2 gene is polymorphic, thus resulting in a significant variation on the activity of NAT2. Individuals with higher NAT2 activity showed rapid acetylation phenotype that is consequently results in lower serum INH concentrations and limited therapeutic effects. On the other hand, individuals with lower NAT2 activity showed slow acetylation phenotype and an increased serum INH concentration, which leads to toxicity or side effect. The study of Weber et al. suggested that in Caucasians around 40%˜70% of them belong to NAT2 rapid acetylators, whereas in the eastern populations only 16.7˜26.7% of the people belong to rapid acetylators and most importantly, the cause of such difference is genotypic variation between races, which leads to the difference of the NAT2 activity.

By far, 36 NAT2 alleles have been identified and among which NAT2*4 is the wild-type allele and is also the first NAT2 allele found and is classified as a rapid acetylation allele. Other alleles, such as NAT2*11A, *12A, *12B, *12C and *13 also belong to the group of rapid acetylation alleles and this is because such polymorphic allele s usually will not cause variation of amino acids or variation of conservative amino acids. Alternatively, alleles such as NAT2*5, *6 *10, *11B, *12D, *17, *18 and *19 belong to the group of slow acetylation alleles due to variation of non-conservative amino acids caused by these alleles. From the study conducted by Fretland et al., 24 alleles belong to the group of slow acetylation. Among the NAT2 alleles, a total of 16 single nucleotide polymorphisms (SNPs) were found and they are located in the coding region of allele 111, 190, 191, 282, 341, 364, 411, 434, 481, 499, 590, 759, 803, 845, 857 and 859, and these SNP haplotypes are one of the reasons that cause polymorphism of the NAT2 enzyme. For example, mutation of 341 (Ile114→Thr) is the major characteristic of NAT2*5, whereas variations of 191(Arg64→*Gln) and 590 (Arg197→*Gln) are the main features of NAT2*14 and *6. Abe et al. have shown that in Caucasians the frequency of NAT2*5 and NAT2*6 were almost identical to that of the NAT2*4 wild type; in contrary, in eastern populations allele NAT2*5 was rarely found and the frequency of NAT2*4 wild type allele increase instead. Therefore, most Caucasians belong to the group of slow acetylation while eastern populations usually belong to the group of rapid acetylation. The study of Sim and Hickman pointed out that the difference of NAT2*5 between eastern and western population is just the cause of different frequency of the genotype of low acetylation (Caucasians 52-68% and Japanese 10-15%). The product produced by hydrolysis of INH metabolite in the liver by NAT2 is acetylhydrazine which will be further processed into metabolites with hepatotoxicity by CYP2E1. CYP2E1 is an alcohol-induced microsomal enzyme which participates in the metabolism of carcinogens and certain drugs in vivo and is considered correlated to development of alcohol-induced liver disease and certain cancers (such as hepatoma). CYP2E1 has been proven to be polymorphic. (see http://www.cypalleles.ki.se/ for relevant variations). A technology called restriction fragment length polymorphism (RFLP) can be used to analyze different CYP2E1 alleles.

Yi-Shin Huang et al. indicated in the study of gene polymorphisms of CYP2E1 in Taiwanese people that use of the restriction enzyme RsaI in RFLP identified two alleles, CYP2E1 c1 and CYP2E1 c2 (c1 indicates wild-type allele and c2 indicates mutated allele), and the incidence of hepatotoxicity in c1 homozygote with the genotype of CYP2E1 c1/c1 treated with anti-TB drug is higher than CYP2E1 enzyme (CYP2E1 c1/c2 and CYP2E1 c2/c2) of c2 allele (c1/c1: 20.0%, odds ratio=2.52; c2/c2: 9.0%, P=0.009). Moreover, according to the paper published by Nicolas Vuilleumier et al., use of RsaI and TaqI restriction enzymes in RFLP for analysis of the patients treated with INH indicated that among the CYP2E1 alleles, CYP2E1 1A (wild-type allele), CYP2E1 1B and CYP2E1*5, the patients who are CYP2E1 1A homozygotes had a higher risk of developing hepatitis or increased liver readings when compared with patients of other genotypes (1A/1A: 39%, followed by 1A/1B: 15%, P=0.02).

The major metabolic enzyme of PZA, xanthine oxidase (XO), is polymorphic and has significant effect on the enzyme activity. In individuals with higher XO activity have a higher risk developing xanthinuria, whereas in the individuals with lower XO activity, oxidation is slow and thus PZA is accumulated in the body, which leads to in vivo toxicity or side effects. The major cause of such difference is gene polymorphisms. Thus far, studies of polymorphism on PZA-related metabolic enzymes have reported 18 alleles. Ichida's study indicated in 1993 that in addition to single nucleotide polymorphisms (SNPs) of the coding region of allele 691, 783, 2533, 3183 and 3309, which are 691(Arg231→*Gly), 783(Asn261→*Lys), 2533(Ala845→Thr), 3183(Lys1061→Lys) and 3309(Glu1103→Glu), respectively, some nucleotides also showed amino acid polymorphisms but none were found to have an effect on XO activity, which may be due to the highly conserved region of alleles, for example, 783(Asn261→*Lys). In 2008, Kudo studied 21 alleles and found 445(Arg149→*Cys) and 2729(Thr910→Lys), and when compared with the wildtype, the xanthine oxidase of the two SNP haplotypes mentioned above did not have enzymatic activity; in addition, 1663(Pro555→Ser), 1820(Arg607→*Gln), 1868(Thr623→*Ilu), 2727(Asn909→*Lys), 3449(Pro1150→Arg) and 3953(Cys1318→Tyr), 2533(Ala845→Thr), 3183(Lys1061→Lys) and 3309(Glu1103→*Glu), the xanthine oxidase activity of the six SNP haplotypes mentions above were all lower than the wildtype; for 2107(Ile703→*Val) and 3662(His1221→*Arg), the xanthine oxidase activity of these two SNP haplotypes were 2-fold higher than wildtype. In summary, the coding region of the alleles with SNPs are just one of the reasons that cause gene polymorphisms of xanthine oxidase and have an effect on the activity of xanthine oxidase (Tostmann et al., 2010). Around 20% of Caucasians belong to the group with relatively lower activity of xanthine oxidase, whereas roughly 11% of eastern populations have low XO activity, among which the enzyme activity is higher in men than in women and the main reason that causes such difference is activity polymorphisms or lack of the activity of xanthine oxidase caused by gene polymorphisms.

In summary, the activity of the major metabolic enzymes related to anti-TB drug-induced hepatotoxicity, NAT2, CYP2E1 and Xanthine Oxidase, are affected by gene polymorphisms and thus a biomarker that can be used for testing, treating and ameliorating the anti-TB drug-induced hepatotoxicity, is needed for clinical practice.

### SUMMARY OF THE INVENTION

Present invention provides a method for screening the risk of drug-induced toxicity, such as anti-TB drugs, including a test specimen and the SNPs of at least one target gene of the test specimen, wherein said target gene is consisting of NAT2, CYP2E1 and Xanthine Oxidase, and whether the test specimen belongs to the high risk group of developing liver injury is determined based on the genotype of SNPs of the target genes, or alternatively, be used as an indicator for prognosis.

In the examples of the invention, present invention provides a method for screening the risk of drug-induced toxicity, such as anti-TB drugs, including a test specimen and the SNPs of at least one target gene of the test specimen; wherein said target gene is consisting of NAT2, CYP2E1 and Xanthine Oxidase; wherein said target gene NAT2 is the nucleotide sequence indicated in GenBank Accession Number: NC_000008.10 or other sequences have the similarity of 90% when compared with NAT2, the target gene CYP2E1 is the nucleotide sequence indicated in GenBank Accession Number: NC_000010.10 or other sequences have the similarity of 90% when compared with CYP2E1; target gene Xanthine Oxidase is the nucleotide sequence indicated in GenBank Accession Number: NC_000002.11 or other sequences have the similarity of 90% when compared with Xanthine Oxidase; in addition, determine whether the specimen is of the high-risk group of developing liver injury induced by anti-TB drug based on the genotype of the SNPs of the target gene or be used as an indicator for prognosis; wherein the test specimen is blood, amniotic fluid, cerebrospinal fluid, tissue fluid, other body fluids, skin, hair, muscle, placenta, gastrointestinal tract, other organs and tissues and other biological specimen from the body; wherein the drug is an anti-tuberculosis drug and any one or the combination of the two drugs for treating other diseases; wherein the anti-TB drug is any one or the combination of the drugs containing isoniazid, rifampin, pyrazinamide and ethambutol; wherein the toxicity hepatotoxicity and/or increased ALT and AST; wherein the SNP is selected from the following group:

### NAT2:

rs1208

rs1041983

rs1112005

rs1495741

rs1799929

rs1799930

rs1799931

rs1801280

rs1805158

rs1961456

rs2087852

rs4986996

rs4986997

rs11996129

rs72466457

rs72466460

rs72466461

### CYP2E1:

rs2031920

rs2249695

rs3813865

rs3813867

### XO:

rs566362

rs1884725

rs2295475

rs17011368

rs72549369;

The testing method for the SNP genotype of the target gene is consisting of at least one of the following methods: restriction fragment length polymorphism (RFLP), tetra-primer ARMS-PCR), PCR molecular beacons, SNP microarrays, temperature gradient gel electrophoresis and denaturing high performance liquid chromatography.

Wherein the SNP genotype of rs1041983 is TT, TC or CT; the SNP genotype of rs1112005 is TT, TC or CT; the SNP genotype of rs1495741 is AA genotype, the SNP rs1799930 is AA, AG or GA; the SNP genotype of rs1799931 is AA, AG or GA; the genotype of SNP rs1801280 is CC, CT or TC; the genotype of SNP rs1961456 is AA, AG or GA; the SNP genotype of rs2087852 is CC, CT or TC; the SNP genotype of rs11996129 is CC, CT or TC; the SNP genotype of rs2031920 is CC; the SNP genotype of rs2249695 is CC; the SNP genotype of rs3813865 is GG; the SNP genotype of rs3813867 is GG; the SNP genotype of rs1884725 is AA, AG or GA; the genotype of SNP rs2295475 as AA, AG or GA, or the SNP genotype of rs17011368 is TT, indicating the individual has a higher risk of developing drug-induced toxicity.

wherein the SNP genotype of rs1495741 is AA genotype of or the SNP genotype of rs2295475 is AA, indicating the individual has a higher risk of causing toxicity.

The present invention also provides a method for screening high uric acid and its related diseases, wherein the method comprises of the following steps:

Step 1: obtain the test specimen from test subjects, wherein the test specimen are mammalian subjects;

Step 2: examine the SNP of Xanthine Oxidase of the genomic DNA of the test specimen, wherein the Xanthine Oxidase gene is the nucleotide sequence indicated in GenBank Accession Number: NC_000002.11 or other sequences have the similarity of 90% when compared with Xanthine Oxidase;

And Step 3: according to the genotype of the SNP of the Xanthine Oxidase gene, determine whether the sample is of the high-risk group of high uric acid and its related diseases or be used as a treatment indicator or prognostic indicator.

Wherein the test specimen is blood, amniotic fluid, cerebrospinal fluid, organ, tissue, cell, lymphatic fluid, tissue fluid, other body fluids, skin, hair, muscle, placenta, and gastrointestinal tract and oral mucosa.

Wherein the SNP is at least one of rs1884725 and rs2295475; wherein the SNP rs1884725 as GA, AG or AA genotype and the SNP rs2295475 as GA, AG or AA genotype, indicating the individual has an increased risk of developing high uric acid and its related diseases.

### BRIEF DESCRIPTION OF THE DRAWINGS

### DETAILED DESCRIPTION OF THE PREFERRED EMBODIMENT

The following examples will further illustrate the present invention, however these examples are merely illustrative purposes, and should not be construed as limiting embodiment of the present invention.

### EXAMPLE 1

### Relevant Studies of NAT2, CYP2E1,and GSTM1 SNPs- and Anti-TB Drug-Induced Hepatotoxicity in Taiwan

Analysis of the correlation between SNP and TB drug-induced hepatotoxicity based on the genotype test results obtained from more than 300 patients with tuberculosis and clinical data, we further identified 7 NAT2 SNPs which showed significant correlation with TB drug-induced hepatotoxicity by using 2×2 and 2×3 Chi square test, which 2 additional SNPs that showed significant correlation. The result indicated if the TB patients carry any one of the 7 NAT2 SNPs, their risk of developing TB drug-induced hepatotoxicity was 1.8-fold to 10.3 fold higher than those do not carry any of the 7 SNPs.

These NAT2 SNPs are rs1779931 (homozygous, odd ratio=10.294, p=0.009), rs1799930 (heterozygous+homozygous, odd ratio=1.824, p=0.042), rs11996129 (heterozygous+homozygous, odd ratio=1.897, p=0.030), rs1961456 (homozygous, odd ratio=3.333, p=0.004), rs1112005 (heterozygous+homozygous, odd ratio=1.824, p=0.042), rs1041983 (homozygous, odd ratio=2.175, p=0.047) and rs2087852 (hetero+homozygous, odd ratio=2.076, p=0.014). The correlation between CYP2E1 SNP and anti-TB drug-induced hepatotoxicity or GSTM1 SNP and anti-TB drug-induced hepatotoxicity are not significant (Table 1 and Table 2).

TABLE 1
Risk analysis of NAT2 SNP genotypes and anti-TB drug-
induced hepatotoxicity

| | Hepatotoxicity | | | |
|---|---|---|---|---|
| | Absent | Present | | |
| SNP ID | (n = 252) | (n = 56) | Odds Ratio | p-value |
| rs1799931 | | | | |
| GG | 175 | 34 | Reference | |
| GA | 75 | 18 | 1.235 | 0.512 |
| AA | 2 | 4 | 10.294 | 0.009 |
| GA + AA | 77 | 22 | 1.471 | 0.206 |
| rs179930 | | | | |
| GG | 150 | 25 | Reference | |
| GA | 87 | 25 | 1.724 | 0.082 |
| AA | 15 | 6 | 2.400 | 0.098 |
| GA + AA | 102 | 31 | 1.824 | 0.042 |
| rs11996129 | | | | |
| TT | 148 | 24 | Reference | |
| TC | 87 | 25 | 1.772 | 0.070 |
| CC | 17 | 7 | 2.539 | 0.062 |
| TC + CC | 104 | 32 | 1.897 | 0.030 |
| rs1961456 | | | | |
| GG | 120 | 18 | Reference | |
| GA | 104 | 24 | 1.538 | 0.204 |
| AA | 28 | 14 | 3.333 | 0.004 |
| GA + AA | 132 | 38 | 1.919 | 0.035 |
| rs1112005 | | | | |
| CC | 150 | 25 | Reference | |
| CT | 85 | 23 | 1.624 | 0.129 |
| TT | 17 | 7 | 2.471 | 0.070 |
| CT + TT | 102 | 31 | 1.824 | 0.042 |
| rs1041983 | | | | |
| CC | 79 | 15 | Reference | |
| CT | 126 | 22 | 0.920 | 0.818 |
| TT | 46 | 19 | 2.175 | 0.047 |
| CT + TT | 172 | 41 | 1.255 | 0.491 |
| rs2087852 | | | | |
| TT | 149 | 23 | Reference | |
| TC | 86 | 26 | 1.959 | 0.034 |
| CC | 17 | 7 | 2.668 | 0.051 |
| TC + CC | 105 | 25 | 2.076 | 0.014 |

Note:
The heterozygous alleles shown in the table are not in order. For example, there is no difference between the GA and AG genotype of rs1799931 and both are presented in GA, same for other SNPs.

TABLE 2
Risk analysis of CYP2E1 and GSTM1 SNP genotypes and TB drug-
induced hepatotoxicity

| | Hepatotoxicity | | | |
|---|---|---|---|---|
| | Absent | Present | | |
| SNP ID | (n = 252) | (n = 56) | Odds Ratio | p-value |
| CYP2E1 | | | | |
| rs2249695 | | | | |
| CC | 90 | 23 | Reference | |
| CT | 122 | 21 | 0.674 | 0.234 |
| TT | 37 | 12 | 1.269 | 0.557 |
| CT + TT | 162 | 33 | 0.797 (0.818) | 0.452 (0.297) |
| rs2031921 | | | | |
| TT | 153 | 35 | Reference | |
| TC | 78 | 17 | 0.953 | 0.882 |
| CC | 12 | 4 | 1.457 | 0.535 |
| TC + CC | 90 | 21 | 1.020 (1.038) | 0.948 (0.507) |
| rs2070676 | | | | |
| CC | 160 | 33 | Reference | |
| GC | 87 | 21 | 1.170 | 0.611 |
| GG | 4 | 1 | 1.212 | 0.865 |
| CG + GG | 91 | 22 | 1.172(1.223) | 0.602(0.301) |
| rs2515641 | | | | |
| CC | 160 | 33 | Reference | |
| TC | 85 | 21 | 1.198 | 0.560 |
| TT | 4 | 1 | 1.212 | 0.865 |
| CT + TT | 89 | 22 | 1.199 (1.229) | 0.553 (0.296) |
| rs3813865 | | | | |
| GG | 161 | 33 | Reference | |
| GC | 85 | 21 | 1.205 | 0.546 |
| CC | 6 | 2 | 1.626 | 0.562 |
| GC + CC | 91 | 23 | 1.233 (1.461) | 0.487 (0.13) |
| GSTM1 | | | | |
| rs2071487 | | | | |
| null | 147 | 30 | Reference | |
| T0 or TT | 105 | 25 | 1.167 (1.343) | 0.607 (0.199) |

Note:
The heterozygous alleles shown in the table are not in order. For example, there is no difference between the GA and AG genotype of rs1799931 and both are presented in GA, same for other SNPs.

### EXAMPLE 2

### Evaluation of the Effects of the NAT2 Haplotypes on Hepatic Side Effects by Multi-Point Methods

Perform multi-points analysis on the 7 high-risk NAT2 haplotypes to further evaluate the effect of the combinations on high hepatic side effects. We tried to conduct statistical test on 2 to 4 SNPs haplotypes and have identified at least 8 kind of combinations will significantly increase the risk of hepatotoxicity induced by anti-TB drug, the highest odds ratio up to 4.1 fold (p<0.001) (see Table 3). Among combination between two NAT2 SNPs, patients who carry any two or more points variability of rs1961456 or rs1799931 (both are heterozygous or at least one is homozygous, or both) can be defined as the high-risk group of TB-induced hepatotoxicity, which account for about 26% of the proportion of all patients; in the high-risk group the ratio of patients who developed hepatotoxical side effect was 36% (29 out of 81 cases developed hepatotoxicity), which is 3-fold of the low-risk population prevalence of ratio of 12%, was significantly higher and the risk of these patients was 4.1-fold of the low-risk group (p<0.001). The two SNP combination also has a significant impact for rs1961456 and rs1041983, and the ratio of their high-risk group was 20% and the incidence of hepatotoxicity for the high- and low-risk group was 35% and 14%, respectively, and the odd ratio of the high-risk group for developing hepatotoxicity was 3.26 fold of the low-risk group (p<0.001). With a high-risk portfolio rs1961456 and rs2087852, the proportion of high risk group was 12%, the incidence of hepatotoxicity for the high- and low-risk group was 37% and 16%, respectively, and its odd ratio was 3.17 fold of the low-risk group (p<0.001).

TABLE 3
Risk analysis of NAT2 SNP haplotypes and TB drug-induced hepatotoxicity(I)

| | | Hepatotoxicity | | | |
|---|---|---|---|---|---|
| NAT2 | Risk group | Absent | Present | | |
| haplotype | (% of population) | (% of each group) | (% of each group) | Odds Ratio | p-value |
| Haplotype*4 | Low (71%) | 192 (88%) | 26 (12%) | | |
| (rs1961456 * rs1799931 * rs2087852 * | High (29%) | 60 (67%) | 30 (33%) | 3.692 | <0.001 |
| rs1041983) | | | | | |
| Haplotype*3-1 | Low (73%) | 198 (88%) | 27 (12%) | | |
| (rs1961456 * rs1799931 * rs2087852) | High (27%) | 54 (65%) | 29 (35%) | 3.938 | <0.001 |
| Haplotype*3-2 | Low (71%) | 193 (88%) | 26 (12%) | | |
| (rs1961456 * rs1799931 * rs1041983) | High (29%) | 58 (66%) | 30 (34%) | 3.840 | <0.001 |
| Haplotype*3-3 | Low (79%) | 209 (86%) | 33 (14%) | | |
| (rs1961456 * rs2087852 * rs1041983) | High (21%) | 42 (65%) | 23 (35%) | 3.468 | <0.001 |
| Haplotype*3-4 | Low (79%) | 208 (86%) | 35 (14%) | | |
| (rs1799931 * rs2087852 * rs1041983) | High (21%) | 44 (68%) | 21 (32%) | 2.836 | <0.001 |
| Haplotype*2-1 | Low (74%) | 200 (88%) | 27 (12%) | | |
| (rs1961456 * rs1799931) | High (26%) | 52 (64%) | 29 (36%) | 4.131 | <0.001 |
| Haplotype*2-2 | Low (80%) | 212 (86%) | 35 (14%) | | |
| (rs1961456 * rs1041983) | High (20%) | 39 (65%) | 21 (35%) | 3.262 | <0.001 |
| Haplotype*2-3 | Low (88%) | 228 (84%) | 42 (16%) | | |
| (rs1961456 * rs2087852) | High (12%) | 24 (63%) | 14 (37%) | 3.167 | 0.001 |

Among the arrangement of three NAT2 SNP, we found the high-risk group carrying rs1961456, rs1799931 and rs2087852 had the highest odds ratio (odds ratio=3.938, p<0.001). The proportion in the high-risk group was 27%. The incidence of hepatotoxicity for the high- and low-risk group was 35% and 12%, respectively. Followed combination of rs1961456, rs1799931 and rs1041983, and the percentage of the number of patients in the high-risk group of this combination was 29%, the incidence of hepatotoxicity for the high- and low-risk group was 34% and 12%, respectively, and the odd ratios of developing hepatotoxicity for the high-risk group was 3.84 fold of the low-risk group (p<0.001).

Among the arrangement of 4 NAT2 SNPs, the combination of rs1961456*rs1799931*rs2087852*rs1041983 was most representative the proportion of high-risk group was 29%, the incidence of hepatotoxicity for the high- and low-risk group was 33% and 12%, respectively, and the odd ratio of developing hepatotoxicity for the high-risk group was 3.692 fold of the low-risk group (p<0.001).

Though theoretically 37 combinations may be generated based on different arrangements of the 7 NAT2 high-risk SNP haplotypes, linkage disequilibrium (LD) between the haplotypes may significantly reduce and affect the number of combinations. Moreover, from the results, the combination of 4 SNPs (rs1961456*rs1799931*rs2087852*rs1041983) or 3 SNPs (rs1961456*rs1799931*rs1041983) both showed the highest estimated number of patients in the high-risk group (both were above 29% of the total number of patients) and the highest number of cases that developed hepatotoxicity (30 high-risk cases out of the total 56 cases), but the combination of rs1961456*rs1799931 showed the most significant difference in the incidence between the high- and low-risk groups (12% vs. 36%, odd ratio=4.131), in addition, only gene polymorphisms of two loci are required, which offers competitive advantages in clinical application or development of rapid test chip or reagents, suggesting combinations of these two SNPs are the best representations for predicting the risk of antiTB drug-induced hepatotoxicity and also practicable.

### EXAMPLE 3

### Validation of the Risk of Hepatotoxical Side Effect in TB Patients with the Combinations of High-Risk Haplotypes in a Prospective Trial

Based on the aforementioned research results, we performed clinical follow-ups in a prospective trial and enrolled newly diagnosed TB patients and those patients who just started or re-started the treatment, the number of TB patients enrolled were 61 and the gene analysis of 59 patients had been completed. However, only 55 patients were analyzed after exclusion of those who had hepatitis B or who were hepatitis C carriers or who had more than half of the total SNP no call number, among which 4 cases were determined to have TB drug-induced hepatotoxicity. After haplotype analysis, the subjects can be divided into the high-risk group and the low-risk group and the number of each haplotype as well as the number of patients who developed hepatotoxicity are shown in Table 4.

TABLE 4
Risk analysis of NAT2 SNP haplotypes and TB drug-induced hepatotoxicity (II)

| | Low risk group | | | High risk group | | | | |
|---|---|---|---|---|---|---|---|---|
| | | Hepatotoxicity | | | Hepatotoxicity | | | |
| SNP-SNP combination | N (%) | Absent | Present | N (%) | Absent | Present | Odds ratio | Significance |
| rs1961456 * rs1799931 | 37 (70%) | 36 | 1 | 16 (30%) | 13 | 3 | 8.31 | 0.042 |
| rs1961456 * rs2087852 | 22 (40%) | 21 | 1 | 33 (60%) | 30 | 3 | 2.10 | 0.525 |
| rs1961456 * rs1799930 | 23 (44%) | 22 | 1 | 29 (56%) | 26 | 3 | 2.54 | 0.650 |
| rs1961456 * rs1041983 | 19 (35%) | 18 | 1 | 35 (65%) | 32 | 3 | 1.69 | 0.658 |
| rs1961456 * rs1112005 | 27 (51%) | 26 | 1 | 26 (49%) | 24 | 2 | 2.17 | 0.530 |
| rs1961456 * rs11996129 | 22 (42%) | 21 | 1 | 31 (58%) | 28 | 3 | 2.25 | 0.486 |
| rs1799931 * rs2087852 | 38 (73%) | 37 | 1 | 14 (27%) | 11 | 3 | 10.09 | 0.024 |
| rs1799931 * rs1799930 | 42 (81%) | 40 | 2 | 10 (19%) | 8 | 2 | 5.00 | 0.104 |
| rs1799931 * rs1041983 | 32 (60%) | 31 | 1 | 21 (40%) | 18 | 3 | 5.17 | 0.132 |
| rs1799931 * rs1112005 | 44 (86%) | 42 | 2 | 7 (14%) | 6 | 1 | 3.50 | 0.309 |
| rs1799931 * rs11996129 | 37 (74%) | 36 | 1 | 13 (26%) | 10 | 3 | 10.80 | 0.020 |
| rs2087852 * rs1041983 | 19 (35%) | 18 | 1 | 35 (65%) | 32 | 3 | 1.98 | 0.658 |
| rs2087852 * rs1799930 | 23 (45%) | 22 | 1 | 28 (55%) | 25 | 3 | 2.64 | 0.708 |
| rs2087852 * rs1112005 | 30 (55%) | 29 | 1 | 25 (45%) | 23 | 2 | 2.52 | 0.448 |
| rs2087852 * rs11996129 | 22 (42%) | 21 | 1 | 31 (58%) | 28 | 3 | 2.25 | 0.486 |
| rs1041983 * rs1799930 | 20 (38%) | 19 | 1 | 32 (62%) | 29 | 3 | 1.97 | 0.565 |
| rs1041983 * rs1112005 | 21 (40%) | 20 | 1 | 31 (60%) | 29 | 2 | 1.38 | 0.798 |
| rs1041983 * rs11996129 | 20 (38%) | 19 | 1 | 32 (62%) | 29 | 3 | 1.97 | 0.565 |
| rs1799930 * rs1112005 | 27 (54%) | 26 | 1 | 23 (46%) | 21 | 2 | 2.48 | 0.459 |
| rs1799930 * rs11996129 | 22 (45%) | 21 | 1 | 26 (53%) | 24 | 2 | 2.63 | 0.404 |
| rs1112005 * rs11996129 | 29 (57%) | 28 | 1 | 22 (43%) | 20 | 2 | 2.8 | 0.396 |

Based on the results obtained by far, the combination of SNP rs1961456*rs1799931 is quite representative in predicting TB-drug induced hepatotoxicity for the high-risk TB patients. After haplotype analysis, 16 cases of the high-risk group of rs1961456*rs1799931 were found among the subjects who were subjected to analysis, accounting for 30% of the total enrolled number of subjects and among which 4 cases were determined to be the patients with TB drug-induced hepatotoxicity, 3 cases were in the high-risk group of rs1961456*rs1799931 and the incidence of hepatotoxicity for the high-risk group was 18.8% ( 3/13), which is significantly higher than 2.7% ( 1/37) of the low-risk group; its risk ratio was 8.31 fold (p<0.05) of the low-risk group. The results of this prospective trial confirmed the results of past retrospective analysis and indicated if the TB patients carry the combination of high-risk rs1961456*rs1799931 haplotypes, their risk of developing hepatotoxicity increased significantly, 8.3 fold higher than the low-risk group and the ratio of patients developing hepatotoxicity increased from 2.7% for the low-risk group to 18.8%. According to the analyses conducted in the past, the number of TB patients who belong to this high-risk group is roughly 30% of the total number of patients. In addition to the combination of rs1961456*rs1799931, from the results of this prospective trial, we also found the high-risk combinations of rs1799931*rs2087852 or rs1799931*rs11996129 will significantly increase the risk of hepatotoxicity for more than 10 fold (p<0.05), which is a combination that was not found in the past analysis. Among which, 14 cases carry the high-risk haplotype of rs1799931*rs2087852, accounting for 27% of the total enrolled subjects and 3 cases developed hepatotoxicity, the incidence was 21.4% ( 3/14), which is significantly higher than 2.6% ( 1/38) of the low-risk group. 13 cases carry the high-risk haplotype of rs1799931*rs11996129, accounting for 26% of the total enrolled subjects and 3 cases developed hepatotoxicity, the incidence was 23.1% ( 3/13) which is also significantly higher than 2.7% ( 1/37). This result indicates the combination of rs1799931 and rs2087852 or rs1799931 and rs11996129 are associated with the incidence of hepatotoxicity. However, the statistical analysis of the 400+cases collected in the past failed to confirm such correlation, indicating the variation of the results may be higher due to lower number of enrolled subjects and lower incidence of hepatotoxicity. In the future, increased number of enrolled subjects will be helpful for validating the prediction capability of this combination. Nevertheless, based on the obtained results, patients who carry the high-risk haplotypes of rs1961456*rs1799931 have a higher risk of developing hepatotoxicity when receiving the first-line anti-TB drug treatment such as isoniazid and detection of this NAT2 haplotype during the treatment process will help the clinicians monitor the treatment progress of patients, reduce poor obedience or discontinued use of medications caused by side effects, and improve the control rate for TB.

### EXAMPLE 4

### Evaluation of the Effect of Hepatotoxical Side Effect in Patients Carry High-Risk Haplotype by Quantitative Indicators Serum Aminotransferases

Variation of the serum levels of aspartate aminotransferase (AST) and alanine aminotransferase (ALT) in the TB patients before and after drug administration were further assessed and we found a total of 4 kinds of SNPs,combination of high-risk groups, significant increase of the serum aminotransferase concentrations in these patients were observed and said increase is significantly different when compared with the low-risk group: rs1961456*rs1799931, rs1799931*rs2087852, rs1799931*rs1041983 and rs1799931*rs11996129. Among them, the mean of peak values of the changes in the serum ALT in the patients who carry the high-risk haplotype of rs1961456*rs1799931 was significantly higher than the low-risk group (43.7±23 vs. 29±18, p<0.05); as for the high-risk group that carry rs1799931*rs1041983 or rs1799931*rs11996129 the mean of peak values of the changes in the serum AST in the patients was significantly higher than the low-risk group. The means of peak values of the changes in the serum ALT and AST in the patients who carry the high-risk haplotype of rs1799931*rs2087852 were significantly higher than the low-risk group.

These statistical results and the changes in the serum ALT and AST levels of the patients who developed hepatotoxicity may due to skewed distribution, but previous studies have suggested that variations of the NAT2 haplotypes may be used as a substitution indicator for the activity of metabolic enzymes. Different enzymatic activities of the high-risk group may affect the development of hepatotoxicity through changing drug metabolism. In addition, ALT (alanine

Aminotransferase) is more specific when used for evaluation liver injury than AST. Therefore, TB patients who carry the high-risk haplotype of rs1961456*rs1799931 or rs1799931*rs2087852 showed a more significant increase in the ALT level when compared with the low-risk group and hepatotoxicity was more common in this group of patients.

### EXAMPLE 5

The analysis of the allele frequency of 5 SNPs of xanthine oxidase of 205 subjects were completed. Some genotype analyses of the samples were not obtained due to poor DNA quality, among which the allele frequency of rs1884725 and 2295475 and the distribution of wild-type/mutant allele was 66.3/33.6 and 41.5/58.5 percent, respectively; genotype distribution was 66.3% and 41.5% for wildtype, 30.2% and 44.4% for heterozygous mutant, and 3.4% and 14.1% for homozygous mutant; for rs17011368, the distribution of wild-type/mutant allele was 92.2/7.8 percent, genotype distribution was 92.2% for wildtype and 7.8% for heterozygous mutant. No homozygote genotype was found in the samples; in addition, for the analysis of allele frequency of rs566352 and rs72549369, only the wildtype genotypes were found in these samples and no mutant genotype (Table 4) was identified. For the test on different genotypes of Xanthine oxidase and PZA drug-induced hepatotoxicity, analyses of the gene samples from 119 TB patients and the results of 5 SNPs of XO were obtained. The 3 SNP variation, rs1884725, rs2295475 and rs17011368, the odds ratio of SNP to PZA drug-induced hepatotoxicity and SNP rs2295475 to PZA drug-induced hepatotoxicity (odds ratio) was 11.335 (p=0.000) and 14.883 (p=0.000), respectively (Table 5).

TABLE 5
Risk analysis of SNP haplotype combinations of
Xanthine Oxidase and TB drug-induced hepatic
injury

| | Hepatotoxicity | | | |
|---|---|---|---|---|
| SNP ID. | Present | Absent | Odds ratio | p value |
| rs1884725 | | | | |
| GG | 16 | 57 | 11.335 | 0.000 |
| GA + AA | 35 | 11 | | |
| rs2295475 | | | | |
| GG | 4 | 38 | 14.883 | 0.000 |
| GA + AA | 47 | 30 | | |
| rs17011368 | | | | |
| TT | 47 | 56 | 0.397 | 0.099 |
| TC + CC | 4 | 12 | | |

Note:
The heterozygous alleles shown in the table are not in order. For example, there is no difference between the GA and AG genotype of rs1799931 and both are presented in GA, same for other SNPs.

### EXAMPLE 6

The number of enrolled TB patients was increased to more than 400 cases and their genotype test results as well as clinical data were used for analysis. In the analysis of the correlation between SNP and TB drug-induced hepatotoxicity, we found the results are consistent with past analysis (see Table 6 for details).

TABLE 6
Risk analysis of SNP combinations of NAT2, CYP2E1 and
Xanthine Oxidase and TB drug-induced hepatic injury

| | | Hepatotoxicity | | | |
|---|---|---|---|---|---|
| | | Absent | Present | | |
| | Genotype | (% of each | (% of each | | |
| | (% of population) | group) | group) | Odds Ratio | p-value |
| NAT2 | | | | | |
| rs1799930 | GG 215 (52.7%) | 182 (84.7%) | 33 (15.3%) | — | |
| | GA 168 (41.2%) | 135 (80.4%) | 33 (19.6%) | 1.348 | 0.271 |
| | AA 25 (6.1%) | 15 (60.0%) | 10 (40.0%) | 3.677 | 0.004 |
| rs1041983 | CC 128 (30.8%) | 111 (86.7%) | 17 (13.3%) | — | |
| | CT 210 (50.5%) | 176 (83.8%) | 34 (16.2%) | 1.261 | 0.469 |
| | TT 78 (18.8%) | 52 (66.7%) | 26 (33.3%) | 3.265 | 0.001 |
| rs1112005 | CC 225 (55.1%) | 192 (85.3%) | 33 (14.7%) | — | |
| | CT 152 (37.3%) | 122 (80.3%) | 30 (19.7%) | 1.431 | 0.197 |
| | TT 31 (7.6%) | 20 (64.5%) | 11 (35.5%) | 3.200 | 0.006 |
| rs1495741 | GG 95 (23.2%) | 82 (86.3%) | 13 (13.7%) | — | |
| | AG 217 (53.1%) | 184 (84.8%) | 33 (15.2%) | 1.131 | 0.727 |
| | AA 97 (23.7%) | 66 (68.0%) | 31 (32.0%) | 2.963 | 0.003 |
| rs1199612 | TT 219 (53.0%) | 186 (84.9%) | 33 (15.1%) | — | |
| | TC 159 (38.5%) | 127 (79.9%) | 32 (20.1%) | 1.420 | 0.200 |
| | CC 35 (8.5%) | 23 (65.7%) | 12 (34.3%) | 2.941 | 0.007 |
| rs1961456 | GG 183 (44.0%) | 156 (85.2%) | 27 (14.8%) | — | |
| | GA 177 (42.5%) | 146 (82.5%) | 31 (17.5%) | 1.227 | 0.477 |
| | AA 56 (13.5%) | 38 (67.9%) | 18 (32.1%) | 2.737 | 0.004 |
| rs2087852 | TT 218 (52.3%) | 185 (84.9%) | 33 (15.1%) | — | |
| | TC 148 (35.5%) | 119 (80.4%) | 29 (19.6%) | 1.366 | 0.266 |
| | CC 51(12.2%) | 36 (70.6%) | 15 (29.4%) | 2.336 | 0.019 |
| rs1799929 | CC 366 (91.5%) | 299 (81.7%) | 67 (18.3%) | — | |
| | CT 33 (8.3%) | 25 (75.8%) | 8 (24.2%) | 1.428 | 0.405 |
| | TT 1 (0.3%) | 1 (100.0% | 0 (0.0%) | 0.000 | 1.000 |
| rs1799931 | GG 285 (69.2%) | 236 (82.8%) | 49 (17.2%) | — | |
| | GA 118 (28.6%) | 92 (78.0%) | 26 (22.0%) | 1.361 | 0.257 |
| | AA 9 (2.2%) | 7 (77.8%) | 2 (22.2%) | 1.376 | 0.696 |
| rs1801280 | TT 373 (91.0%) | 305 (81.8%) | 68 (18.2%) | — | |
| | TC 35 (8.5%) | 26 (74.3%) | 9 (25.7%) | 1.553 | 0.282 |
| | CC 2 (0.5%) | 2 (100.0% | 0 (0.0%) | 0.000 | 0.999 |
| rs1208 | AA 366 (90.8%) | 299 (81.7%) | 67 (18.3%) | — | |
| | AG 36 (8.9%) | 28 (77.8%) | 8 (22.2%) | 1.275 | 0.566 |
| | GG 1 (0.2%) | 1 (100.0% | 0 (0.0%) | 0.000 | 1.000 |

| Cytochrome P450 | | | | | |
|---|---|---|---|---|---|
| rs2249695 | CC 150(36.1%) | 116 (77.3%) | 34 (22.7%) | — | |
| | CT 205 (49.4%) | 175 (85.4%) | 30 (14.6%) | 0.585 | 0.053 |
| | TT 60(14.5%) | 47 (78.3%) | 13 (21.7%) | 0.944 | 0.875 |
| rs2031920 | CC 248 (62.5%) | 200 (80.6%) | 48 (19.4%) | — | |
| | CT 131 (33.0%) | 108 (82.4%) | 23 (17.6%) | 0.887 | 0.670 |
| | TT 18 (4.5%) | 14 (77.8%) | 4 (22.2%) | 1.190 | 0.767 |
| rs3813867 | GG 252 (62.1%) | 204 (81.0%) | 48 (19.0%) | — | |
| | GC 137 (33.7%) | 114 (83.2%) | 23 (16.8%) | 0.857 | 0.582 |
| | CC 17 (4.2%) | 13 (76.5%) | 4 (23.5%) | 1.308 | 0.651 |
| rs3813865 | GG 263 (63.2%) | 213 (81.0%) | 50 (19.0%) | — | |
| | GC 140(33.7%) | 117 (83.6%) | 23 (16.4%) | 0.837 | 0.522 |
| | CC 13 (3.1%) | 9 (69.2%) | 4 (30.8%) | 1.893 | 0.304 |
| Xanthine | | | | | |
| rs2295475 | GG 178 (43.4%) | 147 (82.6%) | 31 (17.4%) | — | |
| | GA 186 (45.4%) | 156 (83.9%) | 30 (16.1%) | 0.912 | 0.743 |
| | AA 46 (11.2%) | 30 (65.2%) | 16 (34.8%) | 2.529 | 0.012 |
| rs1884725 | GG 290 (71.1%) | 235 (81.0%) | 55 (19.0%) | — | |
| | GA 109 (26.7%) | 91 (83.5%) | 18 (16.5%) | 0.845 | 0.573 |
| | AA 9 (2.2%) | 6 (66.7%) | 3 (33.3%) | 2.136 | 0.294 |

Note:
The heterozygous alleles shown in the table are not in order. For example, there is no difference between the GA and AG genotype of rs1799931 and both are presented in GA, same for other SNPs.

### EXAMPLE 7

Based on the Examples 1-6 mentioned above, we further analyzed any two or three SNP genotype combinations of the three metabolic enzymes NAT2, CYP2E1 and Xanthine Oxidase to identify the best haplotype combination for predicting the risk of hepatotoxicity. See Table 7 and Table 8 for the results.

TABLE 7
Risk analysis of SNP genotype combinations of NAT2, CYP2E1 and
Xanthine Oxidase and TB drug-induced hepatic injury

| | Risk | | | | |
|---|---|---|---|---|---|
| | group | Hepatotoxicity | | | |
| | (% of | Absent | Present | Odds | |
| Grouping | population) | (% of each group) | (% of each group) | Ratio | p-value |
| rs2295475_rs1041983 | Low | 258 (86.9%) | 39 (13.1%) | 3.444 | 0.000 |
| | risk (72.8%) | | | | |
| | High | 73 (65.8%) | 38 (34.2%) | | |
| | risk (27.2%) | | | | |
| rs1495741_rs2295475 | Low | 244 (87.5%) | 35 (12.5%) | 3.366 | 0.000 |
| | risk (68.4%) | | | | |
| | High | 87 (67.4%) | 42 (32.6%) | | |
| | risk (31.6%) | | | | |
| rs1799930_rs1112005 | Low | 311 (83.2%) | 63 (16.8%) | 3.291 | 0.013 |
| | risk (93.7%) | | | | |
| | High | 15 (60.0%) | 10 (40.0%) | | |
| | risk (6.3%) | | | | |
| rs1961456_rs1799930 | Low | 317 (83.0%) | 65 (17.0%) | 3.251 | 0.013 |
| | risk (93.9%) | | | | |
| | High | 15 (60.0%) | 10 (40.0%) | | |
| | risk (6.1%) | | | | |
| rs2087852_rs1799930 | Low | 314 (82.6%) | 66 (17.4%) | 3.172 | 0.014 |
| | risk (93.8%) | | | | |
| | High | 15 (60.0%) | 10 (40.0%) | | |
| | risk (6.2%) | | | | |
| rs11996129_rs1799930 | Low | 312 (82.5%) | 66 (17.5%) | 3.152 | 0.014 |
| | risk (93.8%) | | | | |
| | High | 15 (60.0%) | 10 (40.0%) | | |
| | risk (6.2%) | | | | |
| rs1495741_rs11996129 | Low | 278 (84.8%) | 50 (15.2%) | 3.064 | 0.000 |
| | risk (81.2%) | | | | |
| | High | 49 (64.5%) | 27 (35.5%) | | |
| | risk (18.8%) | | | | |
| rs2295475_rs1961456 | Low | 269 (85.7%) | 45 (14.3%) | 3.038 | 0.000 |
| | risk (77.3%) | | | | |
| | High | 61 (66.3%) | 31 (33.7%) | | |
| | risk (22.7%) | | | | |
| rs1495741_rs1961456 | Low | 276 (85.2%) | 48 (14.8%) | 2.981 | 0.000 |
| | risk (79.8%) | | | | |
| | High | 54 (65.9%) | 28 (34.1%) | | |
| | risk (20.2%) | | | | |
| rs1799931_rs1799930 | Low | 290 (84.3%) | 54 (15.7%) | 2.954 | 0.001 |
| | risk (84.7%) | | | | |
| | High | 40 (64.5%) | 22 (35.5%) | | |
| | risk (15.3%) | | | | |
| rs1495741_rs1799930 | Low | 275 (84.6%) | 50 (15.4%) | 2.918 | 0.000 |
| | risk (81.3%) | | | | |
| | High | 49 (65.3%) | 26 (34.7%) | | |
| | risk (18.8%) | | | | |
| rs1961456_rs1112005 | Low | 299 (84.2%) | 56 (15.8%) | 2.912 | 0.002 |
| | risk (87.4%) | | | | |
| | High | 33 (64.7%) | 18 (35.3%) | | |
| | risk (12.6%) | | | | |
| rs1495741_rs1112005 | Low | 279 (84.8%) | 50 (15.2%) | 2.911 | 0.001 |
| | risk (82.5%) | | | | |
| | High | 46 (65.7%) | 24 (34.3%) | | |
| | risk (17.5%) | | | | |
| rs11996129_rs1041983 | Low | 289 (84.5%) | 53 (15.5%) | 2.908 | 0.001 |
| | risk (83.2%) | | | | |
| | High | 45 (65.2%) | 24 (34.8%) | | |
| | risk (16.8%) | | | | |
| rs1799931_rs1041983 | Low | 289 (84.5%) | 53 (15.5%) | 2.908 | 0.001 |
| | risk (83.2%) | | | | |
| | High | 45 (65.2%) | 24 (34.8%) | | |
| | risk (16.8%) | | | | |
| rs1961456_rs1041983 | Low | 278 (85.5%) | 47 (14.5%) | 2.907 | 0.000 |
| | risk (78.7%) | | | | |
| | High | 59 (67.0%) | 29 (33.0%) | | |
| | risk (21.3%) | | | | |
| rs1961456_rs1799931 | Low | 267 (85.9%) | 44 (14.1%) | 2.856 | 0.000 |
| | risk (75.7%) | | | | |
| | High | 68 (68.0%) | 32 (32.0%) | | |
| | risk (24.3%) | | | | |
| rs1495741_rs1041983 | Low | 265 (85.5%) | 45 (14.5%) | 2.855 | 0.000 |
| | risk (76.0%) | | | | |
| | High | 66 (67.3%) | 32 (32.7%) | | |
| | risk (24.0%) | | | | |
| rs1495741_rs2087852 | Low | 279 (84.5%) | 51 (15.5%) | 2.845 | 0.001 |
| | risk (81.3%) | | | | |
| | High | 50 (65.8%) | 26 (34.2%) | | |
| | risk (18.7%) | | | | |
| rs1799930_rs1041983 | Low | 287 (84.4%) | 53 (15.6%) | 2.831 | 0.001 |
| | risk (83.5%) | | | | |
| | High | 44 (65.7%) | 23 (34.3%) | | |
| | risk (16.5%) | | | | |
| rs2249695_rs1112005 | Low | 281 (85.2%) | 49 (14.8%) | 2.811 | 0.000 |
| | risk (81.3%) | | | | |
| | High | 51 (67.1%) | 25 (32.9%) | | |
| | risk (18.7%) | | | | |
| rs11996129_rs1961456 | Low | 313 (83.0%) | 64 (17.0%) | 2.795 | 0.010 |
| | risk (92.0%) | | | | |
| | High | 21 (63.6%) | 12 (36.4%) | | |
| | risk (8.0%) | | | | |
| rs2087852_rs1041983 | Low | 291 (84.3%) | 54 (15.7%) | 2.754 | 0.001 |
| | risk (83.5%) | | | | |
| | High | 45 (66.2%) | 23 (33.8%) | | |
| | risk (16.5%) | | | | |
| rs11996129_rs2087852 | Low | 313 (82.8%) | 65 (17.2%) | 2.752 | 0.017 |
| | risk (92.0%) | | | | |
| | High | 21 (63.6%) | 12 (36.4%) | | |
| | risk (8.0%) | | | | |
| rs1495741_rs1799931 | Low | 268 (84.8%) | 48 (15.2%) | 2.744 | 0.000 |
| | risk (78.2%) | | | | |
| | High | 59 (67.0%) | 29 (33.0%) | | |
| | risk (21.8%) | | | | |
| rs1112005_rs1041983 | Low | 290 (84.5%) | 53 (15.5%) | 2.736 | 0.002 |
| | risk (84.5%) | | | | |
| | High | 42 (66.7%) | 21 (33.3%) | | |
| | risk (15.5%) | | | | |
| rs11996129_rs1799931 | Low | 283 (84.2%) | 53 (15.8%) | 2.727 | 0.001 |
| | risk (82.6%) | | | | |
| | High | 47 (66.2%) | 24 (33.8%) | | |
| | risk (17.4%) | | | | |
| rs2087852_rs1112005 | Low | 312 (83.2%) | 63 (16.8%) | 2.724 | 0.015 |
| | risk (92.4%) | | | | |
| | High | 20 (64.5%) | 11 (35.5%) | | |
| | risk (7.6%) | | | | |
| rs2249695_rs1799930 | Low | 275 (84.6%) | 50 (15.4%) | 2.698 | 0.001 |
| | risk (80.4%) | | | | |
| | High | 53 (67.1%) | 26 (32.9%) | | |
| | risk (19.6%) | | | | |
| rs11996129_rs1112005 | Low | 309 (83.1%) | 63 (16.9%) | 2.698 | 0.016 |
| | risk (92.3%) | | | | |
| | High | 20 (64.5%) | 11 (35.5%) | | |
| | risk (7.7%) | | | | |
| rs2087852_rs1961456 | Low | 301 (83.8%) | 58 (16.2%) | 2.669 | 0.004 |
| | risk (87.1%) | | | | |
| | High | 35 (66.0%) | 18 (34.0%) | | |
| | risk (12.9%) | | | | |
| rs1799931_rs1112005 | Low | 286 (84.1%) | 54 (15.9%) | 2.463 | 0.004 |
| | risk (84.4%) | | | | |
| | High | 43 (68.3%) | 20 (31.7%) | | |
| | risk (15.6%) | | | | |
| rs2087852_rs1799931 | Low | 270 (84.1%) | 51 (15.9%) | 2.220 | 0.005 |
| | risk (78.5%) | | | | |
| | High | 62 (70.5%) | 26 (29.5%) | | |
| | risk (21.5%) | | | | |
| rs2295475_rs2087852 | Low | 129 (83.2%) | 26 (16.8%) | 1.259 | 0.435 |
| | risk (38.1%) | | | | |
| | High | 201 (79.8%) | 51 (20.2%) | | |
| | risk (61.9%) | | | | |
| rs2295475_rs11996129 | Low | 134 (82.7%) | 28 (17.3%) | 1.215 | 0.519 |
| | risk (40.1%) | | | | |
| | High | 193 (79.8%) | 49 (20.2%) | | |
| | risk (59.9%) | | | | |
| rs2295475_rs1799930 | Low | 136 (82.9%) | 28 (17.1%) | 1.240 | 0.439 |
| | risk (41.0%) | | | | |
| | High | 188 (79.7%) | 48 (20.3%) | | |
| | risk (59.0%) | | | | |

TABLE 8
Risk analysis of the genotype combinations of NAT2, CYP2E1
and Xanthine Oxidase and TB drug-induced hepatic injury

| | Risk | | Hepatotoxicity | | | |
|---|---|---|---|---|---|---|
| | group | | Absent | Present | | |
| | (% of | | (% of each | (% of each | | |
| | population) | | group) | group) | Odds Ratio | p-value |
| rs1495741 vs. | Low risk | 274 (67.3%) | 240 (87.6%) | 34 (12.4%) | 3.373 | 0.000 |
| rs2295475 vs. | High risk | 133 (32.7%) | 90 (67.7%) | 43 (32.3%) | | |
| rs1041983 | | | | | | |
| rs1112005 vs. | Low risk | 271 (68.1%) | 236 (87.1%) | 35 (12.9%) | 2.988 | 0.000 |
| rs2295475 vs. | High risk | 127 (31.9%) | 88 (69.3%) | 39 (30.7%) | | |
| rs1799929 | | | | | | |
| rs1112005 vs. | Low risk | 270 (67.7%) | 235 (87.0%) | 35 (13.0%) | 3.128 | 0.000 |
| rs2295475 vs. | High risk | 129 (32.3%) | 88 (68.2%) | 41 (31.8%) | | |
| rs1799930 | | | | | | |
| rs1112005 vs. | Low risk | 274 (68.0%) | 239 (87.2%) | 35 (12.8%) | 3.297 | 0.000 |
| rs2295475 vs. | High risk | 129 (32.0%) | 87 (67.4%) | 42 (32.6%) | | |
| rs1799931 | | | | | | |
| rs1112005 vs. | Low risk | 267 (65.9%) | 234 (87.6%) | 33 (12.4%) | 3.210 | 0.000 |
| rs2295475 vs. | High risk | 138 (34.1%) | 95 (68.8%) | 43 (31.2%) | | |
| rs1961456 | | | | | | |
| rs1112005 vs. | Low risk | 261 (64.4%) | 229 (87.7%) | 32 (12.3%) | 3.253 | 0.000 |
| rs2295475 vs. | High risk | 144 (35.6%) | 99 (68.8%) | 45 (31.3%) | | |
| rs2087852 | | | | | | |
| rs1112005 vs. | Low risk | 183 (45.3%) | 161 (88.0%) | 22 (12.0%) | 2.425 | 0.001 |
| rs2295475 vs. | High risk | 221 (54.7%) | 166 (75.1%) | 55 (24.9%) | | |
| rs2249695 | | | | | | |
| rs1112005 vs. | Low risk | 273 (67.7%) | 238 (87.2%) | 35 (12.8%) | 3.245 | 0.000 |
| rs2295475 vs. | High risk | 130 (32.3%) | 88 (67.7%) | 42 (32.3%) | | |
| rs11996129 | | | | | | |
| rs1961456 vs. | Low risk | 301 (73.4%) | 259 (86.0%) | 42 (14.0%) | 2.796 | 0.000 |
| rs1799931 vs. | High risk | 109 (26.6%) | 75 (68.8%) | 34 (31.2%) | | |
| rs1041983 | | | | | | |
| rs1961456 vs. | Low risk | 272 (67.5%) | 239 (87.9%) | 33 (12.1%) | 3.539 | 0.000 |
| rs1799931 vs. | High risk | 131 (32.5%) | 88 (67.2%) | 43 (32.8%) | | |
| rs2295475 | | | | | | |

### EXAMPLE 8

Twenty-one healthy subjects were enrolled and a single dose of a known substrate of NAT2, isoniazid (INH), was given orally and blood samples were collected at 0.25, 0.5, 0.75, 1, 1.5, 2, 3, 4, 6, 7, 8, 12 and 24 hours after administration to measure the concentration of the metabolite of INH, acetyl isoniazid (AINH), in the blood after NAT2 metabolism and the ratio of blood AINH level to INH level of each subject was used to calculate the in vivo metabolic activity of NAT2 for representation of the possible pharmacological effect(s) of the drug. The results (Table 9) suggest that the high- and low-risk grouping of the multiple genotype combinations is correlated to the pharmacological effect(s) of NAT2. This finding demonstrates the haplotype combinations disclosed in this invention, which not only can be used for predicting anti-TB drug-induced hepatotoxicity but also can be applied for reasonable prediction of the relationship between other drugs that are related to the pharmacological effect(s) of the haplotype combination of the enzymes and diseases. For example, drugs that are metabolized by NAT2: sulfamethazine, sulphonamides, hydralazine, aminoglutethimide, aminosalicylate sodium, p-anisidine, 2-aminofluorene, sulfadiazine, sulfasalazine, procainamide, dapsone, nitrazepam, hydralazine, zonisamide and isoniazid; drugs that are metabolized by Xanthine oxidase: azathioprine, mercaptopurine, theophylline, pyrazinamide and so on; drugs that are metabolized by CYP2E1: Halothane, Enflurane, Isoflurane, paracetamol, dapsone, theophylline, ethanol, chlorzoxazone, toluene, Isoniazid and so on.

TABLE 9
Correlation between the combinations of NAT2 SNP genotypes and
the pharmacological effects of NAT2

| | Risk | | NAT2 activity* | | |
|---|---|---|---|---|---|
| Grouping | group | N | Mean | SD | Sig. |
| rs1495741_rs1041983 | Low risk | 13 | 1.69 | 0.56 | |
| | High risk | 8 | 0.70 | 0.57 | 0.001 |
| rs1495741_rs1112005 | Low risk | 16 | 1.57 | 0.65 | |
| | High risk | 5 | 0.50 | 0.16 | 0.002 |
| rs1495741_rs1799931 | Low risk | 16 | 1.58 | 0.64 | |
| | High risk | 5 | 0.47 | 0.14 | 0.001 |
| rs1495741_rs1961456 | Low risk | 16 | 1.57 | 0.65 | |
| | High risk | 5 | 0.50 | 0.16 | 0.002 |
| rs1495741_rs2087852 | Low risk | 16 | 1.57 | 0.65 | |
| | High risk | 5 | 0.50 | 0.16 | 0.002 |
| rs1495741_rs11996129 | Low risk | 16 | 1.57 | 0.65 | |
| | High risk | 5 | 0.50 | 0.16 | 0.002 |
| rs11996129_rs1041983 | Low risk | 17 | 1.51 | 0.68 | |
| | High risk | 4 | 0.48 | 0.18 | 0.009 |
| rs11996129_rs1799931 | Low risk | 16 | 1.56 | 0.66 | |
| | High risk | 5 | 0.51 | 0.17 | 0.003 |
| rs2087852_rs1041983 | Low risk | 17 | 1.51 | 0.68 | |
| | High risk | 4 | 0.48 | 0.18 | 0.009 |
| rs2087852_rs1799931 | Low risk | 15 | 1.55 | 0.68 | |
| | High risk | 6 | 0.73 | 0.54 | 0.017 |
| rs1961456_rs1041983 | Low risk | 16 | 1.52 | 0.70 | |
| | High risk | 5 | 0.63 | 0.37 | 0.014 |

*The NAT2 activity was presented as the metabolic ratio calculated as AUCt ratios for AINH to INH

### EXAMPLE 9

In addition to the correlation with hepatotoxicity, 124 subjects were enrolled and the concentration of uric acid in their urine samples were analyzed. We further found the correlation between XO SNP and metabolism of uric acid in vivo. Subjects who have XO SNP rs1884725 as GA or AA genotype and/or rs2295475 as GA or AA genotype showed higher in vivo uric acid concentration. This result can be further applied in prediction of the risk of the diseases relating to metabolism of uric acid, such as gout and prediction of the incidence of high uric acid and its related diseases after administration of the drugs that are known to affect the concentration of uric acid.

TABLE 10
Correlation between XO SNP genotypes and the
concentration of uric acid in the body

| SNP ID | Uric acid (mg/dL) | sig.* |
|---|---|---|
| rs1884725 | | |
| GG | 7.0 ± 3.1 | |
| GA | 9.5 ± 3.0 | <0.001 |
| AA | 11.4 ± 2.6 | <0.001 |
| rs2295475 | | |
| GG | 6.0 ± 2.5 | |
| GA | 8.1 ± 2.8 | <0.001 |
| AA | 11.8 ± 2.7 | <0.001 |

*Compare to the wild type group (rs1884725: GG; rs2295475: GG).
Note:
The representations of the heterozygous alleles are not in order, for example, the GA and AG genotypes of rs1799931 are both represented in AG, same for other SNPs.

The SNP genotype of the target gene used in the method for screening the risk of drug-induced toxicity disclosed in the aforementioned examples can be tested by known industry methods; the methods for analysis, detection, measurement, identification and/or confirmation of the SNP genotype of the target gene are well-known in the industry, including but not limited to, at least one of the following methods: restriction fragment length polymorphism (RFLP), tetra-primer ARMS-PCR, PCR molecular beacons, SNP microarrays, temperature gradient gel electrophoresis and denaturing high performance liquid chromatography.

The foregoing detailed description of the invention and the specific examples are provided herein for the purpose of illustration only, and the invention is not limited to the preferred embodiments shown. It should be understood that any changes or modifications within the spirit of the invention shall be included in the scope of present invention.

What is claimed is:

1. A method for screening the risk of drug-induced toxicity, said method is consisting of the following steps:
Step 1: obtain the test specimen from test subjects;
Step 2: examine the single nucleotide polymorphism (SNP) of the genomic DNA of at least one target gene of the test specimen, wherein the target gene is consisting of NAT2, CYP2E1 and Xanthine Oxidase;
Step 3: based on the genotype of the SNP of the target gene, determine whether the sample is of high-risk group of drug-induced toxicity or can be used as a drug therapy or prognostic indicator.

2. The method as recited in claim 1, wherein the drug is one of or the combination of an anti-TB drug and a drug for treating other disease.

3. The method as recited in claim 2, wherein the anti-TB drug is one of or the combination of isoniazid, rifampin (RMP), pyrazinamide (PZA) and ethambutol.

4. The method as recited in claim 1, wherein the toxicity is hepatotoxicity and/or increased ALT or AST level.

5. The method as recited in claim 1, wherein the test specimen are mammalian samples.

6. The method as recited in claim 1, wherein the test specimen is blood, amniotic fluid, cerebrospinal fluid, organ, tissue, cell, lymphatic fluid, tissue fluid, other body fluids, skin, hair, muscle, placenta, gastrointestinal tract and oral mucosa.

7. The method as recited in claim 1, wherein the disclosed SNP is at least one of the following: rs1041983, rs1112005, rs1495741, rs1799930, rs1799931, rs1801280, rs1961456, rs2087852, rs11996129, rs2031920, rs2249695, rs3813865, rs3813867, rs1884725, rs2295475 and rs17011368.

8. The method as recited in claim 7, wherein when the genotype of the SNP rs1041983 is TT, TC or CT; the genotype of SNP rs1112005 is TT, TC or CT; the genotype of SNP rs1495741 is AA; the genotype of SNP rs1799930 is AA, AG or GA; the genotype of SNP rs1799931is AA, AG or GA; the genotype of SNP rs1801280 is CC, CT or TC; the genotype of SNP rs1961456 is AA, AG or GA; the genotype of SNP rs2087852 is CC, CT or TC; the genotype of SNP rs11996129 is CC, CT or TC; the genotype of SNP rs2031920 is CC; the genotype of SNP rs2249695 is CC; the genotype of SNP rs3813865 is GG; the genotype of SNP rs3813867 is GG; the genotype of SNP rs1884725 is AA, AG or GA; the genotype of SNP rs2295475 is AA, AG or GA; or when the genotype of SNP rs17011368 is TT, said individual has increased risk for developing drug-induced toxicity.

9. The method as recited in claim 8, wherein when the genotype of the disclosed SNP rs1495741 is AA or the genotype of SNP rs2295475is AA, said individual has a higher risk of induced toxicity.

10. The method as recited in claim 1, wherein the target gene NAT2 is the nucleotide sequence indicated in GenBank Accession Number: NC_000008.10; the target gene CYP2E1 is the nucleotide sequence indicated in GenBank Accession Number: NC_000010.10; the target gene Xanthine Oxidase is the nucleotide sequence indicated in GenBank Accession Number: NC_000002.11.

11. A method for screening high uric acid and its related diseases, such method is consisting of the following steps:
Step 1: obtain the test specimen from test subjects;
Step 2: examine the single nucleotide polymorphism (SNP) of the genomic DNA of Xanthine Oxidase of the test specimen;
Step 3: based on the genotype of the SNP of the Xanthine Oxidase gene, determine whether the sample is of the high-risk group of high uric acid and its related diseases or whether it can be used as a drug therapy or prognostic indicator.

12. The method as recited in claim 11, wherein the test specimen are mammalian samples.

13. The method as recited in claim 11, wherein the test specimen is blood, amniotic fluid, cerebrospinal fluid, organ, tissue, cell, lymphatic fluid, tissue fluid, other body fluids, skin, hair, muscle, placenta, gastrointestinal tract and oral mucosa.

14. The method as recited in claim 11, wherein the SNP is at least one of the following: rs1884725 and rs2295475.

15. The method as recited in claim 14, wherein when the genotype of SNP rs1884725 is GA, AG or AA or when the genotype of SNP rs2295475 is GA, AG or AA, said individual has increased risk of developing high uric acid and its related diseases.

16. The method as recited in claim 11, wherein the Xanthine Oxidase gene is the nucleotide sequence as indicated in the GenBank Accession Number: NC_000002.11.
